(19) United States
(12) Patent Application Publication
(10) Pub. No.: US 2024/0361265 A1
(43) Pub. Date: Oct. 31, 2024

(54) APPARATUS AND METHODS FOR EXTRACELLULAR IMPEDANCE SPECTROSCOPY OF BARRIER CELLS

(71) Applicants: Georgia Tech Research Corporation, Atlanta, GA (US); National Institutes of Health, Bethesda, MD (US)

(72) Inventors: Craig Richard Forest, Atlanta, GA (US); Kapil Bharati, Potomac, MD (US); Colby Lewallan, North Bethesda, MD (US); Arvydas Maminishkis, Washington, DC (US)

(21) Appl. No.: 18/412,842

(22) Filed: Jan. 15, 2024

Related U.S. Application Data
(60) Provisional application No. 63/479,798, filed on Jan. 13, 2023.

(51) Int. Cl. G01N 27/02; G01N 33/50

(52) U.S. Cl. CPC G01N 27/026; G01N 33/5008

(57) ABSTRACT

An exemplary cellular analysis system and method are disclosed that can determine epithelial cell membrane properties by fitting electrochemical impedance spectroscopy (EIS) measurements to (i) an intracellular circuit model having electrical property parameters of the epithelial cell membrane properties, including the apical, basolateral, and shunt pathways or (ii) an extracellular circuit model. The exemplary cellular analysis system and method can be used to measure membrane properties of epithelial electrophysiology that can beneficially be used to assess for transport dysfunction and/or evaluate cellular drug responses in disease models. An exemplary cellular analysis system and method are disclosed that can determine the polarization of epithelial cells in response to stimuli as a time-constant impedance-associated ratio (tau ratio) that can be used in a massively parallel screening system for drug discovery.

### CROSS REFERENCE TO RELATED APPLICATIONS

This application claims the benefit of priority to U.S. Provisional Application No. 63/479,798, filed Jan. 13, 2023, which is incorporated by reference herein in its entirety.

### GOVERNMENT SUPPORT CLAUSE

This invention was made with government support under Grant Nos. RO1NS102727, RF1AG079269, U01MH106027, and R01DA029639, awarded by the National Institutes of Health. The Government has certain rights in the invention.

### BACKGROUND

Epithelia are barrier-type cells that regulate the transport of materials into and out of the body. Dysfunction of these cells is implicated in numerous diseases, such as cystic fibrosis, age-related macular degeneration, and diabetes. Since the discovery of induced pluripotent stem cells (iPSCs), utilizing iPSC-based therapies has been used to halt and potentially reverse the progression of these diseases.

Assessment of extracellular electrophysiology, such as validation of tissue polarity and function, is beneficial for the assessment of new epithelia-based therapies. Common assessment approaches entail a thorough physiological exam and are commonly performed electrochemically. A common measurement of epithelial electrophysiology is trans-epithelial resistance (TER), which is used to assess the formation of tight junctions. Such methods either result in the destruction of the cells, are incomplete, difficult to perform, or provide low throughput.

There is a benefit to improving the assessment of extracellular electrophysiology.

### SUMMARY

An exemplary cellular analysis system and method are disclosed that can determine epithelial cell membrane properties by fitting electrochemical impedance spectroscopy (EIS) measurements to (i) an intracellular circuit model having electrical property parameters of the epithelial cell membrane properties, including the apical, basolateral, and shunt pathways or (ii) an extracellular circuit model. The exemplary cellular analysis system and method can be used to measure membrane properties of epithelial electrophysiology that can beneficially be used to assess for transport dysfunction and/or evaluate cellular drug responses in disease models. In some embodiments, the exemplary system and method are employed in a massively parallel screening system for drug discovery.

In an embodiment, the exemplary system and method employ a three-electrode measurement of the epithelial cell membrane that acquires two voltage divide measurements, one between two external electrodes placed in the bath of cell culture and a second between one of the external electrodes and an internal electrode placed within the cell membrane. In another embodiment, the exemplary system and method employ only external electrode measurements of the epithelial cell membrane. This exemplary system and method can characterize, in a non-destructive manner, membrane-specific evoked responses by enabling frequency modulation of the voltage signal on the epithelial tissue combined with fluid exchange on both the tissue's apical and basil sides to determine a time-constant impedance-associated ratio. The time-constant impedance-associated ratio can be employed to provide a platform for the standardized assessment of the potency of stem cell therapies.

A study was conducted that developed and validated the integrated extracellular and intracellular circuit models of epithelia with known resistances and capacitances, confirming a median error of 19%. The study used induced pluripotent stem cell (iPSC)-derived retinal pigment epithelial (RPE) tissue to quantify the well-known response of this tissue to the apical application of adenosine triphosphate (ATP).

In another embodiment, the exemplary system is configured to determine non-invasively the polarization of epithelial cells in response to stimuli as a time-constant impedance-associated ratio (tau ratio) by (i) fitting extracellular measurements acquired from an impedance spectroscopy instrument to an extracellular circuit and circuit model and (ii) using the elements in the circuit model in a time-constant impedance-associated ratio model. Notably, the time-constant impedance-associated ratio (tau ratio) is a parameter that can be determined from two electrodes positioned for extracellular measurements.

In an aspect, a system is disclosed (e.g., analysis system or measurement system having an analysis system) comprising: an analysis system (e.g., edge device, software, or cloud infrastructure) configured, via computer readable instructions to: receive at least two response signals, or values thereof, of a measurement of a cell sample acquired from an impedance spectroscopy device or an associated data storage, wherein the cell sample had a first cell side associated with an apical membrane and a second cell side associated with a basolateral membrane, wherein the at least two response signals or values thereof include (i) a first measurement across the first cell side or the second cell side between a first electrode and a second electrode and (ii) a second measurement across both the first and the second cell side between the first electrode and a third electrode; and determine, via an impedance model of intracellular electrophysiology of a cell, based on the received two response signals or values thereof, electrical characteristic values for the cell sample, wherein the impedance model includes at least a resistance and capacitance characteristics of the first cell side, a resistance and capacitance characteristics of the second cell side, resistance characteristics of solutions in contact with the first and second cell side (e.g., unique characteristics different between the two cell sides), and a resistance characteristic across the first and second cell side, wherein one or more of the electrical characteristic values are made accessible (e.g., via a graphical user interface or report) for use in a non-invasive characterization of the cell sample in response to a stimulus (e.g., therapeutics or environmental stimulus) applied to the cell sample or non-invasive characterization of the stimulus.

In some embodiments, the electrical characteristic values for the cell sample are determined by solving, via a mathematical solver, using the two response signals or values thereof at a plurality of frequencies acquired via the impedance spectroscopy device, a resistance value and a capacitance value of the first cell side, a resistance value and a capacitance value of the second cell side, a first resistance value and a second resistance value respective solutions in contact with the first and second cell side, and a resistance value across both the first and second cell side, wherein the at least two response signals or values thereof include (i) a first voltage measure across the first or the second cell side between the first electrode and the second electrode and (ii) a second voltage measure across both the first and the second cell side between the first electrode and the third electrode.

In some embodiments, the cell sample comprises a cell tissue.

In some embodiments, the first cell side is in contact with a first nutrient solution bath (e.g., salt bath), and wherein the second cell side is in contact with a second nutrient solution bath (e.g., salt bath), and wherein the first salt solution and the second salt solution are the same.

In some embodiments, the impedance model of extracellular electrophysiology of the cell assumes the first salt solution and the second salt solution have the same resistance value.

In some embodiments, the impedance model of extracellular electrophysiology of the cell assumes the first salt solution and the second salt solution have different resistance values.

In some embodiments, the first cell side is in contact with a first nutrient solution bath, and wherein the second cell side is in contact with a second nutrient solution bath, wherein the first salt solution and the second salt solution are different.

In some embodiments, the first electrode is operably coupled to the first salt solution bath, and wherein the second electrode is operably coupled to an intracellular cellular space defined between the first cell side and the second cell side.

In some embodiments, the impedance spectroscopy device comprises an impedance spectroscopy instrument.

In some embodiments, the impedance spectroscopy device is implemented as an external instrument driver device configured to couple to an impedance spectroscopy instrument.

In some embodiments, trends for the electrical characteristic values are made accessible for use in the characterization of the cell sample or a stimulus (e.g., therapeutics or environmental stimulus) applied to the cell sample.

In another aspect, a system is disclosed (e.g., analysis system or measurement system having an analysis system using tau ratio) comprising: an analysis system (e.g., edge device, software, or cloud infrastructure) configured, via computer readable instructions to: receive a response signal, or values thereof, of a measurement of a cell sample acquired from an impedance spectroscopy device or an associated data storage, wherein the cell sample had a first cell side associated with an apical membrane and a second cell side associated with a basolateral membrane, wherein the response signal or values thereof include (i) a first measurement across the first cell side or the second cell side between a first electrode and a second electrode; and determine, via an impedance model of extracellular electrophysiology of a cell, based on the received response signal or values thereof, a first electrical characteristic value for the cell sample, a second electrical characteristic value, and a time-constant impedance-associated ratio (tau ratio), wherein the first electrical characteristic value, the second electrical characteristic value, and the time-constant impedance-associated ratio are made accessible (e.g., via a graphical user interface or report) for use in a non-invasive characterization of the cell sample in response to a stimulus (e.g., therapeutics or environmental stimulus) applied to the cell sample.

In some embodiments, the electrical characteristic values for the cell sample are determined by solving, via a mathematical solver, using the response signal or values thereof at a plurality of frequencies acquired via the impedance spectroscopy device, a resistance value, and a capacitance value associated with the first cell side, a resistance value and a capacitance value associated with the second cell side, and a first resistance value and a second resistance value respective solutions in contact with the first and second cell side, wherein the response signal or values thereof include a voltage measure across the first electrode and the second electrode.

In some embodiments, the time-constant impedance-associated ratio can be used to provide an indication of membrane responses to the stimulus applied to the cell sample between the first cell side and the second cell side.

In some embodiments, the cell sample comprises a cell tissue.

In some embodiments, the first cell side is in contact with a first nutrient solution bath (e.g., salt bath), and wherein the second cell side is in contact with a second nutrient solution bath (e.g., salt bath), and wherein the first salt solution and the second salt solution are the same.

In some embodiments, the first cell side is in contact with a first nutrient solution bath, and wherein the second cell side is in contact with a second nutrient solution bath, wherein the first salt solution and the second salt solution are different.

In some embodiments, the impedance spectroscopy device comprises an impedance spectroscopy instrument.

In some embodiments, the impedance spectroscopy device is implemented as an external instrument driver device configured to couple to an impedance spectroscopy instrument.

In another aspect, a kit is disclosed comprising: a measurement chamber configured to house or culture a cell sample, wherein the cell sample once placed in the measurement chamber has a first cell side associated with an apical membrane and a second cell side associated with a basolateral membrane; and two or more electrodes configured to be place in the measurement chamber to measure a cell sample to be cultured or placed in the measurement chamber (e.g., epithelial tissues), the two or more electrodes having connectivity to an impedance spectroscopy device configured to interrogate the two or more electrodes once positioned in extracellular spaces, and optionally, intracellular spaces, to provide at least one response signals of the cell sample to current stimulation at different frequencies, wherein the at least one response signals of the cell sample can be employed in a model fitting operation to solve for electrical characteristic values for the cell sample, wherein one or more of the electrical characteristic values are made accessible (e.g., via a graphical user interface or report) for use in a non-invasive characterization of the cell sample in response to a stimulus (e.g., therapeutics or environmental stimulus) applied to the cell sample.

In some embodiments, the two or more electrodes consist only of electrodes positioned in extracellular spaces of the measurement chamber, and wherein the response signal provides for a first electrical characteristic value for the cell sample, a second electrical characteristic value, and a time-constant impedance-associated ratio (tau ratio) to be use in the non-invasive characterization of the cell sample.

In some embodiments, the two or more electrodes consist of only two electrodes positioned in extracellular spaces of the measurement chamber and a third electrode positioned in the intracellular space of the cell sample, and wherein the response signals include (i) a first voltage measurement across the first cell side or the second cell side between a first electrode and a second electrode and (ii) a second voltage measurement across both the first and the second cell side between the first electrode and a third electrode, and wherein the at least one response signals provides for a resistance and capacitance characteristics of the first cell side, a resistance and capacitance characteristics of the second cell side, resistance characteristics of solutions in contact with the first and second cell side, and a resistance characteristic across the first and second cell side.

In another aspect, a method is disclosed of characterizing cell sample or stimulus applied to the cell sample, the method comprising: receiving at least two response signals, or values thereof, of a measurement of a cell sample acquired from an impedance spectroscopy device or an associated data storage, wherein the cell sample had a first cell side associated with an apical membrane and a second cell side associated with a basolateral membrane, wherein the at least two response signals or values thereof include (i) a first measurement across the first cell side or the second cell side between a first electrode and a second electrode and (ii) a second measurement across both the first and the second cell side between the first electrode and a third electrode; and determining, via an impedance model of extracellular electrophysiology of a cell, based on the received two response signals or values thereof, electrical characteristic values for the cell sample, wherein the impedance model includes at least a resistance and capacitance characteristics of the first cell side, a resistance and capacitance characteristics of the second cell side, resistance characteristics of solutions in contact with the first and second cell side, and a resistance characteristic across the first and second cell side, wherein one or more of the electrical characteristic values are made accessible (e.g., via a graphical user interface or report) for use in a non-invasive characterization of the cell sample in response to a stimulus (e.g., therapeutics or environmental stimulus) applied to the cell sample or non-invasive characterization of the stimulus, wherein the stimulus comprises at least one of a therapeutic agent, disease agent, or compound.

In another aspect, a method is disclosed of characterizing cell sample or stimulus applied to the cell sample, the method comprising: receiving a response signal, or values thereof, of a measurement of a cell sample acquired from an impedance spectroscopy device or an associated data storage, wherein the cell sample had a first cell side associated with an apical membrane and a second cell side associated with a basolateral membrane, wherein the response signal or values thereof include (i) a first measurement across the first cell side or the second cell side between a first electrode and a second electrode; and determining, via an impedance model of extracellular electrophysiology of a cell, based on the received response signal or values thereof, a first electrical characteristic value for the cell sample, a second electrical characteristic value, and a time-constant impedance-associated ratio (tau ratio), wherein the first electrical characteristic value, the second electrical characteristic value, and the time-constant impedance-associated ratio are made accessible (e.g., via a graphical user interface or report) for use in a non-invasive characterization of the cell sample in response to a stimulus (e.g., therapeutics or environmental stimulus) applied to the cell sample, wherein the stimulus comprises at least one of a therapeutic agent, disease agent, or compound.

In some embodiments, the non-invasive characterization is employed to direct the application of the stimulus (e.g., introduce stimulus).

In some embodiments, the impedance spectroscopy device is configured to generate an interrogation signal comprising a varying sweep of frequencies to the two or more electrodes.

In some embodiments, the determining step includes solving, via a mathematical solver, using the two response signals or values thereof at a plurality of frequencies acquired via the impedance spectroscopy device, a resistance value and a capacitance value of the first cell side, a resistance value and a capacitance value of the second cell side, a first resistance value and a second resistance value respective solutions in contact with the first and second cell side, and a resistance value across both the first and second cell side, wherein the at least two response signals or values thereof include (i) a first voltage measure across the first or the second cell side between the first electrode and the second electrode and (ii) a second voltage measure across both the first and the second cell side between the first electrode and the third electrode.

In some embodiments, the determining step includes solving, via a mathematical solver, using the response signal or values thereof at a plurality of frequencies acquired via the impedance spectroscopy device, a resistance value and a capacitance value associated with the first cell side, a resistance value and a capacitance value associated with the second cell side, and a first resistance value and a second resistance value respective solutions in contact with the first and second cell side, wherein the response signal or values thereof include a voltage measure across the first electrode and the second electrode.

In another aspect, a method is disclosed comprising: culturing or placing a sample in a measurement chamber; introducing a therapeutic agent, chemical agent, biological agent (viral, bacterial, fungi, etc.), compound (e.g., toxins), and/or environmental stimuli (heating, cooling), to the cell sample; and interrogating two or more electrodes positioned in extracellular spaces of the sample and an electrode placed in an intracellular space of the sample, to provide two or more response signals of the cell sample, wherein the two or more response signals are made available to be assessed by an impedance model of extracellular electrophysiology of a cell for electrical characteristic values for the cell sample, wherein the impedance model includes at least a resistance and capacitance characteristics of the first cell side, a resistance and capacitance characteristics of the second cell side, resistance characteristics of solutions in contact with the first and second cell side, and a resistance characteristic across the first and second cell side, and wherein one or more of the electrical characteristic values are made accessible (e.g., via a graphical user interface or report) for use in a non-invasive characterization of the cell sample in response to a stimulus (e.g., therapeutics or environmental stimulus) applied to the cell sample or non-invasive characterization of the stimulus, wherein the stimulus comprises at least one of a therapeutic agent, disease agent, or compound.

In another aspect, a method is disclosed comprising: culturing or placing a sample in a measurement chamber; introducing a therapeutic agent, chemical agent, biological agent (viral, bacterial, fungi, etc.), compound (e.g., toxins), and/or environmental stimuli (heating, cooling), to the cell sample; and interrogating two or more electrodes positioned in extracellular spaces of the sample to provide a response signal of the cell sample, wherein the response signal is made available to be assessed by an impedance model of extracellular electrophysiology of a cell for electrical characteristic values and a time-constant impedance-associated ratio (tau ratio) for the cell sample, wherein the impedance model includes at least a resistance and capacitance characteristics of the first cell side, a resistance and capacitance characteristics of the second cell side, and wherein one or more of the electrical characteristic values and the time-constant impedance-associated ratio (tau ratio) are made accessible (e.g., via a graphical user interface or report) for use in a non-invasive characterization of the cell sample in response to a stimulus (e.g., therapeutics or environmental stimulus) applied to the cell sample or non-invasive characterization of the stimulus, wherein the stimulus comprises at least one of a therapeutic agent, disease agent, or compound.

In some embodiments, the first cell side is in contact with a first nutrient solution bath (e.g., salt bath), and wherein the second cell side is in contact with a second nutrient solution bath (e.g., salt bath), and wherein the first salt solution and the second salt solution are the same.

In some embodiments, the impedance model of extracellular electrophysiology of the cell assumes the first salt solution and the second salt solution have the same resistance value.

In some embodiments, the impedance model of extracellular electrophysiology of the cell assumes the first salt solution and the second salt solution have different resistance values.

In some embodiments, the first cell side is in contact with a first nutrient solution bath, and wherein the second cell side is in contact with a second nutrient solution bath, wherein the first salt solution and the second salt solution are different.

In some embodiments, the first electrode is operably coupled to the first salt solution bath, and wherein the second electrode is operably coupled to an intracellular cellular space defined between the first cell side and the second cell side.

In some embodiments, the cell sample comprises a cell tissue.

In some embodiments, trends for the one or more of the electrical characteristic values are made accessible for use in the characterization of the cell sample or a stimulus (e.g., therapeutics or environmental stimulus) applied to the cell sample.

In some embodiments, the therapeutic agent, chemical agent, biological agent (viral, bacterial, fungi, etc.), compound (e.g., toxins), and/or environmental stimuli (heating, cooling) are applied once, and a plurality of electrical characteristic values are measured for a pre-defined period of time or until equilibrium is observed in the measurement of the cell culture.

In some embodiments, the therapeutic agent, biological agent (viral, bacterial, fungi, etc.), compound (e.g., toxins), and/or environmental stimuli (heating, cooling) are applied in an on-going basis (e.g., for a period of time), and a plurality of electrical characteristic values are measured for a pre-defined period of time or until equilibrium is observed in the measurement of the cell culture.

In some embodiments, the method further includes monitoring, via a control unit, the one or more of the electrical characteristic values; and triggering the introduction of a therapeutic agent, a biological agent (viral, bacterial, fungi, etc.), a compound (e.g., toxins), and/or environmental stimuli (heating, cooling) based on the monitored one or more of the electrical characteristic values (e.g., exceeding or meeting a pre-defined threshold or range, e.g., user-definable).

In another aspect, a non-transitory computer readable medium having instructions stored thereon, wherein execution of the instructions by a processor causes the processor to operate any one of the above-discussed systems or any one of the above-discussed methods.

Other systems, methods, features, and/or advantages will be or may become apparent to one with skill in the art upon examination of the following drawings and detailed description. It is intended that all such additional systems, methods, features, and/or advantages be included within this description and be protected by the accompanying claims.

### BRIEF DESCRIPTION OF DRAWINGS

The components in the drawings are not necessarily to scale relative to each other. Like reference, numerals designate corresponding parts throughout the several views.

FIGS. 1A-1C each shows an example system 100 (shown as 100a, 100b, 100c) having an analysis system 102 (shown as 102a, 102b, and 102c, respectively) configured to determine epithelial cell membrane properties 104 by fitting measurements acquired from an impedance spectroscopy instrument 108 to an intracellular circuit model 110 in accordance with an illustrative embodiment.

FIGS. 2A-2H depict exemplary methods which can be implemented with any one of the exemplary systems 100a, 100b, or 100c.

FIGS. 3A, 3B, and 3C each shows mathematical models for use by the systems of FIGS. 1A-1C in accordance with an illustrative embodiment.

FIGS. 3D-3F show example methods of operation using the tau ratio for the assessment of a stimulus applied to the cell/tissue sample, e.g., in the system of FIG. 1C, in accordance with an illustrative embodiment.

FIGS. 4A and 4B each depicts an exemplary measurement device comprising a measurement chamber for use by the systems of FIGS. 1A-1C in accordance with an illustrative embodiment.

FIGS. 4C, 4D, and 4E each show an example of an impedance spectroscopy instrument for use by the systems of FIGS. 1A-1C in accordance with an illustrative embodiment.

FIGS. 5A-5C show models of ion transport in epithelia employed in a study conducted to develop and validate a mathematical model for decoding epithelial apical, basolateral, and paracellular electrical properties. FIG. 5D shows the exemplary method of the study being used to determine the electrical transport parameters of intact epithelial tissues.

FIGS. 6A-6E show experimental results conducted in a study to biologically validate mathematical model for decoding epithelial apical, basolateral, and paracellular electrical properties.

FIGS. 7A-7N show additional experimental results conducted in the study to biologically validate mathematical model for decoding epithelial apical, basolateral, and paracellular electrical properties.

FIGS. 8A-8G show a study conducted to develop a mathematical model to measure apical and basolateral membrane properties using extracellular electrochemical impedance spectroscopy.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

Some references are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in a list. All references cited and discussed in this specification are incorporated herein by reference.

### Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces bacterial growth” means reducing the rate of growth of a bacteria relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, chickens, ducks, geese, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

### Example System

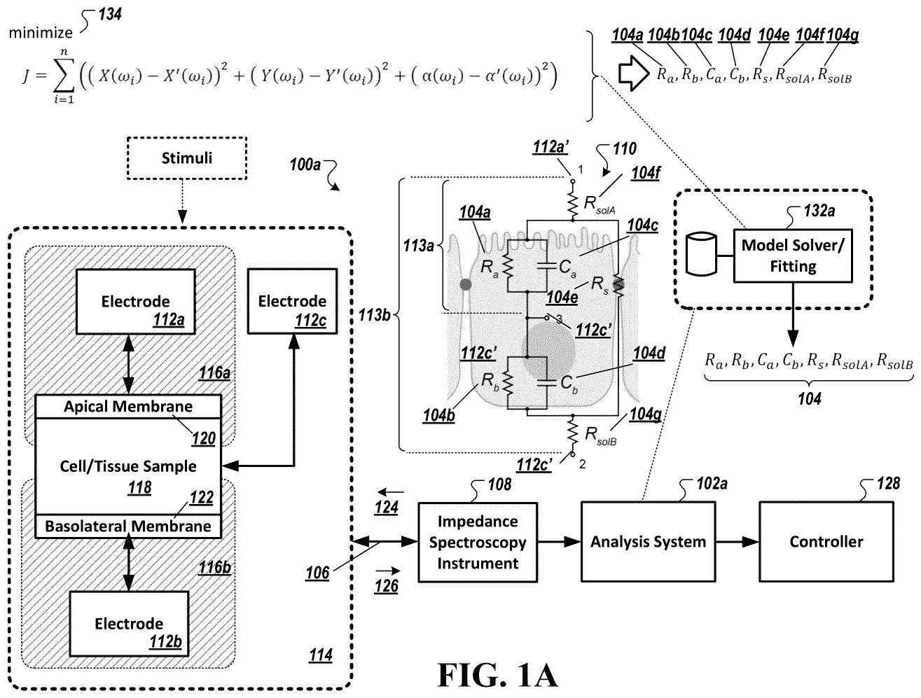

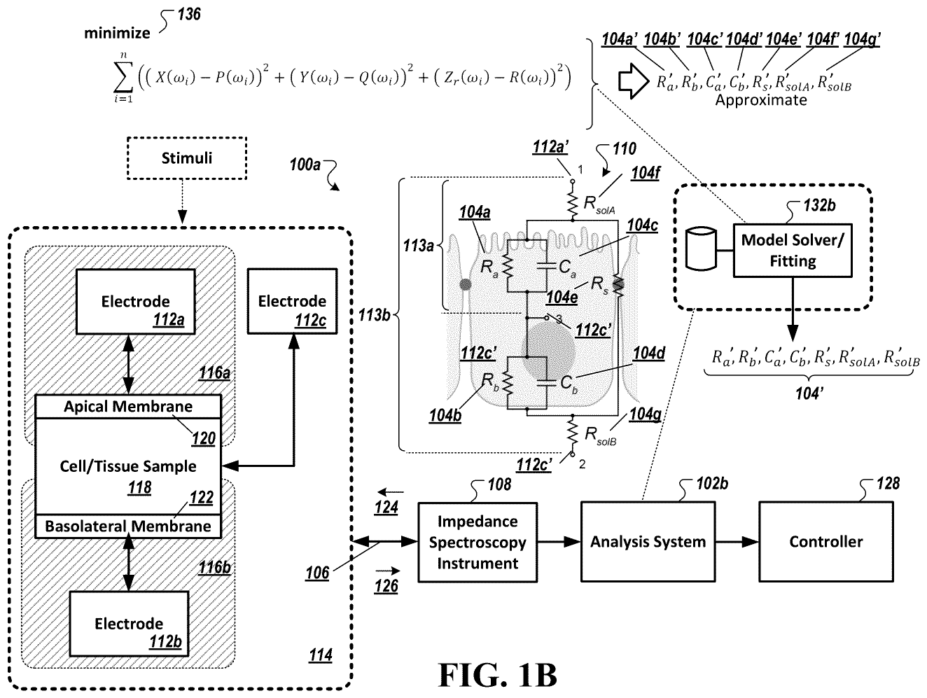

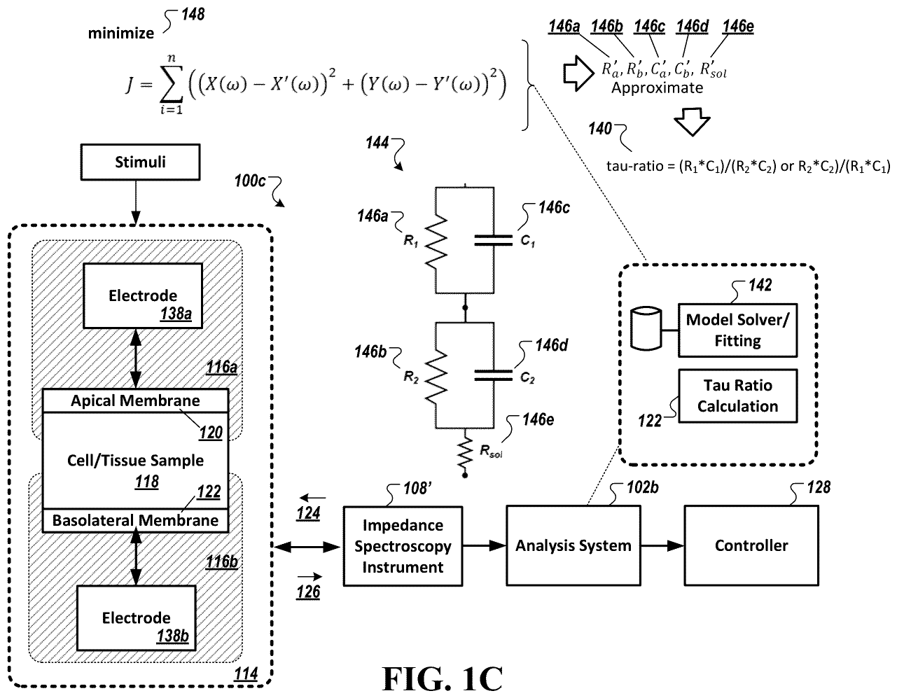

FIGS. 1A-1C each shows an example system 100 (shown as 100a, 100b, 100c) having an analysis system 102 (shown as 102a, 102b, and 102c, respectively) configured to determine epithelial cell membrane properties 104 by fitting measurements acquired from an impedance spectroscopy instrument 108 to an intracellular circuit model 110 in accordance with an illustrative embodiment. Though the analysis systems 100a, 100b, 100c are described in relation to the application of a current and the recording of a voltage, it can also operate via the application of a voltage and the recordation of a current. The impedance spectroscopy instrument may include a current clamp circuit (e.g., galvanostatic) or a voltage clamp circuit (e.g., potentiostatic).

In the example shown in FIG. 1A, the impedance spectroscopy instrument 108 is coupled to a three-electrode set 112 placed in a measurement chamber 114, the electrode set 112 comprising (i) two external/extracellular electrodes 112a, 112b positioned in contact with solvent baths 116 (shown as 116a, 116b) to which the cell/tissue sample 118 is placed and (ii) an internal electrode 112c positioned within the cell/tissue 118. The three electrodes (112a, 112b, 112c) provide two voltage measurements across a set of frequencies in which one measurement (113a) is acquired between the two external electrodes (112a, 112b; shown as nodes 112a′, 112b′) and the second measurement (113b), as a voltage divide, is acquired between the internal electrode 112c (shown as node “3” 112c′) and one of the external electrode (e.g., 112a or 112b). In the example shown in FIG. 1A, the cell/tissue sample 118 has an apical membrane region 120 and a basolateral membrane region 122 in contact with the solvent bath 116a, 116b.

The term “apical membrane” refers to a cellular membrane that faces towards an external environment (e.g., lumen) in an in-situ context, whereas the term “basolateral membrane,” interchangeably used with the term “basal,” refers to a cellular membrane that faces away from an external environment in an in-situ context. In an Ussing chamber or the like, the apical membrane and the basolateral membrane would be in a simulated environment, e.g., mimicking the body conditions, though not necessarily for the purpose of a test. Cell/tissue samples having an apical membrane and a basolateral membrane are typically barrier tissues, and in some embodiments, can include epithelial cells (e.g., retinal pigment epithelium, bronchiole epithelium, etc.), endothelial cells, mesenchymal tissues, or any other barrier tissue.

The intracellular circuit model 110 includes equivalent electrical circuit components of the apical pathways (shown as a parallel circuit having resistance Rₐ (104a) and capacitance Cₐ (104c)), the basolateral pathways (shown as a parallel circuit having resistance R_{b} (104b) and capacitance C_{b} (104d)), the paracellular (shunt) pathway (having a shunt resistance Rₛ (104e) in parallel to the apical and basolateral pathways, and the surrounding media and growth substrates (having a solution resistance R_{solA} (104f) and R_{solB} (104g) for each side of the membrane).

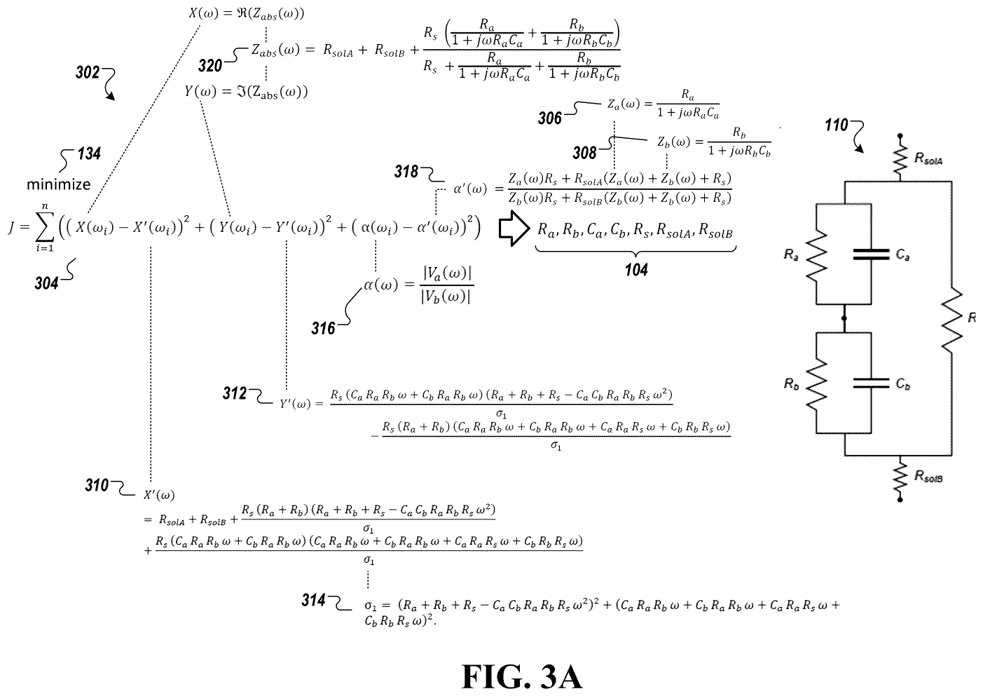

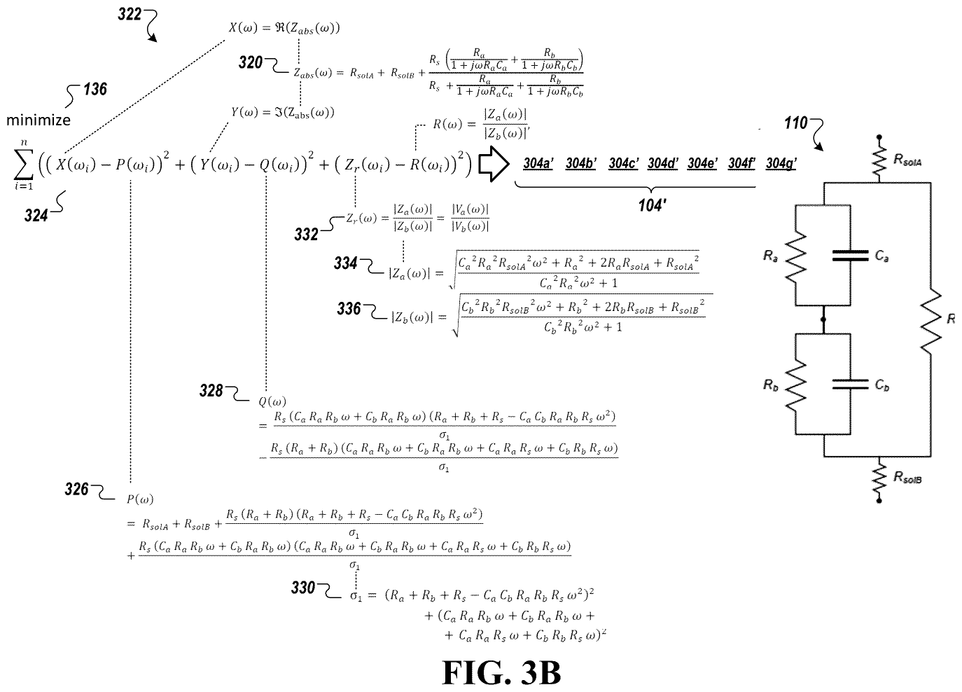

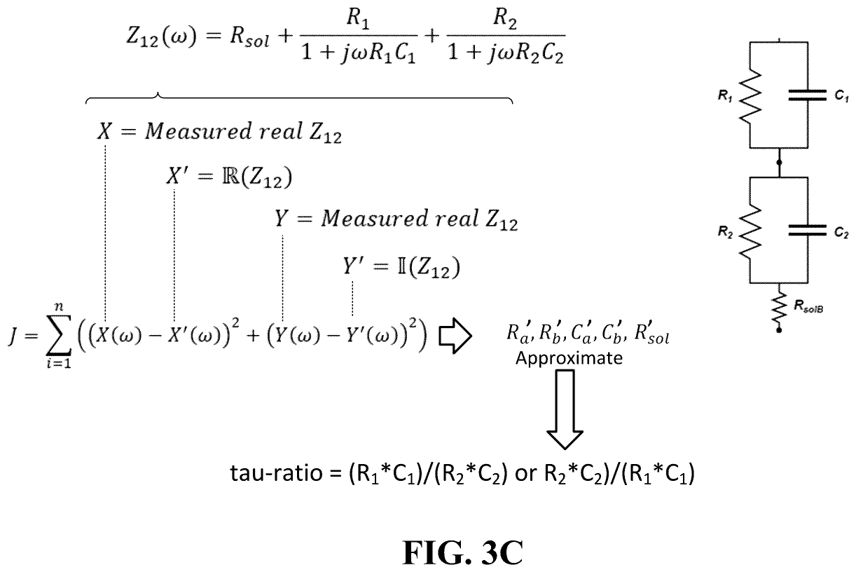

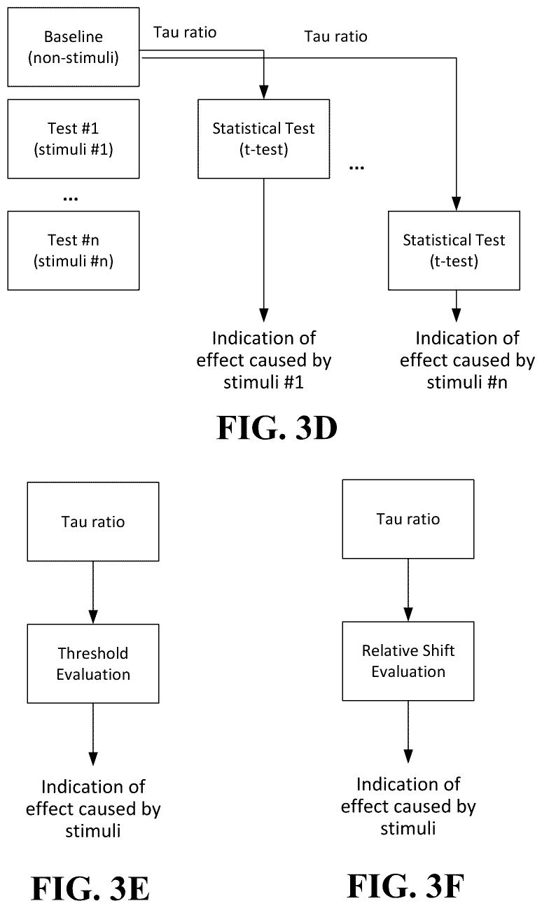

The analysis system 102a includes a model solver 132 (shown as 132a) that is configured to fit the measured signals 126 across the different frequencies to the equivalent electrical circuit components (i.e., 104a, 104b, 104c, 104d, 104e, 104f, 104g). In the example shown in FIG. 1A, the model solver 132a is configured to fit the measurements 126 to the circuit components of the model 110 via a minimization operation 134 to determine the equivalent electrical circuit components 104a-104g corresponding to the epithelial cell membrane properties 104 of the cell/tissue sample 118, including the resistance of the apical pathway Rₐ (104a), the capacitance of the apical pathway Cₐ (104b), the resistance of the basolateral pathway R_{b} (104c), the capacitance of the basolateral pathway C_{b} (104d), the resistance Rₛ (104e) of the paracellular (shunt) pathway, the resistance R_{solA} (104f) of the surrounding media and growth substrate on the apical side, and the resistance R_{solB} (104g) of the surrounding media and growth substrate on the basolateral side. The model can be characterized as having a parallel shunt pathway and a parallel RC circuit for the apical and basolateral membranes. FIG. 3A shows the mathematical model 302 for the minimization operation 134.

Impedance Ratio Analysis System. The analysis system 102a may be an edge device that is operatively coupled to the impedance spectroscopy instrument 108. In other embodiments, the analysis system 102a is a cloud infrastructure that interfaces with the impedance spectroscopy instrument 108 over a network or a data store that operates with the impedance spectroscopy instrument 108.

The impedance spectroscopy instrument 108 is configured to generate two or more interrogation signals 124 that may each include a varying sweep of AC frequencies. In some embodiments, the impedance spectroscopy instrument 112 is configured to output the interrogation signals 148 having waveforms for a set of frequency bands. The interrogation signals 124, e.g., as current signals, are applied between the two extracellular electrodes 112a, 112b and between an intracellular electrode 112c and one of the extracellular electrodes 112a, 112b. The cell/tissue sample would provide corresponding response signals 126, e.g., as a voltage. The response signals 126 are recorded by the impedance spectroscopy instrument 112 and provided to the controller 128. The response signals 126 can be sent to data storage (e.g., 132) and are later retrieved by the analysis system.

Approximate Impedance Analysis System: FIG. 1B shows an example system 100b having an analysis system 102b configured to determine approximate epithelial cell membrane properties 104′ by fitting measurements acquired from an impedance spectroscopy instrument 108 to an intracellular circuit model 110 in accordance with another illustrative embodiment. In the example shown in FIG. 1B, the impedance spectroscopy instrument 108 is also coupled to a three-electrode set 112 placed in a measurement chamber 114, the electrode set 112 comprising (i) two external/extracellular electrodes 112a, 112b positioned in contact with solvent baths 116 (shown as 116a, 116b) to which the cell/tissue sample 118 is placed and (ii) an internal electrode 112c positioned within the cell/tissue 118.

The analysis system 102b includes a model solver 132 (shown as 132b) that is configured to fit the measured signals 126 across the different frequencies to the equivalent electrical circuit components (i.e., 124a, 124b, 126a, 126b, 128, 130a, 130b). In the example shown in FIG. 1B, the model solver 132b is configured to fit the measurements 126 to the circuit components of the model 110 via a curvefit operation 136 to determine the approximate equivalent electrical circuit components 104a-104g corresponding to the epithelial cell membrane properties 104 of the cell/tissue sample 118, including the approximate resistance of the apical pathway R′ₐ(104a′), the approximate capacitance of the apical pathway C′ₐ (104b′), the approximate resistance of the basolateral pathway R′_{b} (104c′), the approximate capacitance of the basolateral pathway C′_{b} (104d′), the approximate resistance R′ₛ (104e′) of the paracellular (shunt) pathway, the approximate resistance R_{solA}′ (104f′) of the surrounding media and growth substrate on the apical side, and the approximate resistance R_{solB′} (104g′) of the surrounding media and growth substrate on the basolateral side. FIG. 3B shows the mathematical model 304 for the curvefit operation 136. The approximate solution 104a′-104′g is determined based on a constraint that assumes the electrical characteristics of the surrounding media and growth substrate to be the same.

Time-Constant Impedance Ratio Analysis System. FIG. 1C shows an example system 100c having an analysis system 102c configured to determine non-invasively the polarization of epithelial cells in response to stimuli, as a time-constant impedance-associated ratio (also referred to as “tau ratio”) 140, by (i) fitting (shown as minimize operation 148) extracellular measurements acquired from an impedance spectroscopy instrument 108 to an extracellular circuit model 144 and (ii) using the elements in the circuit model in a time-constant impedance-associated ratio model. Notably, the time-constant impedance-associated ratio (“tau ratio”) 140 is a parameter that can be determined from two electrodes (e.g., 138a, 138b) positioned for extracellular measurements. The analysis can be employed as a quick and lower or low-resolution evaluation/monitoring of membrane-specific parameters in epithelia, e.g., in a massively parallel screening system, e.g., for drug discovery performed in a non-invasive manner.

In FIGS. 1A and 1B, the three electrode systems 100a, 100b each entail inserting a microelectrode into the cytoplasm of an epithelial cell and the same extracellular electrodes used in the tau ratio. As discussed, measuring the three-electrode system and fitting the measurement to the circuit model 110 via mathematical operations 134 or 136 would provide a resistance parameter for the shunt pathway that allows the direct calculation of each membrane parameter.

Referring still to FIG. 1C, the time-constant impedance-associated ratio (“tau ratio”) 140 can be readily measured in a high throughput manner using measurements in the measurement chambers that do not require insertion of an electrode into the cell to provide a measure of the polarization of the epithelial cells in response to a therapeutic or environmental stimulus that can be used in an evaluation the therapeutic or environmental stimulus. The two-electrode screening system may be used in combination with the three-electrode system to provide an initial screen operation to which the more accurate but low throughput three-electrode system can be used in a verify operation (e.g., ground truth).

In the example shown in FIG. 1C, the impedance spectroscopy instrument 108 is coupled to a two-electrode set 138 placed in a measurement chamber 114, the electrode set 138 comprising two external/extracellular electrodes 138a, 138b positioned in contact with solvent baths 116 (shown as 116a, 116b), growth substrate, or other substrate described or referenced herein, to which the cell/tissue sample 118 is placed or embedded.

The analysis system 102c includes a model solver 142 that is configured to fit the measured signals 126 across the different frequencies to circuit model 144 to elements 146a-146f of the circuit model 144. The elements 146a-146e are then combined to determine the time-constant impedance-associated ratio (“tau ratio”) 140.

In the example shown in FIG. 1C, the model solver 142 is configured to fit the measurements 126 to the circuit components 146a-146g of the model 144 via a curvefit operation 148 to determine the equivalent electrical circuit components 146a-146e having some correspondence to the epithelial cell membrane properties 104 of the cell/tissue sample 118, including a first parallel resistance element 146a, a second parallel resistance element 146c, a first parallel capacitance element 146b, a second parallel capacitance element 146d, and a series circuit element 146e. FIG. 3C shows several mathematical models to which the minimize operation 148 can be applied.

The tau ratio, obtained from extracellular measurements of single-layer barrier cells, can provide unique insights compared to conventional extracellular trans-epithelial resistance (TER) and trans-epithelial potential (TEP) measurements. In some embodiments, the tau ratio can be used for: enhanced quality control serving as a cell health indicator. Variations from the standard tau ratio may pinpoint potential abnormalities, offering a more sensitive and specific health assessment than that of TER and TEP can typically detect. This precision can be particularly valuable for quality control in epithelial-based therapies.

In some embodiments, the tau ratio can be used for a comprehensive response analysis in which the tau ratio is used to uniquely measure the relative shifts between cell membrane resistances and capacitances in response to external stimuli, a capability not directly addressed by TER and TEP. While it doesn't isolate changes in the apical or basolateral membranes, it can reveal the overall interplay and balance between them. This perspective is crucial for understanding complex cellular responses and interactions that traditional measurements might overlook.

Examples of external stimulus include (i) changing ion composition of external media (e.g., reducing extracellular potassium concentration), (ii) adding or removing chemicals (e.g., adding extracellular adenosine tri-phosphate), (iii) adding extracellular molecules/cells (e.g., adding photoreceptor outer segments, in vitro).

To this end, the tau ratio analysis system of FIG. 1C can enrich the toolkit of cellular analysis, providing insights that are not readily accessible through classical electrophysiology assays.

### Example Method of Operation

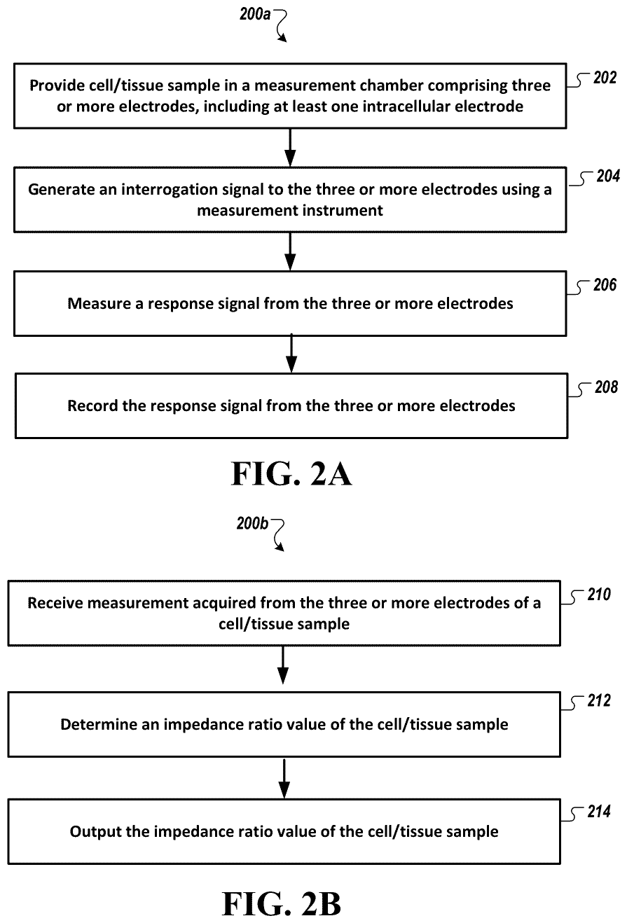

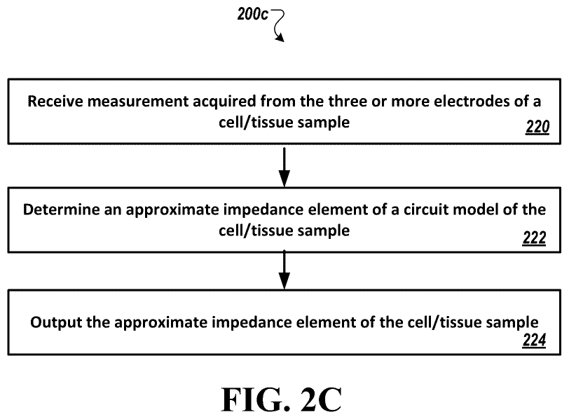

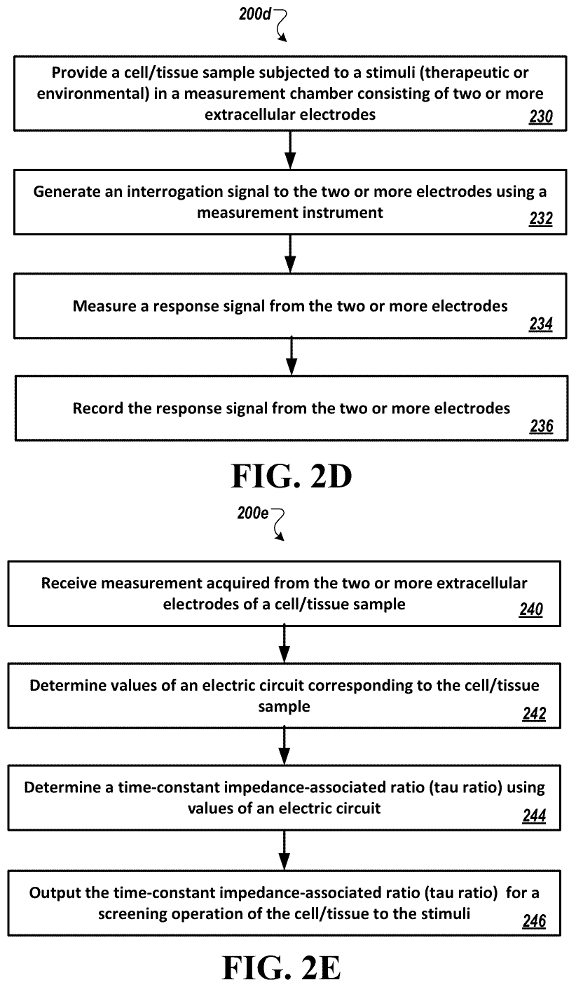

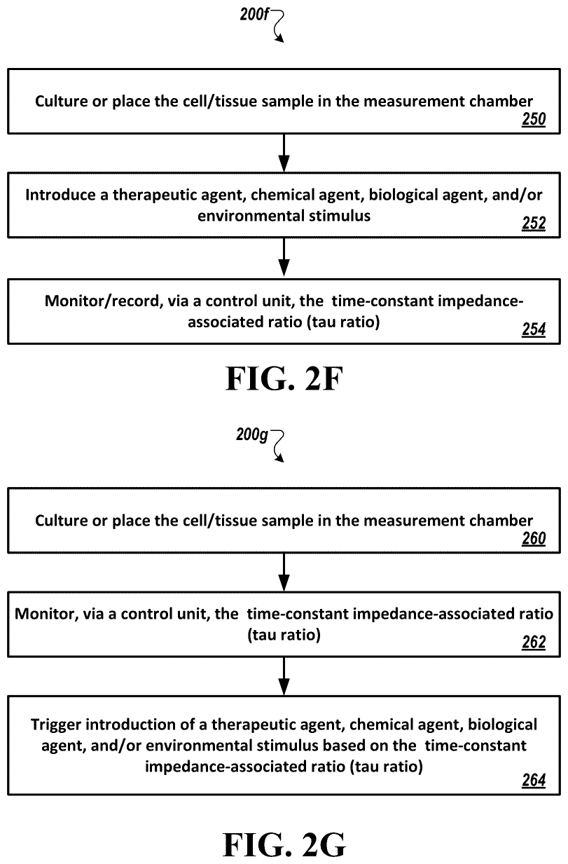

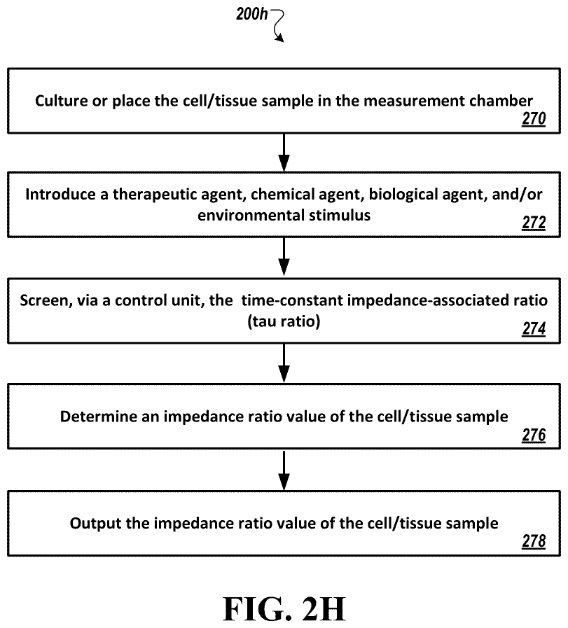

FIGS. 2A-2H depict exemplary methods which can be implemented with any one of the exemplary systems 100a, 100b, or 100c.

Impedance Ratio Analysis. FIGS. 2A and 2B shows an exemplary method 200a, 200b of system 100a. The exemplary method 200a includes providing (202) a cell/tissue sample (e.g., 118) in a measurement chamber (e.g., 114) comprising three or more electrodes (e.g., 112), including at least one intracellular electrode, generating (204) an interrogation signal (e.g., 124) to the three or more electrodes (e.g., 112) using a measurement instrument (e.g., 108), measuring (206) a response signal (e.g., 126) from the three or more electrodes (e.g., 112a), and recording (208) the response signal (e.g., 126) for subsequent analysis.

FIG. 2B shows the method 200b to perform the subsequent analysis. The method 200b includes receiving (210) the measurement acquired from the three or more electrodes of a cell/tissue sample (e.g., 118), determining (212) an impedance ratio value, e.g., as electrical characteristic values 104, of the cell/tissue sample (e.g., 118), and outputting (214) impedance ratio value of the cell/tissue sample (e.g., 118). In some embodiments, the outputting operation entails storing the determined impedance ratio value to be made accessible by an external computing device (e.g., to output to a user in a graphic user interface or in a report).

The determination operation (212) may include performing the minimization operation 134, e.g., via a fitting software/algorithm, to determine the equivalent electrical circuit components 104a-104g of a circuit model (e.g., 110) corresponding to the epithelial cell membrane properties 104 of the cell/tissue sample 118, including the resistance of the apical pathway Rₐ (104a), the capacitance of the apical pathway Cₐ (104b), the resistance of the basolateral pathway R_{b} (104c), the capacitance of the basolateral pathway C_{b} (104d), the resistance Rₛ (104e) of the paracellular (shunt) pathway, the resistance R_{solA} (104f) of the surrounding media and growth substrate on the apical side, and the resistance R_{solB} (104g) of the surrounding media and growth substrate on the basolateral side. Further description of the circuit model 110 and analysis (e.g., performing step 212) is provided in relation to FIG. 3A.

Appropriate Impedance Analysis. FIG. 2C shows an exemplary computer-implemented method 200b, e.g., of system 100b to determine the approximate epithelial cell membrane properties 104′ of a cell/tissue sample 118. The approximate properties 104′ have higher discrepancies in general as compared to the analysis described in relation to FIG. 2A. In certain configurations, e.g., where the nutrient or solution baths have similar characteristics, the accuracy is higher.

The method 200c includes receiving (220) measurement acquired, e.g., via operation 200a, from the three or more electrodes of a cell/tissue sample, determining (222) an approximate impedance element of a circuit model of the cell/tissue sample, and outputting (224) the approximate impedance element of the cell/tissue sample.

Additional description of the method 200b may be found in Lewallen, Colby, Epithelial Electrophysiology Using Intracellular Robotics and Extracellular Impedance Spectroscopy (thesis), 2022, http://hdl.handle.net/1853/66173.

Time-Constant Impedance Ratio Analysis. FIG. 2D shows an exemplary computer-implemented method 200c, e.g., of system 100d providing (220) a cell/tissue sample (e.g., 118) in a measurement chamber (e.g., 114) consisting only of two or more electrodes (e.g., 138), generating (232) an interrogation signal (e.g., 124) to the two or more electrodes (e.g., 138) using a measurement instrument (e.g., 108), measuring (234) a response signal (e.g., 126) from the two or more electrodes (e.g., 112a), and recording (236) the response signal (e.g., 126) for subsequent analysis.

FIG. 2E shows the method 200e to perform the subsequent analysis. The method 200e includes receiving (240) the measurement acquired from the two or more electrodes of a cell/tissue sample (e.g., 118), determining (242) the value of an electric circuit model (e.g., extracellular circuit model) corresponding to the cell/tissue sample (e.g., 118), and determining (244) a time-constant impedance-associated ratio model corresponding to the polarization of epithelial cells in response to stimuli.

The time-constant impedance-associated ratio model (Tau-Ratio) employs a simplified model with only two parallel RC circuits, in series, representing (but not equal to) the apical and basolateral membranes (without the shunt pathway), among other models described herein. FIG. 3C shows different extracellular models to which an approximate value of an electric circuit model can be determined via a fitting operation. After fitting the data to the simplified electronic circuit, a unique mathematical combination of the parameters is employed to determine the time-constant impedance-associated ratio (tau ratio) as a measure of polarization of epithelial cells in response to stimuli. The model provides an approximate ratio of the apical and basolateral membrane time constants, offering a general indicator of membrane polarization, especially in response to external stimulus.

Impedance is the sum of a real (resistance) and imaginary (reactance) component. An example set of steps of the measurement include applying a current or voltage stimulus across a tissue, and recording the amplitude and phase of the response signal (if sending current, record voltage and vice versa) using a computer device. For example, a custom FFT module, or (commercially) a Metrohm Autolab, or Digilent discover. The amplitude and phase of the response signal are typically converted to impedance (aka resistance and reactance), e.g., in a measurement device but could be done in user-created algorithms and some simple math. The process then includes fitting the measured impedance to a mathematical model of RPE comprising two parallel RC filters in series with each other. The tau-ratio and TER can then be calculated from the magnitude of the values in the model after fitting. Specifically, TER=R1+R2 and tau-ratio=R1*C1/(R2*C2) or R2*C2/(R1*C1). The selection for the ratio may be user-dependent. Either the user can use prior knowledge to select the equation or they can enforce the ratio is greater than or less than 1.

Notably, the time-constant impedance-associated ratio (tau ratio) (e.g., 140) can be determined from two electrodes (e.g., 138a, 138b) positioned for extracellular measurements to provide insight into a stimuli, therapeutic or environmental, that is applied to a cell/tissue sample (e.g., 118).

Monitoring Method. FIG. 2F shows a method 200f of using the tau ratio in the evaluation of a disease model. The method 200f includes culturing or placing (250) a cell/tissue sample (e.g., 118) in a measurement chamber (e.g., 114), introducing (252) a therapeutic agent, chemical agent, biological agent, and/or environmental stimulus, and monitoring (254), via a control unit, the tau ratio to determine presence or non-presence of an effect of the stimuli to the cell/tissue sample (e.g., 118).

In some embodiments, the tau ratio can be used for anomaly detection via application to a statistic tool. FIG. 3D shows an example application of the tau ratio as an anomaly detector. In the example shown in FIG. 3D, measurements can be taken from a set of cell/tissue sample, each having different stimuli applied to them. A baseline measurement can also be acquired. Tau ratio assessment can be acquired from each of the tests and used as a basis for the statistical test (e.g., T-test, ANOVA test, chi-test, etc.) to determine if the stimuli cause a statistical difference in measurement to the baseline.

In some embodiments, the tau ratio can be used for enhanced quality control. FIG. 3E shows an example application of the tau ratio in quality control assessment. As shown in FIG. 3E, the tau ratio can be compared to a threshold value to provide an indicator of cell heath. To this end, the tau ratio can act as a cell health indicator. In some embodiments, variations from the standard tau ratio (e.g., 3.1±0.4 or 11±2) may pinpoint potential abnormalities, offering a more sensitive and specific health assessment than what TER and TEP can typically detect. This precision would be particularly valuable for quality control in epithelial-based therapies.

In some embodiments, the tau ratio can be used for a comprehensive response analysis to measure relative shifts between cell membrane resistances and capacitances in response to external stimuli. As shown in FIG. 3F, the tau ratio is applied to a relative shift analysis to measure the relative shifts between cell membrane resistances and capacitances in response to external stimuli, a capability not directly addressed by TER and TEP, to provide an assessment of the overall interplay and balance between the apical and basolateral membrane. The evaluation can provide an output that can be used in the understanding of complex cellular responses.

As discussed above, examples of external stimulus may include but are not limited to (i) changing ion composition of external media (e.g., reducing extracellular potassium concentration), (ii) adding or removing chemicals (e.g., adding extracellular adenosine tri-phosphate), (iii) adding extracellular molecules/cells (e.g., adding photoreceptor outer segments, in vitro).

Additional examples of external media, chemicals, extracellular molecules/cells may include therapeutic agent, chemical agent, biological agent, and/or environmental stimuli can include viral, bacterial, fungi, toxins, heating, and cooling.

Particular screening applications disclosed herein relate to the testing of pharmaceutical compounds in drug research (In Vitro Methods in Pharmaceutical Research”, Academic Press, 1997, and U.S. Pat. No. 5,030,015). Assessment of the activity of candidate pharmaceutical compounds generally involves administering a candidate compound, determining any change in the morphology, marker phenotype and expression, or metabolic activity of the cells and function of the cells that is attributable to the compound (compared with untreated cells or cells treated with an inert compound), and then correlating the effect of the compound with the observed change.

The screening may be done, for example, either because the compound is designed to have a pharmacological effect on certain cell types, or because a compound designed to have effects elsewhere may have unintended side effects. Two or more drugs can be tested in combination (by combining with the cells either simultaneously or sequentially), to detect possible drug-drug interaction effects. In some applications, compounds are screened initially for potential toxicity (Castell et al., pp. 375-410 in “In vitro Methods in Pharmaceutical Research,” Academic Press, 1997). Cytotoxicity can be determined in the first instance by the effect on cell viability, survival, morphology, and expression or release of certain markers, receptors or enzymes. Effects of a drug on chromosomal DNA can be especially at unscheduled times in the cell cycle, or above the level required for cell replication, is consistent with a drug effect. Unwanted effects can also include unusual rates of sister chromatid exchange, determined by metaphase spread. The reader is referred to A. Vickers (PP 375-410 in “In vitro Methods in Pharmaceutical Research,” Academic Press, 1997) for further elaboration.

Examples of methods include, but are not limited to, the standard textbook In vitro Methods in Pharmaceutical Research, Academic Press, 1997 and U.S. Pat. No. 5,030,015. Assessment of the activity of candidate pharmaceutical compounds generally involves combining the cells with the candidate compound, either alone or in combination with other drugs. The investigator determines any change in the morphology, marker phenotype, or functional activity of the cells that is attributable to the compound (compared with untreated cells or cells treated with an inert compound), and then correlates the effect of the compound with the observed change.

An agent that inhibits the formation of 11 spectrin fragment formation may be formulated as a pharmaceutical composition or medicament. Pharmaceutical compositions adapted for direct administration include, without limitation, lyophilized powders or aqueous or non-aqueous sterile injectable solutions or suspensions, which may further contain antioxidants, buffers, bacteriostats and solutes that render the compositions substantially isotonic with the blood of an intended recipient. Other components that may be present in such compositions include water, alcohols, polyols, glycerin and vegetable oils, for example. Extemporaneous injection solutions and suspensions may be prepared from sterile powders, granules and tablets. The agents may be supplied, for example, but not by way of limitation, as a lyophilized powder, which is reconstituted with sterile water or saline prior to administration to the patient.

Pharmaceutical compositions may comprise a pharmaceutically acceptable carrier. Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy) propyl)N,N,N-trimethylammonium chloride (DOTMA), diolesylphosphotidyl-ethanolamine (DOPE), and liposomes. Such compositions should contain a therapeutically effective amount of the compound, together with a suitable amount of carrier so as to provide the form for direct administration to the patient.

The composition may be in the form of a pharmaceutically acceptable salt which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Exemplary target molecules include, but are not limited to free ADPR, adenosine monophosphate (AMP), adenosine diphosphate (ADP), adenosine triphosphate (ATP), cyclicAMP (cAMP), guanosine monophosphate (GMP), guanosine diphosphate (GDP), guanosine triphosphate (GTP), cyclic GMP (cGMP), inositol triphosphate (IP3), diacylglycerol (DAG), calcium (Ca²⁺), epinephrine, norepinephrine, acetylcholine, histamine, estrogen, testosterone, progesterone, cholesterol, corticosteroids, thyroid hormone, vitamin D, retinoic acid, nitric oxide (NO), carbon monoxide (CO), glutamate, dopamine, serotonin, glycine, gamma-aminobutyric acid (GABA), insulin, glucagon, and other signaling molecules. In some embodiments, the target molecule comprises a carbohydrate or sugar molecule, or derivatives thereof. Exemplary sugar molecules include, but are not limited to ribose, arabinose, glucose, fructose, sucrose, cellulose, galactose, lactose, maltose, starch, glycogen, dextrose, fucose, inositol, maltodextrin, mannose, ribulose, trehalose, xylose, and derivatives and isomers thereof.

In some embodiments, the target molecule comprises a nucleotide, or derivatives thereof. Exemplary nucleotides include, but are not limited to adenine, thymine, cytosine, guanine, uracil, 5-bromouracil, hypoxanthine, and derivatives and analogues thereof.

In some embodiments, the target molecule comprises a metabolic intermediate. As used herein, a metabolic intermediate refers to molecules that are precursors or metabolites of biologically active molecules. It should be noted that metabolic intermediates may have minor importance to cellular function, but they are important regulators for enzyme functions. Exemplary metabolic intermediates include, but are not limited to, malate, lactate, gluconate, citrate, oxaloacetate, oxoglutarate, acetyl CoA, fumarate, aconitate, isocitrate, ketoglutarate, succinyl CoA, succinate, pyruvate, nicotinamide adenine dinucleotide (NAD+ or NADH), nicotinamide adenine dinucleotide phosphate (NADP+ or NADPH), flavin adenine dinucleotide (FAD+ or FADH), ubiquinol, ubiquinone, and coenzyme Q.

Control Method. FIG. 2G shows a method 200g of using the tau ratio in the control of the evaluation of a disease model. The method 200g includes culturing or placing (260) a cell/tissue sample (e.g., 118) in a measurement chamber (e.g., 114) and introducing of a first therapeutic agent, chemical agent, biological agent, and/or environmental stimuli to yield a first effect (e.g., damaging, infecting, or weakening the cell/tissue sample), monitoring (262), via a control unit, the tau ratio to determine presence or non-presence of an effect of the stimuli to the cell/tissue sample (e.g., 118), and using the monitoring (e.g., as described in the examples provided in relation to FIGS. 3D, 3E, and 3F) to trigger of a second therapeutic agent, chemical agent, biological agent, and/or environmental stimulus based on the monitored impedance ratio to yield a second effect (e.g., repairing, treating, or strengthening the cell/tissue sample).

Screen/Evaluation Method. FIG. 2H shows a method 200h of using the tau ratio in a screening operation for a full evaluation. The method 200h includes culturing or placing (270) a cell/tissue sample (e.g., 118) in a measurement chamber (e.g., 114), introducing (272) a therapeutic agent, chemical agent, biological agent, and/or environmental stimulus, and screening (274), via a control unit, based on the tau ratio, to determine presence or non-presence of an effect of the stimuli to the cell/tissue sample (e.g., 118). In some embodiments, the determination can be based on examples provided in relation to FIG. 3D, 3E, or 3F).

Method 200h then includes performing the intracellular measurement of the cell/tissue sample, e.g., by inserting an electrode into the cell/tissue sample as described in relation to FIG. 1A. The method 200 then includes measuring the three or more electrodes (e.g., 112a, 112b, 112c) to determine (278) an impedance ratio value, e.g., as electrical characteristic values (e.g., 104), of the cell/tissue sample (e.g., 118).

### Example Intracellular Cell Circuit Model

FIG. 3A shows the mathematical model 302 for the minimization operation 134 that can be performed by the solver (e.g., 132a) to determine the electrical characteristic values 104 for a cell/tissue sample. The minimization operation 134 has the form of Equation 5 (304) provided below. The simplest version of the equations of the model can be solved to fully determine the impedance ratio, per Equations 1-6 provided below.

The mathematical model 302 of the circuit 110 can be used to quantify the electrical properties of epithelia. The trans-epithelial model of impedance is complex and can be represented as the sum of two orthogonal vectors: the real impedance X(ω), and the imaginary impedance, Y(ω). This representation is based on the mathematical concept of complex numbers, where the complex impedance, Z_{abs}(ω), can be expressed as Z_{abs}(ω)−X(ω)+jY(ω) where j represents the imaginary unit (√−1). The representation allows for separate measurements and analysis of the real and imaginary components of the impedance, which can be used to characterize the electrical properties of the epithelial tissue. Additionally, the measured complex impedance, Z_{abs} can be mathematically modeled to provide Equation Set #1 having elements (306, 308) by combining the circuit elements in FIG. 1C such that:

$${Z}_{abs}^{′} ( ω ) = {R}_{solA} + {R}_{solB} + \frac{{R}_{s} ( {Z}_{a} ( ω ) + {Z}_{b} ( ω ) )}{{R}_{s} + {Z}_{a} ( ω ) + {Z}_{b} ( ω )} , where \\ {Z}_{a} ( ω ) = \frac{{R}_{a}}{1 + j ω {R}_{a} {C}_{a}} \\ {Z}_{b} ( ω ) = \frac{{R}_{b}}{1 + j ω {R}_{b} {C}_{b}} \qquad ( Eq . Set \# 1 )$$

Because in Equation Set #1, Z_{abs}′ is complex and can be rewritten in a similar form per Equation Set #2, shown as elements 310, 312, 314 in FIG. 3A.

$${Z}_{abs}^{′} ( ω ) = {X}^{′} ( ω ) + {jY}^{′} ( ω ) , where \\ {X}^{′} ( ω ) = {R}_{solA} + {R}_{solB} + \frac{{R}_{s} ( {R}_{a} + {R}_{b} ) ( {R}_{a} + {R}_{b} + {R}_{s} - {C}_{a} {C}_{b} {R}_{a} {R}_{b} {R}_{s} {ω}^{2} )}{{σ}_{1}} + \frac{\begin{matrix}{R}_{s} ( {C}_{a} {R}_{a} {R}_{b} ω + {C}_{b} {R}_{a} {R}_{b} ω ) \\ ( {C}_{a} {R}_{a} {R}_{b} ω + {C}_{b} {R}_{a} {R}_{b} ω + {C}_{a} {R}_{a} {R}_{s} ω + {C}_{b} {R}_{b} {R}_{s} ω )\end{matrix}}{{σ}_{1}} \qquad ( Eq . Set \# 2 )$$

$${Y}^{′} ( ω ) = \frac{{R}_{s} ( {C}_{a} {R}_{a} {R}_{b} ω + {C}_{b} {R}_{a} {R}_{b} ω ) ( {R}_{a} + {R}_{b} + {R}_{s} - {C}_{a} {C}_{b} {R}_{a} {R}_{b} {R}_{s} {ω}^{2} )}{{σ}_{1}} - \frac{{R}_{s} ( {R}_{a} + {R}_{b} ) ( {C}_{a} {R}_{a} {R}_{b} ω + {C}_{b} {R}_{a} {R}_{b} ω + {C}_{a} {R}_{a} {R}_{s} ω + {C}_{b} {R}_{b} {R}_{s} ω )}{{σ}_{1}} \\ {σ}_{1} = {( {R}_{a} + {R}_{b} + {R}_{s} - {C}_{a} {C}_{b} {R}_{a} {R}_{b} {R}_{s} {ω}^{2} )}^{2} + {( {C}_{a} {R}_{a} {R}_{b} ω + {C}_{b} {R}_{a} {R}_{b} ω + {C}_{a} {R}_{a} {R}_{s} ω + {C}_{b} {R}_{b} {R}_{s} ω )}^{2}$$

In addition to Z_{abs}, the membrane ratio α(a), shown as element 316, can be determined as an independent parameter per Equation 3. The membrane ratio α(a) can be considered as an extension of

$$VDR ( ω = π \frac{rad}{s} ) = \frac{\vert {V}_{a} ( ω ) \vert / \vert {I}_{a} ( ω ) \vert}{\vert {V}_{b} ( ω ) \vert / \vert {I}_{b} ( ω ) \vert} ≅ \frac{{R}_{a}}{{R}_{b}} ,$$

measured at many frequencies, rather than a single, low frequency.

$$a ( ω ) = \frac{\vert {V}_{a} ( ω ) \vert}{\vert {V}_{b} ( ω ) \vert} \qquad ( Eq . 3 )$$

Because the membrane ratio is a ratio of magnitudes, it is not a complex number and has no units. The mathematical model of the membrane ratio (α′), shown as element 318 in FIG. 3A, can be derived as a function of the circuit parameters in FIG. 1A as shown in Equation 4 where Zₐ(ω) and Z_{b}(ω) are defined per Equation Set #1.

$${a}^{′} ( ω ) = \frac{{Z}_{a} ( ω ) {R}_{s} + {R}_{solA} ( {Z}_{a} ( ω ) + {Z}_{b} ( ω ) + {R}_{s} )}{{Z}_{b} ( ω ) {R}_{s} + {R}_{solB} ( {Z}_{a} ( ω ) + {Z}_{b} ( ω ) + {R}_{s} )} \qquad ( Eq . 4 )$$

In Equation 4, when a is very low (e.g., 0.5 Hz), and when R_{solA} and R_{solB} are both much lower than Rₐ and R_{b}, that aᵣ≅VDR. Each circuit element in FIG. 1A can be treated as an element in an array, βk, where the subscript k represents the kth element in the array (e.g., β₁=R_{solA}, β₂=Rₐ, etc.). The measured values for the real impedance X(ω), the imaginary impedance Y(ω), and the membrane ratio a(ω) can be modeled by a unique combination of βₖ to calculate X′(ω), Y′(ω), and a′(ω) per Equation Sets #1, #2 and Equations 3 and 4.

To this end, the electrical properties of a monolayer can be determined by using a nonlinear least squared solver, as described herein, to minimize an objective function, J, which is defined as the sum of the squared residuals, or error, between the measured and modeled data at all frequencies. In mathematical form, J is calculated per Equation 5 (304).

$$J = {\sum }_{i = 1}^{n} ( {( X ( {ω}_{i} ) - {X}^{′} ( {ω}_{i} ) )}^{2} + {( Y ( {ω}_{i} ) - {Y}^{′} ( {ω}_{i} ) )}^{2} + {( a ( {ω}_{i} ) - {a}^{′} ( {ω}_{i} ) )}^{2} ) \qquad ( Eq . 5 )$$

In Equation 5, i represents each discrete frequency ω out of n total frequencies. The values for circuit model elements βk that minimize the error are defined as the fit values, Bₖ′. In Equation 5, the X(ω)= [symbol] (Z_{abs}(ω)) and Y(ω)= [symbol] (Z_{abs}(ω)) in which Z_{abs}(ω), shown as element 320, is defined per Equation 6.

$${Z}_{abs} ( ω ) = {R}_{solA} + {R}_{solB} + \frac{{R}_{s} ( \frac{{R}_{a}}{1 + j ω {R}_{a} {C}_{a}} + \frac{{R}_{b}}{1 + j ω {R}_{b} {C}_{b}} )}{{R}_{s} + \frac{{R}_{a}}{1 + j ω {R}_{a} {C}_{a}} + \frac{{R}_{b}}{1 + j ω {R}_{b} {C}_{b}}} \qquad ( Eq . 6 )$$

The formulation enables a single experimental apparatus, measuring circuit voltages at three points in the system across a range of frequencies, to resolve the resistances and capacitances of all the circuit elements 104a-104g shown in FIGS. 1A and 3A. Biologically, the operation allows the resolution of differences between the apical and basolateral membranes and quantification of their change in response to an external stimulus (such as drugs or ion concentration changes).

The mathematical model of Equation Set 1-5 represent a significant advancement in the field of epithelial transport electrophysiology. Unlike previous intracellular electrophysiology methods, the model can provide the measurements of the apical, basolateral, and shunt electrical transport properties without relying on assumptions, e.g., the shunt resistance is fixed or a constant percentage of TER [41′], [44′], [57′]. By eliminating the need for time-consuming and resource-intensive follow-up experiments to validate how each pathway is separately affected in an experiment, the model can significantly increase the efficiency and accuracy of drug discovery and disease research.

Further description of the model and its performance can be found in Lewallen, Colby F., et al. “A biologically validated mathematical model for decoding epithelial apical, basolateral, and paracellular electrical properties.” American Journal of Physiology-Cell Physiology 325.6 (2023): C1470-C1484, which is incorporated by reference in its entirety.

Data normalization: Normalizations can be performed during each fit, including: 1) converting the magnitude of each circuit element βₖ to a log 10 domain, and (2) normalizing the real impedance, imaginary impedance, and membrane ratio to their respective maximum values.

Because the resistors and capacitor circuit elements can have vastly different orders of magnitude, the analysis can adjust them so that the fitting algorithm assign equal or similar “weight” to the relative error of each parameter. To ensure that the magnitude of each parameter is roughly equal, the model parameters βₖ can be converted to a log-base 10 system in which \hat{β}ₖ=log₁₀(βₖ). Later, within the fitting function, the fitted parameter [symbol] can be converted back to the original, non-log-base 10 values, β′ₖ= [symbol] .

Similarly, the real impedance, imaginary impedance, and membrane ratio could also span vastly different ranges. To ensure the sum of the relative error magnitudes for each input vector D is approximately equal, the relative error magnitudes, in some embodiments, can be preprocessed by being normalized (e.g., divided or its equivalents) by the maximum magnitude such that the real impedance, imaginary impedance, and membrane ratio all spanned the range of [−1, 1]. Specifically

$$\hat{D} = \frac{D}{{max}_{d ∈ D} \vert d \vert} .$$

Then, the value for the fit real impedance, imaginary impedance, and membrane ratio magnitudes and residuals (r) can be multiplied by the maximum values that can be extracted in the preprocessing phase to return the correct magnitude. Specifically,

$${D}^{′} = \hat{{D}^{′}} {max}_{d ∈ D} \vert d \vert and r = \hat{r} {max}_{d ∈ D} \vert d \vert .$$

Fourier transform: The magnitudes of both membrane potentials as a function of frequency [|Vₐ(ω)| and |V_{b}(ω)|] can be computed from Vₐ(t) and V_{b}(t) using a short-time Fourier transform (STFT) function (e.g., stft, Matlab, Mathworks, Natick, MA or the like, e.g., Fourier Transform). In some embodiments, the Fourier transforms can be applied to small Hanning windowed segments of Vₐ(t) and V_{b}(t).

Due to abrupt changes in frequency inherent to the EIS measurement technique, there can be discontinuities in the time domain that can lead to spectral leakage when using a conventional FFT algorithm. The STFT input arguments and definitions to calculate the amplitude of Vₐ and V_{b} at each of the frequencies include parameters that can address such discontinuities, e.g., (i) signal_fraction parameter (e.g., samples in the signal that contain frequency ωₙ divided by the total number of samples in the signal); (ii) window_length parameter (e.g., 80% of the signal_fraction); and (iii) window parameter (the Hanning window function can be used to evaluate the subset of the signal that contains the frequency ωₙ, with an overlap length of 75% of the window_length).

Using an STFT operator, the amplitude |V(ω)| at each frequency can be extracted. At each frequency, the STFT can be cropped to a time region of interest (ROI). The time ROI can be defined to overcome the inherent limitations in the NOVA software by setting the time RO to begin a pre-defined period (e.g., 2 seconds) before the reported initiation time of the applied frequency by the acquisition, e.g., as controlled by an EIS software. Similarly, the time ROI can be set to end a predefined period (e.g., 2 seconds) after the start of the next applied frequency. After cropping the STFT of the time domain data, the data can then be cropped at the target frequency to generate a time and frequency ROI that contains a subset of the calculated signal amplitudes. The maximum amplitude, or stft_amp, can be extracted from the ROI. The maximum value can be converted to amplitude in which

$$\vert V ( ω ) \vert = 4 \vert \frac{stft\_amp}{window\_length} \vert .$$

In contrast to STFT, using a single window size for all frequencies can increase the measurement error because the window sizes approximately equal to the signal fraction of high frequencies can have large errors in the measured low-frequency amplitude, e.g., due to insufficient cycles inside the window. Large errors in high frequencies had been observed with larger window sizes (≅ to a signal fraction of low frequencies) that can exceed the duration of the applied high-frequency signal. Signals that start and end within a single window have reduced power in an FFT calculation and would need a correction, e.g., by dividing by the signal fraction per above. When the exact duration of any frequency is not known, the duration of each frequency can be estimated using the start time of each frequency; a value reported by the EIS software.

To improve accuracy, the analysis of a measurement can repeated for each frequency. Low frequencies (e.g., 0.5 Hz) can require a longer time to acquire, and having multiple of them (e.g., three cycles per frequency) would take even longer than at higher frequencies (e.g., 10 kHz).

Evaluation metric for fitting and parameter accuracy: To assess the efficacy of the mathematical model and fitting algorithm, error quantification can be performed, e.g., based on residual error or parameter estimation error. Residual error can gauge the ability of the model to fit the measured data at all measured frequencies (ω), e.g., using the equation, r(ω)=|d′(ω)−d(ω)| in which r(ω) is the residual error at a given frequency, and d′(ω) and d(ω) are the model-predicted and actual measurements, respectively, for either the real impedance, imaginary impedance, or membrane ratio. Parameter estimation error can be determined by the equation

$${ε}_{k} = \frac{\vert {β}_{k}^{′} - {β}_{k} \vert}{{β}_{k}} \times 100$$

in which βₖ is the estimate derived from the fitting algorithm, while βₖ is the independently measured value obtained via a multimeter. The metrics can provide a comprehensive view of model performance. Large residual errors may signal a suboptimal model choice, while significant parameter estimation errors would suggest inaccurate circuit element representation in the model.

### Example Intracellular Approximate Cell Circuit Model

FIG. 3B shows the mathematical model 322 for the minimization operation 136 of FIG. 3B (and FIG. 1B) that can be performed by the solver (e.g., 132b) to determine the approximate electrical characteristic values 104 for a cell/tissue sample. The model relies on a ratio of approximate impedances in 332 as measured by the intracellular electrode relative to the electrode in the apical or basolateral solution. The measured and fit approximated impedances are inserted into the third residual term in the objective function 324. The objective function minimization 136 provides good estimations for the parameters 104 under the condition that the nutrient solutions 116a, 116b have similar electrical characteristics.”

The minimization operation 136 has the form of Equation 13 (324) provided below. The mathematical model 110 to which Equation 9 derives is described in relation to Equations 7-12 below.

The absolute impedance Z_{abs} for an intracellular measurement can be determined by dividing the magnitude of TEP at a particular frequency by the magnitude of the applied current, i_{applied} per Equation 7.

$${Z}_{a b s} ( ω ) = \frac{\vert {V}_{1} - {V}_{2} \vert ( ω )}{\vert {I}_{1} - {I}_{2} \vert ( ω )} \qquad ( Eq . 7 )$$

In the generalized form, the value is complex, having both real and imaginary components, and can be used to separate the measured value of the complex impedance into its real and imaginary parts such that Z_{abs}(ω)=X(ω)+jY(ω), where j=\sqrt{−1} and thus X(ω) [symbol] (Z_{abs}(ω) and Y(ω)= [symbol] (Z_{abs}(ω). The representation also allows for separate measurements and analysis of the real and imaginary components of the impedance.

The magnitude of the complex impedance Z_{abs} can be mathematically modeled by combining the circuit elements in the intracellular circuit model (e.g., 110, FIG. 1B) to provide Equation 7 (above). The model for Z_{abs} is complex, and can also be rewritten per Equation set

$${Z}_{a b s} ( ω ) = P ( ω ) + j Q ( ω ) , where \qquad ( Eq . Set 8 )$$

$$P ( ω ) = {R}_{solA} + {R}_{solB} + \frac{{R}_{s} ( {R}_{a} + {R}_{b} ) ( {R}_{a} + {R}_{b} + {R}_{s} - {C}_{a} {C}_{b} {R}_{a} {R}_{b} {R}_{s} {ω}^{2} )}{{σ}_{1}} + \frac{\begin{matrix}{R}_{s} ( {C}_{a} {R}_{a} {R}_{b} ω + {C}_{b} {R}_{a} {R}_{b} ω ) \\ ( {C}_{a} {R}_{a} {R}_{b} ω + {C}_{b} {R}_{a} {R}_{b} ω + {C}_{a} {R}_{a} {R}_{s} ω + {C}_{b} {R}_{b} {R}_{s} ω )\end{matrix}}{{σ}_{1}}$$

$$Q ( ω ) = \frac{{R}_{s} ( {C}_{a} {R}_{a} {R}_{b} ω + {C}_{b} {R}_{a} {R}_{b} ω ) ( {R}_{a} + {R}_{b} + {R}_{s} - {C}_{a} {C}_{b} {R}_{a} {R}_{b} {R}_{s} {ω}^{2} )}{{σ}_{1}} - \frac{{R}_{s} ( {R}_{a} + {R}_{b} ) ( {C}_{a} {R}_{a} {R}_{b} ω + {C}_{b} {R}_{a} {R}_{b} ω + {C}_{a} {R}_{a} {R}_{s} ω + {C}_{b} {R}_{b} {R}_{s} ω )}{{σ}_{1}}$$

$$In which {σ}_{1} = {( {R}_{a} + {R}_{b} + {R}_{s} - {C}_{a} {C}_{b} {R}_{a} {R}_{b} {R}_{s} {ω}^{2} )}^{2} + {( {C}_{a} {R}_{a} {R}_{b} ω + {C}_{b} {R}_{a} {R}_{b} ω + {C}_{a} {R}_{a} {R}_{s} ω + {C}_{b} {R}_{b} {R}_{s} ω )}^{2}$$

Using the measured values of the complex impedance, the circuit parameters were calculated by minimizing the difference between the measured and modeled real and imaginary impedance at each frequency. In numerical method form, the objective function to be minimized can be expressed per Equation 9.

$${\sum }_{i = 1}^{n} ( {( X ( {ω}_{i} ) - P ( {ω}_{i} ) )}^{2} + {( Y ( {ω}_{i} ) - Q ( {ω}_{i} ) )}^{2} ) \qquad ( Eq . 9 )$$

In Equation 9, i represents each discrete frequency, ω, out of n total measurements. However, there can be an infinite number of values Rₐ, R_{b}, Cₐ, C_{b}, Rₛ, R_{solA}, R_{solB} for which the objective function in Equation 9 can be equally minimized. Thus, the system of equations is under-constrained.

It was observed in the study described herein that only one additional, independent measurement of the circuit in the intracellular circuit model (e.g., 110) could be acquired, to which the mathematical model can be fully defined for the system. The study described herein experimentally validated the approach. The study employed the impedance ratio (Zᵣ), expanding on the traditional VDR in [2] by recording the VDR at many frequencies.

Using multiple measurements and different frequencies, the impedance ratio (Zᵣ), shown as element 332, can be defined as the ratio of the complex impedance of the apical and basolateral membranes (Zₐ and Z_{b}, respectively) per Equation 10.

$${Z}_{r} ( ω ) = \frac{\vert {Z}_{a} ( ω ) \vert}{\vert {Z}_{b} ( ω ) \vert} = \frac{\vert {V}_{a} ( ω ) \vert}{\vert {V}_{b} ( ω ) \vert} \qquad ( Eq . 10 )$$

In Equation 10, a Short Time Fourier Transform (STFT) can be used to determine |Vₐ(ω)| and |V_{b}(ω)|. Zᵣ(ω) is a magnitude and is thus a real number.

Similarly, the mathematical model of the impedance ratio (R(ω)) can be derived as a function of the membrane parameters in the intracellular circuit model (e.g., 110) per Equation Set

$$R ( ω ) = \frac{\vert {Z}_{a} ( ω ) \vert}{\vert {Z}_{b} ( ω ) \vert} , where \vert {Z}_{a} ( ω ) \vert = \sqrt{\frac{{C}_{a}^{2} {R}_{a}^{2} {R}_{s o l A}^{2} {ω}^{2} + {R}_{a}^{2} + 2 {R}_{a} {R}_{s o l A} + {R}_{s o l A}^{2}}{{C}_{a}^{2} {R}_{a}^{2} {ω}^{2} + 1}} \vert {Z}_{b} ( ω ) \vert = \sqrt{\frac{{C}_{b}^{2} {R}_{b}^{2} {R}_{s o l B}^{2} {ω}^{2} + {R}_{b}^{2} + 2 {R}_{b} {R}_{s o l B} + {R}_{s o l B}^{2}}{{C}_{b}^{2} {R}_{b}^{2} {ω}^{2} + 1}} . \qquad ( Eq . Set 11 )$$

Because the membrane ratio is a ratio of magnitudes, it is not a complex number and has no units. The mathematical model of the membrane ratio (R′(ω)) can be derived as a function of the circuit parameters in FIG. 1B per Equation 12.

$${R}^{′} ( {ω}_{i} ) = \frac{{Z}_{a} ( ω ) {R}_{s} + {R}_{s o l A} ( {Z}_{a} ( ω ) + {Z}_{b} ( ω ) + {R}_{s} )}{{Z}_{b} ( ω ) {R}_{s} + {R}_{s o l B} ( {Z}_{a} ( ω ) + {Z}_{b} ( ω ) + {R}_{s} )} \qquad ( Eq . 12 )$$

In Equation 12, when ω is very low (e.g., 0.5 Hz), and when R_{solA} and R_{solB} are both much lower than Rₐ and R_{b}, that Zᵣ≅VDR. Each circuit element in FIGS. 1B an 3B can be treated as an element in an array, βk, where the subscript k represents the kth element in the array (e.g., β₁=R_{solA}, β₂=Rₐ, etc.). The measured values for the real impedance Zₐ(ω), the imaginary impedance Z_{b}(ω), and the membrane ratio Zᵣ(ω) can be modeled by a unique combination of βₖ to calculate |Zₐ(ω)|, |Z_{b}(ω)| and R′(ωᵢ) per Equation 10, Equation Set 11, and Equation 12.

The membrane parameters Rₐ, Cₐ, R_{b}, C_{b}, Rₛ, R_{solA}, R_{solB} can be calculated by minimizing the error between each measured and modeled value for the complex impedance and the impedance ratio. In numerical method form, the objective function to minimize can be expressed as Equation 13 (shown in FIGS. 1B and 3B).

$${\sum }_{i = 1}^{n} ( {( X ( {ω}_{i} ) - P ( {ω}_{i} ) )}^{2} + {( Y ( {ω}_{i} ) - Q ( {ω}_{i} ) )}^{2} + {( {Z}_{r} ( {ω}_{i} ) - R ( {ω}_{i} ) )}^{2} ) \qquad ( Eq . 13 )$$

The system can be solved using a nonlinear fitting algorithm, iteratively to minimize the difference, or error, between the measured and modeled values to obtain a unique set of membrane-specific properties of FIG. 1B. These resulting values that minimize the error are defined as the fit values. The formulation enabled a single experimental apparatus, measuring circuit voltages at three points in the system across a range of frequencies, to resolve the resistances and capacitances of all the circuit elements shown in Error! Reference source not found. when the nutrient baths have similar characteristics or to provide approximate resistances and capacitances of the cellular components. Biologically, the operation allows the resolution of differences between the apical and basolateral membranes when the nutrient baths have similar characteristics and their change (apical and basolateral membranes) in response to external stimuli (such as drugs or ion concentration changes).

Additional background and derivation information can be found in Lewallen, Colby, Epithelial Electrophysiology Using Intracellular Robotics and Extracellular Impedance Spectroscopy (thesis), 2022, http://hdl.handle.net/1853/66173, which is incorporated by reference in its entirety.

### Time-Constant Impedance Ratio Analysis Circuit Model

FIG. 3C shows an example of an extracellular electric model, as an impedance model, to which approximate or impedance elements of the circuit models can be determined from a fitting operation that can then be used to calculate the time-constant impedance ratio (tau ratio). The circuit model includes a mathematical model of RPE comprising two parallel RC filters, in series with each other. Other equivalent circuit models can be similarly applied, e.g., having additional multiple parallel RC circuits, though they could be reduced to the model shown in FIG. 3C. The time-constant impedance ratio (tau-ratio) can be calculated from the magnitude of the values in the model after fitting. Specifically, time-constant impedance ratio (tau-ratio)=R₁*C₁/(R₂*C₂) or R₂*C₂/(R₁*C₁).

The parameters, R₁, C₁, R₂, C₂ can be determined from a minimization operation of an objective function J, where J is defined per Equation Set #14.

$$J = {\sum }_{i = 1}^{n} ( {( X ( ω ) - {X}^{′} ( ω ) )}^{2} + {( Y ( ω ) - {Y}^{′} ( ω ) )}^{2} ) , where {X}^{′} = ℝ ( {Z}_{12} ) {Y}^{′} = 𝕀 ( {Z}_{12} ) X = Measured real {Z}_{12} Y = Measured real {Z}_{12} \qquad ( Eq . Set 14 )$$

In Equation Set #14, [symbol] is a real number, [symbol] is the imaginary values, ω is the measurement frequency in rad/s, and the total impedance Z₁₂(ω) of the extracellular circuit model is defined per Equation 15 where j=\sqrt{−1}.

$${Z}_{12} ( ω ) = {R}_{s o l} + \frac{{R}_{1}}{1 + j ω {R}_{1} {C}_{1}} + \frac{{R}_{2}}{1 + j ω {R}_{2} {C}_{2}} , where \qquad ( Eq . 15 )$$

Following the fitting operation that provides the parameters R₁, C₁, R₂, C₂, the time-constant impedance ratio (tau-ratio) can be calculated from the magnitude of the values per tau-ratio=R₁*C₁/(R₂*C₂) or R₂*C₂/(R₁*C₁). The tau ratio value can have a range of [0 . . . 1] or [1 . . . ∞]. The derivation, and analysis, of the time-constant impedance ratio (tau ratio) has similarities to the impedance ratio objective function as described in relation FIGS. 3A and 3B. First, they both require the defining of a net impedance term Z as a function of frequency ω. The impedance Z is mathematically complex, and thus, contains the sum of a real component and an imaginary component (i.e., sqrt(−1)). Additionally, they both rely on minimizing an objective function J in which the parameters making up the real and imaginary portions of the complex impedance Z are fitted by the algorithm such that they minimize the sum of all the residuals between the measured and modeled values.

In contrast, the analysis of the time-constant impedance ratio (tau ratio) has meaningful distinctions to the impedance ratio objective function as described in relation FIGS. 3A and 3B. The tau ratio analysis involves minimizing two terms in the objective function and applies to the circuit shown in FIG. 1C, namely, the real impedance of Z₁₂ and the imaginary impedance of Z₁₂. The impedance ratio, in contrast, involves minimizing three terms in the objective function and applies to the circuit shown in FIGS. 1A and 1B, namely, the real impedance of Z_{abs}, the imaginary impedance of Z_{abs}, and the impedance ratio. Z_{abs} and Z₁₂ represent the complex impedance of different circuits. Z₁₂ is a simplified model of Z_{abs}, essentially merging the paracellular (or shunt) resistance into Rₐ, Cₐ, R_{b}, and C_{b}.

### Example EIS Measurement Device and Instrument

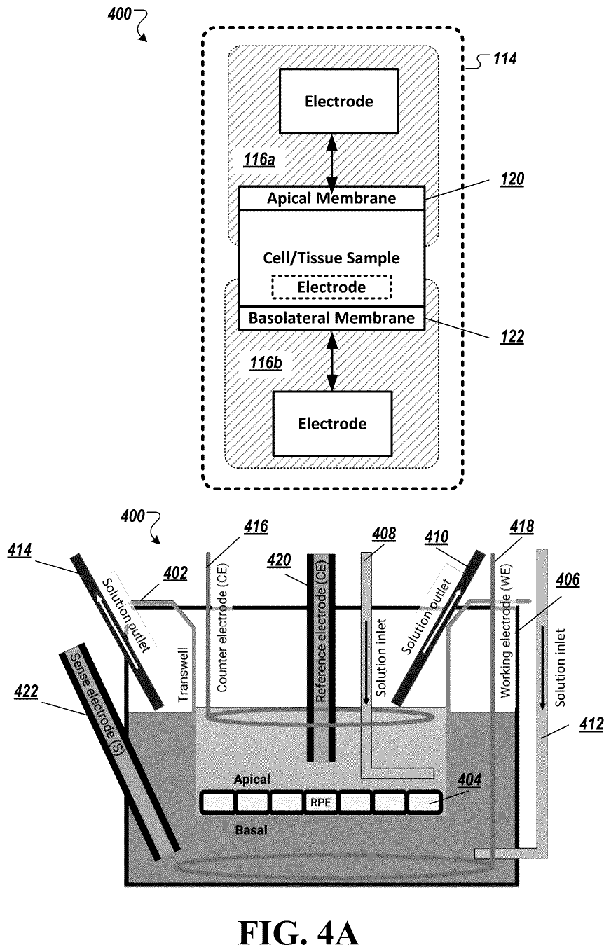

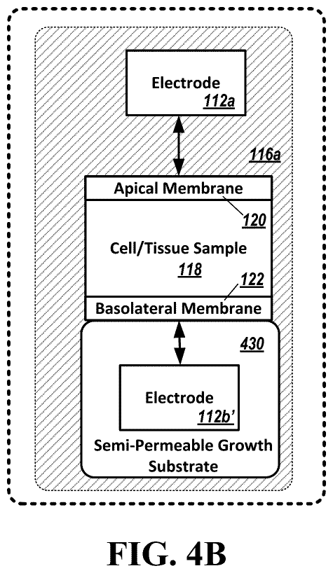

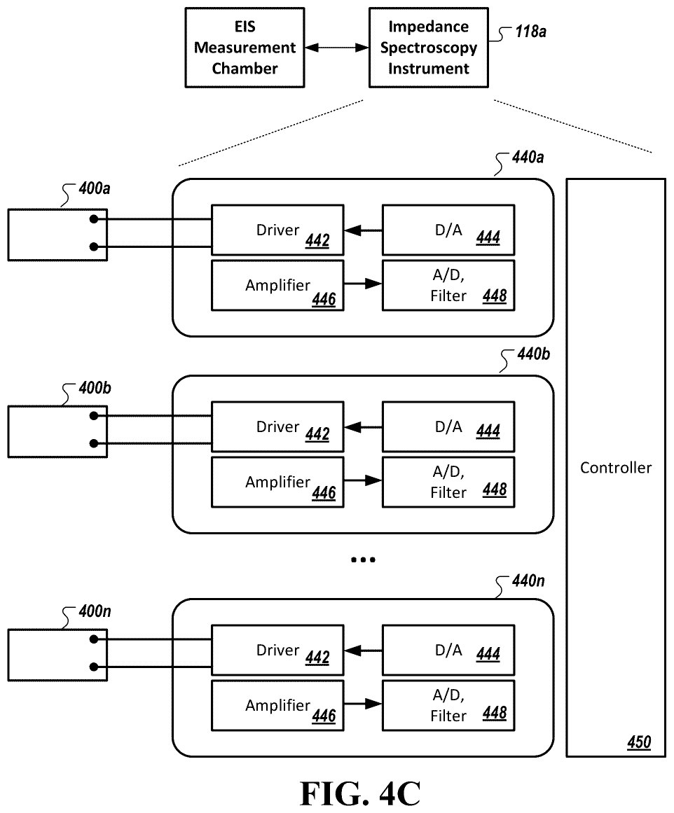

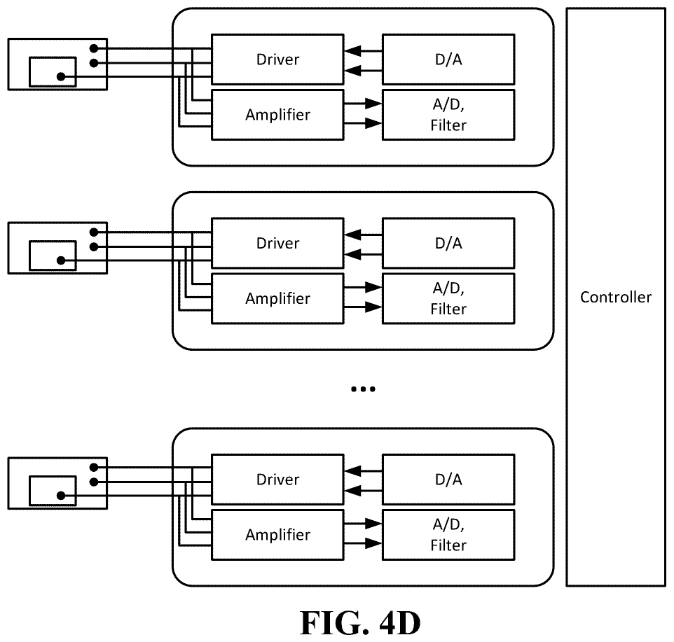

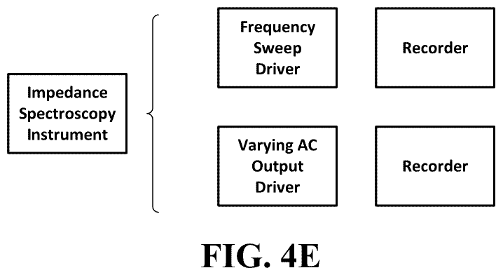

Example Measurement Device. FIG. 4A depicts an exemplary measurement device 400 comprising chamber 114. The measurement chamber device can be employed in any one of the exemplary systems (e.g., 100a, 100b, or 100c), though other measurement chambers can be used, including those described or referenced herein. The cell/tissue sample 118 has an apical membrane 120 and a basolateral membrane 122; the cell/tissue sample 118 is cultured on a semi-permeable growth substrate. In some embodiments, the semi-permeable growth substrate is Corning 3460, Greiner 665641, SPL2 37012, VWR 76313-904, or Nest 724121. In some aspects, the semi-permeable growth substrate comprises polylactic acid, polyglycolic acid, polylactic-co-glycolic acid, or any combination thereof.

The apical membrane 120 of the cell/tissue sample 118 is in contact with a first nutrient solution bath 116a, and the basolateral membrane 122 is in contact with a second nutrient solution bath 116b. In some embodiments, the first nutrient solution bath 116a and the second nutrient solution bath 116b are the same. In some embodiments, the first nutrient solution bath 116a and the second nutrient solution bath 116b are different. In some embodiments, the first nutrient solution bath 116a and/or the second nutrient solution bath 116b can include ionic salts, amino acids, sugars, or any combination thereof. In some embodiments, the ionic salts, amino acids, or sugars, can include KCl, NaCl, NaHCO₃, MgCl₂, CaCl₂, taurine, glucose, sucrose, or any combination thereof. In some embodiments, both the first nutrient solution bath 116a and the second nutrient solution bath 116b include Ringer solution.

A first electrode 112a is operably coupled to the first nutrient solution bath 116a, and a second electrode 112b is operably coupled to the second nutrient solution bath 116b. In some embodiments, the first electrode 112a and/or the second electrode 112b are operably coupled to the first nutrient solution bath 116a and the second nutrient solution bath 116b, respectively, via salt bridges. A third electrode 112c can be operably coupled to an intracellular space between the apical membrane 120 and the basolateral membrane 122. In some embodiments, the measurement chamber 114 does not include a third electrode 112c. In some embodiments, the third electrode 112c is operably coupled to a different part of the cell/tissue sample.

Epithelial tissues line our organs and form critical barriers in our body. Damage and degeneration of these tissues are associated with numerous common diseases, such as celiac disease [1], cystic fibrosis [2], diabetes [3], and age-related macular degeneration [4]. Epithelial cells, which make up these tissues, form tight connections with their neighbors and form a selective barrier to ions, nutrients, and waste products [5],[6]. As a result, these barrier-type tissues play a critical role in maintaining organ homeostasis and function [5],[7].

FIG. 4A also shows an example of the measurement chamber 114 (shown as 400). The measurement chamber 400 includes a Transwell 402 configured to hold an epithelial cell/tissue sample 118 (shown as “RPE” 404). The epithelial cell/tissue sample 404 includes an apical side that faces the inside of the Transwell 402 and a basal side that faces away from the Transwell 402. The Transwell 402 is placed within a second container 406. The Transwell 402 includes an inlet 408 (shown as “Solution inlet” 408) that extends into the Transwells 402 into its lower portion (e.g., the bottom of Transwell) and an outlet 410 (shown as “Solution outlet” 410) that extends into the Transwell 402 into its upper portion (e.g., top of the nutrient bath).

The second container 406 includes an inlet 412 (shown as “Solution inlet” 412) that extends into the container 406 into its lower portion (bottom of the container) and an outlet 414 (shown as “Solution outlet” 414) that extends into the container 406 into its upper portion (e.g., top of nutrient bath).

The Transwell 402 includes a first electrode (e.g., 112a) comprising a counter electrode 416, and the second container 406 includes a second electrode (e.g., 112b) comprising a working electrode 418. In some embodiments, the working electrode 418 and counter electrode 416 is employed to provide a current for the measurement, e.g., from the working electrode 418 to the counter electrode 416. The measurement chamber 400 also includes a reference electrode 420 and a sense electrode 422 to sense the measurement. In some embodiments, the reference electrode is connected to a basal side of the tissue, and the sense electrode is connected to the apical side of the tissue through an agar bridge, e.g., to provide a voltage measurement. Other configurations can be used as described or referenced herein.

Non-semipermeable growth substrate. FIG. 4B shows an example EIS system 100 (shown as 100d) employing a solid non-semipermeable growth substrate that can be used, e.g., for the system of FIGS. 1a-1C. Rather than a bath 116a, 116b, an embedded electrode, shown as 112b′, though it can be employed for either 112a or 112b, is embedded in a single solid non-semipermeable growth substrate 430 to which the cell/tissue sample 112 is cultured or placed thereon. The embedded electrode (112a′ or 112b′) is embedded in the non-semipermeable growth substrate 430, which is then placed in the solution (not two), and one of the other electrodes would be in direct contact with the cell/tissue.

In the example, the Rₛₒm model 110 now represents the combined non-semipermeable growth substrate and bath solution and can still be determined using processes described in relation to FIGS. 1A, 1B, 1C. In FIG. 1D, the third electrode 112c is shown as optional.

### Example Impedance Spectroscopy Instrument

FIGS. 4C and 4D each show an example of an impedance spectroscopy instrument (e.g., 118). In FIG. 4C, the impedance spectroscopy instrument 118 (shown as 118a) is configured as a massively parallel monitoring system that includes circuitries for multiple channels 440 (shown as 440a, 440b, . . . 400n) to provide extracellular EIS measurements for a set of measurement chambers (e.g., 118, 400) shown as 400a, 400b, . . . , 400n. Each channel (440a-440n) includes a driver circuit 442, D/A circuit 444, amplifier circuit 446, and A/D circuit and filter 448. The D/A circuit 444 and A/D circuit 448 are coupled to a controller 450 configured to interrogate the samples in the measurement chambers 400a-400n according to a pre-defined user setting (e.g., 1-minute increment, 15-increment, 30-minute increment, 60-minute increment, 2-hour increment, 3-hour increment, 4-hour increment, daily, etc.), e.g., in a method described in relation to FIGS. 2F, 2G.

In the example shown in FIG. 4D, the impedance spectroscopy instrument 118 (shown as 118b) is configured to provide three electrode measurements in the measurement chamber.

FIG. 4E shows configurations of the impedance spectroscopy instrument 118. The instrument (e.g., 118, 118a, 118b) can include a frequency sweep driver configured to generate an oscillatory current or voltage output at different frequencies, starting at a first frequency and incrementing the frequency of the output in a pre-defined increment to an end frequency. The sweep can be over a portion of the frequency ranges of interest for the cell, which can be determined readily for a given cell type. For epithelial cells, for example, RPE, the sweep can be over a portion of the frequencies between 0.5 Hz and 1 MHz, e.g., 2 Hz to 10 Hkz, 50 Hz to 20 kHz, 100 Hz to 100 kHz, 1 kHz to 1 MHz, among other ranges. The increments of the sweep can be logarithmic scale and applied at different base frequencies, e.g., 20 Hz->200 Hz->2 kHz->20 kHz, etc. The larger the swept frequencies, the higher the accuracy can be determined up to an asymptotic threshold to which additional frequency measurements or ranges would not yield additional improvements. To improve the acquisition speed, the swept frequencies can be narrowed to narrower spans of frequencies sensitive for the epithelial cells or can be applied with less repetition.

In some embodiments, the sweep can include two or more waveforms phased shifted from one another. The interrogation may include a current waveform having less than 10 μA in amplitude, e.g., 1 μA (peak-peak), 2 μA (peak-peak), 3 μA (peak-peak), 4 μA (peak-peak), 5 μA (peak-peak), 6 μA (peak-peak), 7 μA (peak-peak), 8 μA (peak-peak), 9 μA (peak-peak), 10 μA (peak-peak). In some embodiments, the current is user-defined up to 20 μA (peak-peak). In some embodiments, the current is user-defined up to 1 mA (peak-peak).

In another configuration, the instrument (e.g., 118, 118a, 118b) can include a varying AC output driver configured to generate pre-defined oscillatory current or voltage output waveforms at pre-defined frequencies. The frequency sweep driver can provide a comprehensive measurement, while the later can provide quicker testing.

### EXPERIMENTAL RESULTS AND ADDITIONAL EXAMPLES

### Example #1—Biologically Validated Mathematical Model for Decoding Epithelial Apical, Basolateral, and Paracellular Electrical Properties

A study was conducted to develop and evaluate a technique to evaluate membrane-specific properties of epithelia by combining intracellular and extracellular electrophysiology while measuring at many frequencies to perform electrochemical impedance spectroscopy and fitting to a mathematical model with additional constraints. The study was able to determine the apical, basolateral, and shunt properties, Rₐ, R_{b}, Cₐ, C_{b}, and Rₛ. The study experimentally tested the exemplary method using a model electrical circuit with known resistances and capacitances to validate the fitting algorithm within the biological range.

The study subjected patient-derived iPSC-derived RPE to ATP to observe changes in cell and membrane electrical properties. When compared to conventional extracellular electrophysiology measurements of TER, and intracellular measurements of Rₐ/R_{b}, extracellular or intracellular measurements alone can show expected changes during ATP administration but can overlook membrane-specific values which have revealed unexpected, and perhaps undesirable, changes in response to stimuli as seen with Rₐ. The study observed that assumptions that rely on Rₛ being constant does not appear valid as assumed in previous papers.

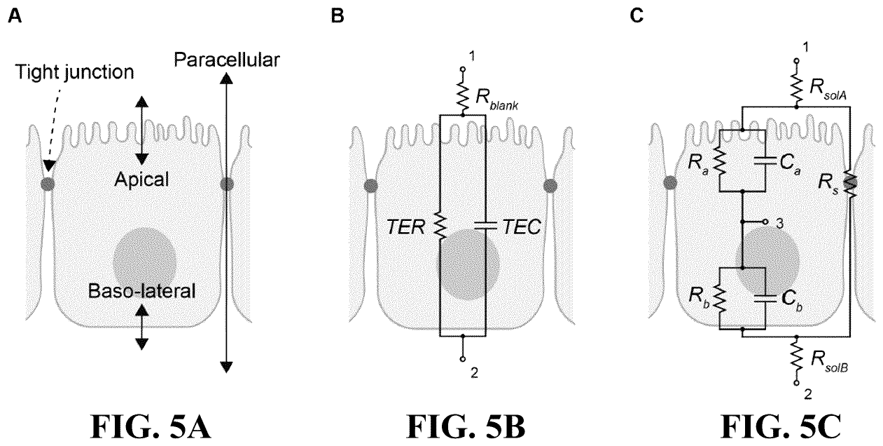

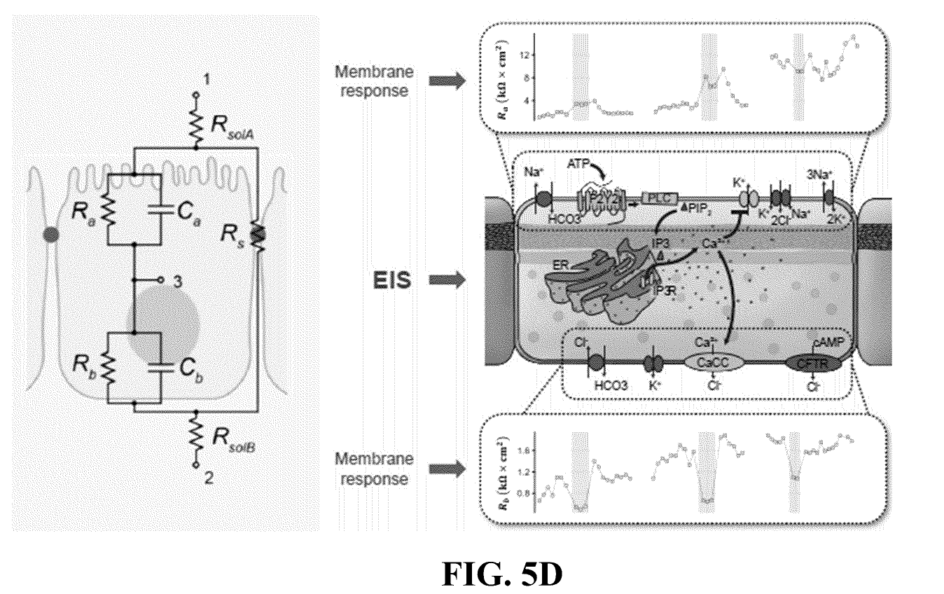

FIGS. 5A-5C show models of ion transport in epithelia employed in a study conducted to develop and validate a mathematical model for decoding epithelial apical, basolateral, and paracellular electrical properties. FIG. 5A shows a physical model of a cross-section schematic of an epithelial tissue showing the three pathways where transport is regulated. FIG. 5B shows an extracellular circuit model having a resistor-capacitor (RC) circuit that can measure trans-epithelial resistance (TER) and trans-epithelial capacitance (TEC). While widely used in the field, it has low fidelity and can be measured with commercial instrumentation. FIG. 5C shows an intracellular circuit model of ion transport that considers separate electrical properties of the apical, basolateral, and shunt pathways: Rₛ shunt pathway resistance; Rₐ and Cₐ apical resistance and capacitance, respectively; R_{b} and C_{b} basolateral resistance and capacitance, respectively [25].

FIG. 5D shows the exemplary method of the study being used to determine the electrical transport parameters of intact epithelial tissues. Indeed, the exemplary method can decipher unique epithelial tissue electrical properties, offering a benchmark for drug discovery and cell therapy quality control. In FIG. 5D, an example mechanism of RPE response is shown to apical ATP (highlighted) considered in the study. ATP appears to stimulate purinergic receptor P2Y2 on the apical membrane, which triggers a release of PIP2 that, in turn, results in an increase in cytosolic IP3. In response to this change, the endoplasmic reticulum (ER) releases calcium that has opposite electrical effects on the apical and basolateral membranes. The apical membrane has been shown to contain calcium-deactivated potassium channels that increase Rₐ, and the basolateral membrane has been shown to contain calcium-activated chloride channels (CaCC) that decrease the R_{b}.

The term ‘basolateral’ in FIGS. 5A-5C refers to the apparent (i.e., measured) impedance of the combination of the true basal and lateral cell membrane impedances. Furthermore, the “shunt” resistance encapsulates paracellular transport pathway resistances and any experimental artifacts like electrical current leak through the boundary between the Ussing chamber and the epithelial cell layer. Paracellular pathways in epithelial cells usually involve both tight junctions and the lateral intercellular space (LIS), connected in series. Without inserting an additional intermediate pipette into the LIS, by using multiple intracellular pipettes, or by making assumptions about membrane-properties, it is not possible to distinguish between the resistances of the tight junction and the LIS. Therefore, FIG. 5C lumps all non-transcellular transport pathways into a single ‘shunt’ resistor. For an in-depth derivation of the circuit model in FIG. 1C, consult references [41], [42].

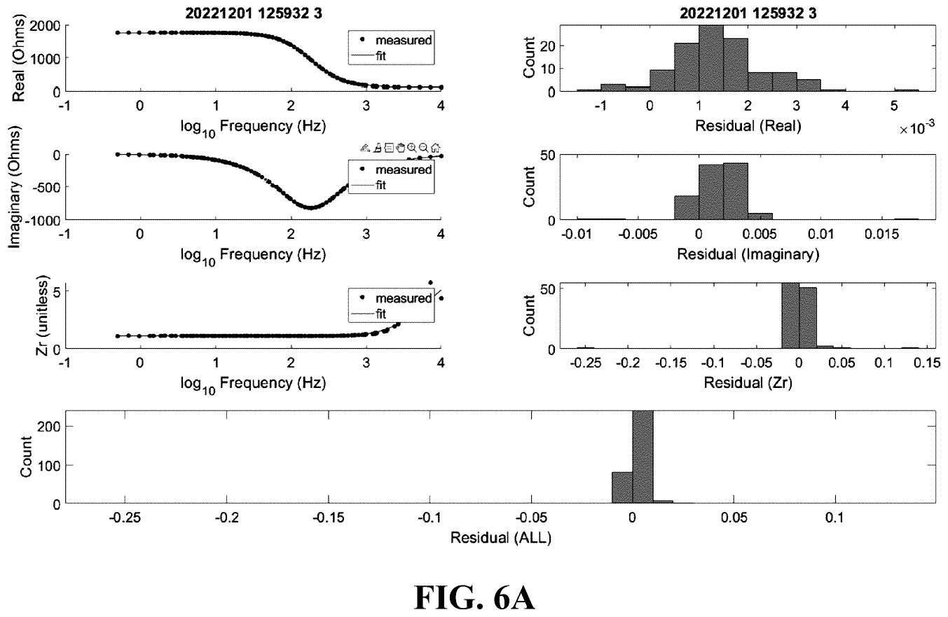

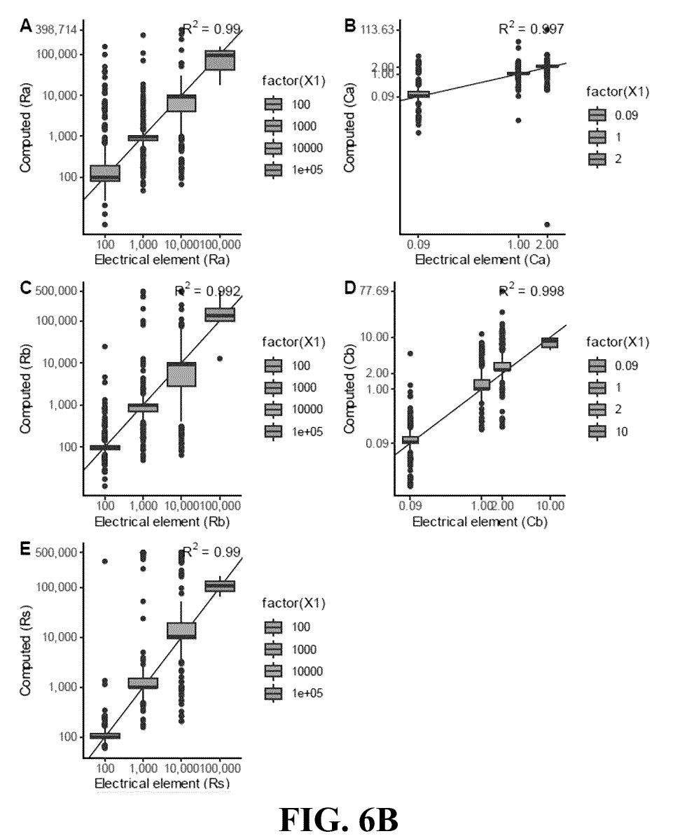

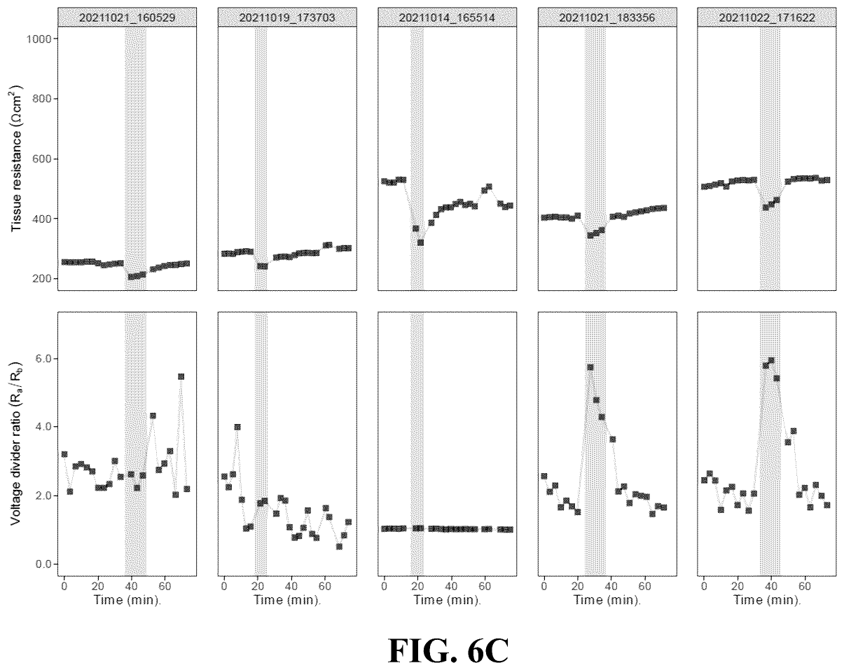

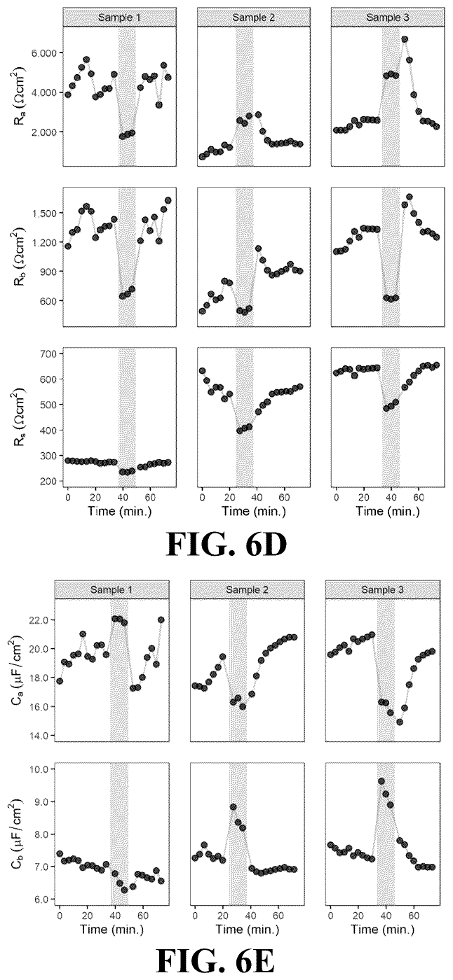

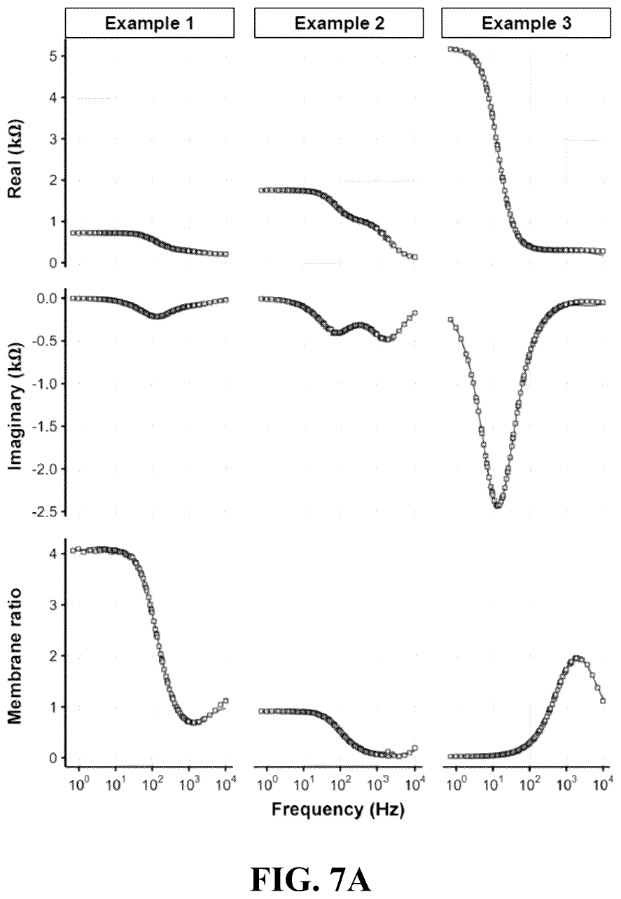

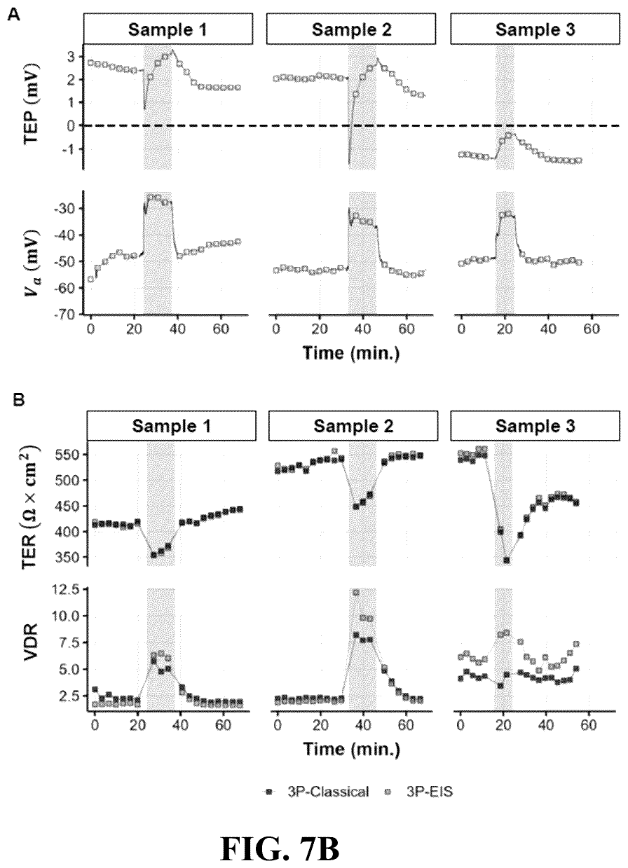

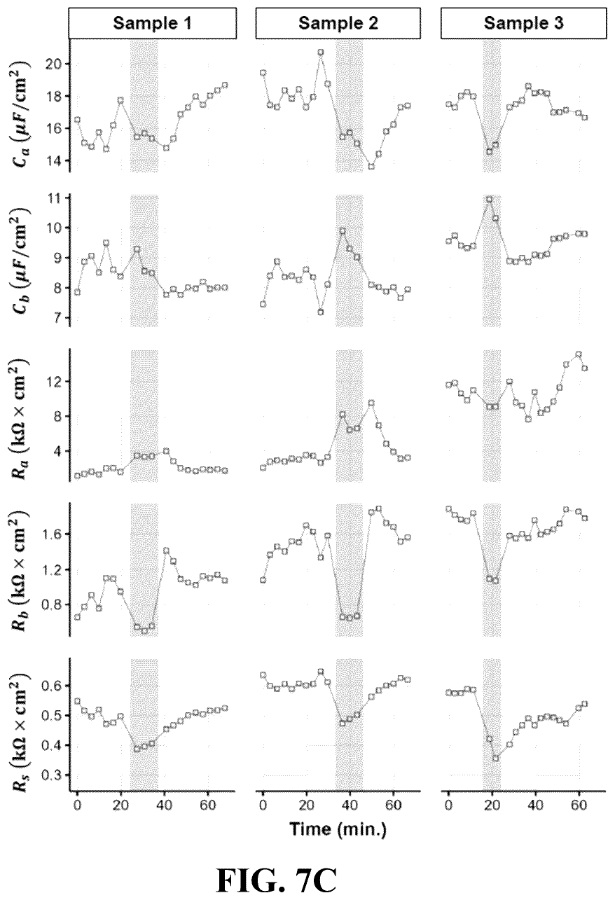

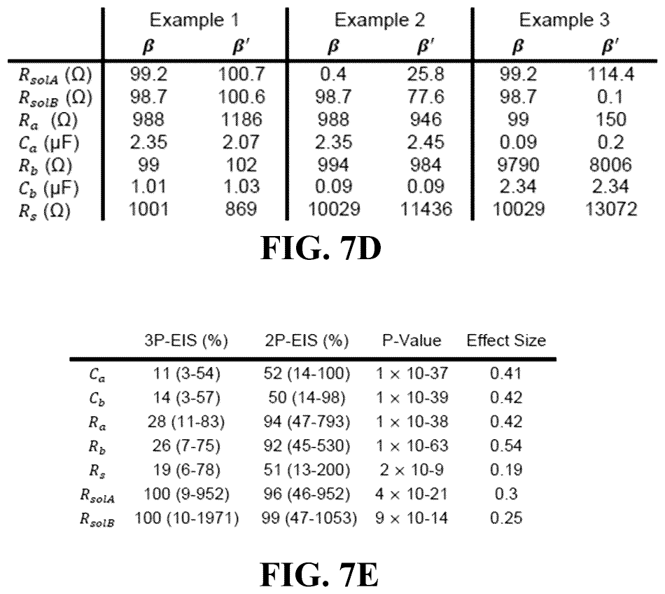

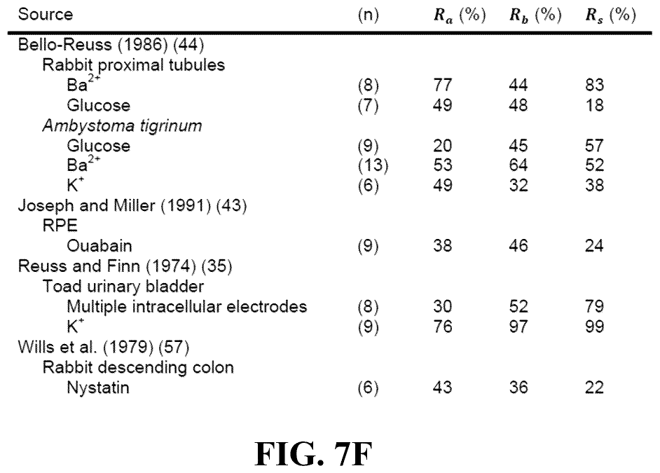

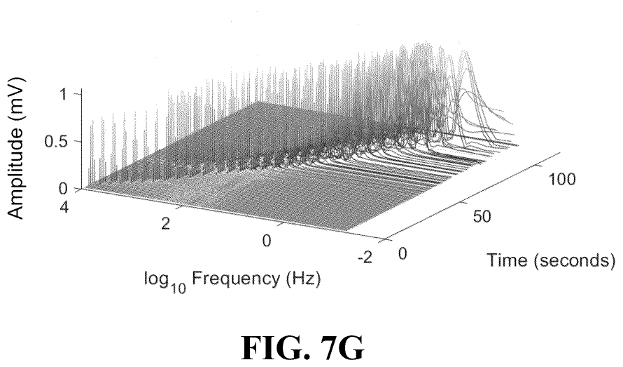

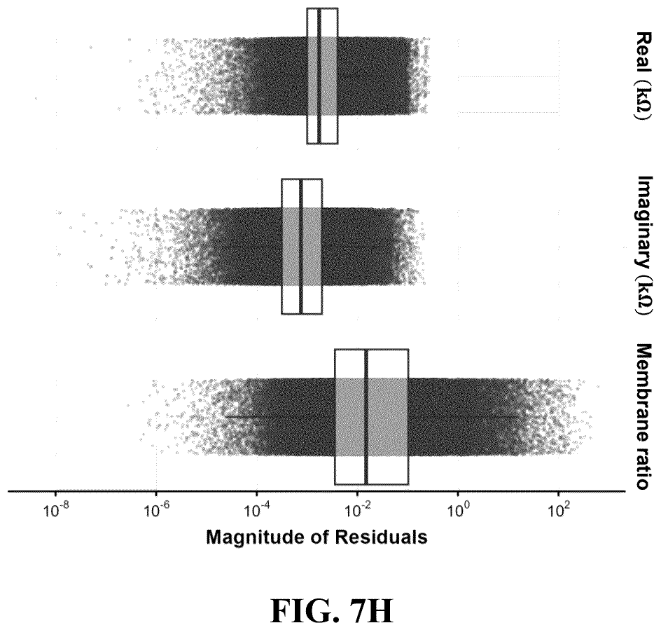

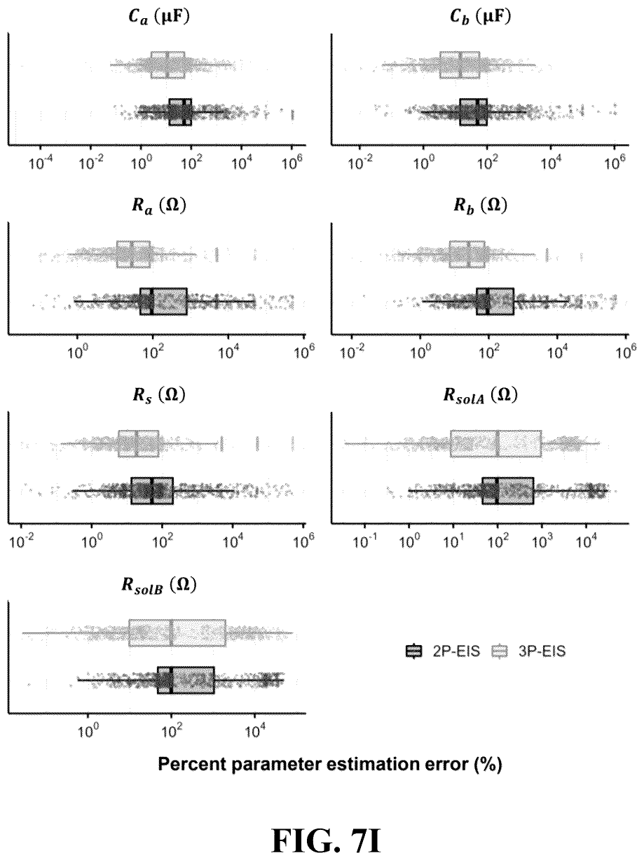

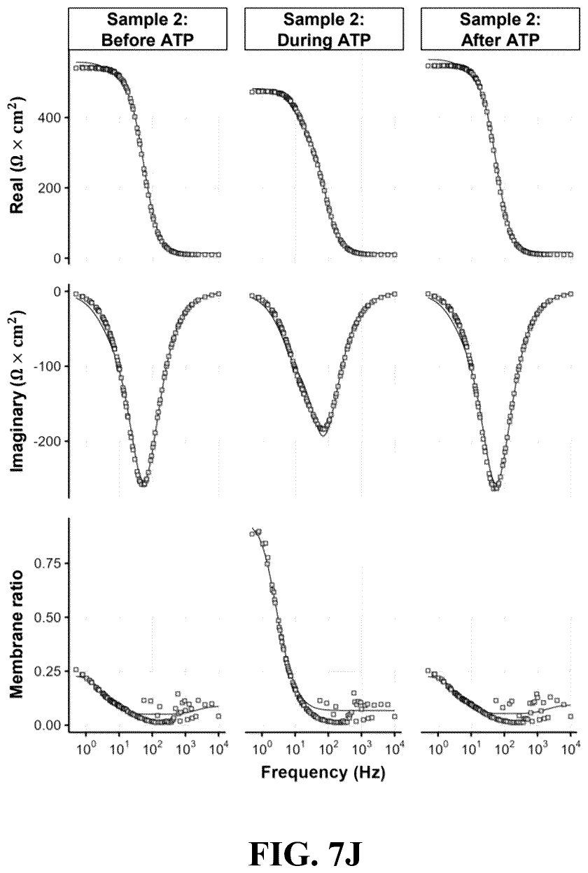

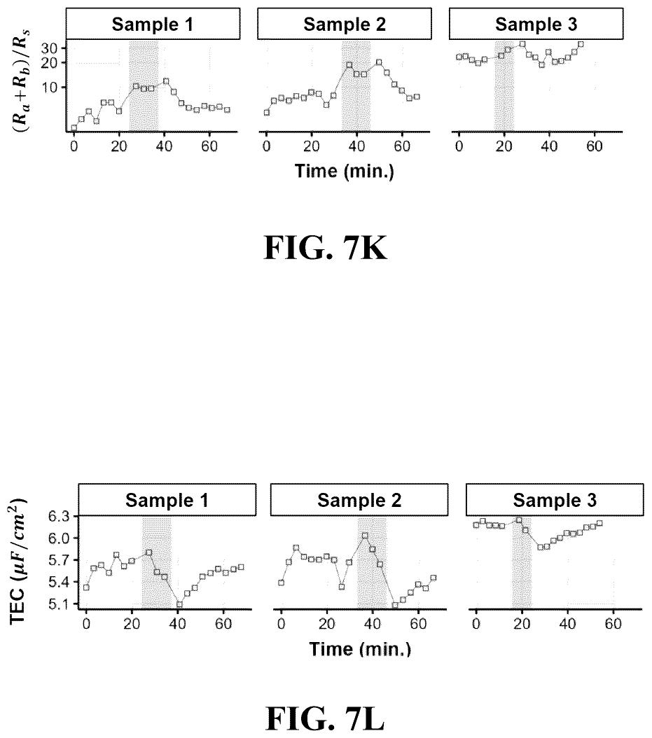

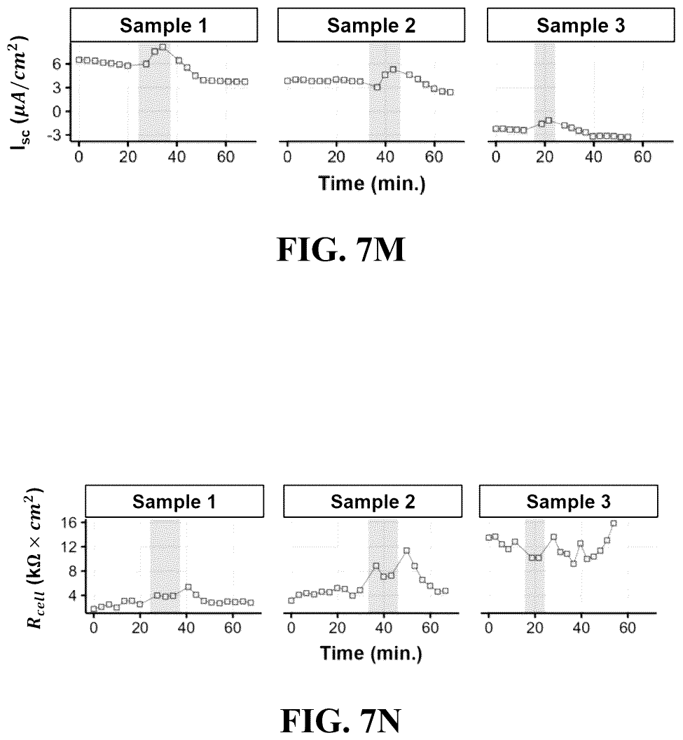

FIGS. 6A-6E show additional experimental results conducted in the study to evaluate the exemplary method. FIGS. 7A-7F show additional experimental results of experimental results in comparison to a baseline analysis.

Additional experimental results and discussion from the study can be found in Lewallen, Colby F., et al. “A biologically validated mathematical model for decoding epithelial apical, basolateral, and paracellular electrical properties.” American Journal of Physiology-Cell Physiology 325.6 (2023): C1470-C1484, which is incorporated by reference herein.

Study Background. Epithelial tissues line the organs and form critical barriers in our body. Damage and degeneration of these tissues are associated with numerous common diseases such as celiac disease [1], cystic fibrosis [2], diabetes [3], and age-related macular degeneration [4]. Epithelial cells, which make up these tissues, form tight connections with their neighbors and form a selective barrier to ions, nutrients, and waste products [5],[6]. As a result, these barrier-type tissues play a critical role in maintaining organ homeostasis and function [5], [7]. The function of such cells can be characterized through morphology [8], protein biomarkers [9], and gene expression [10], but electrophysiology [4] is one of the few techniques that beneficially allows the characterization of the cell processes in real-time, as it does not require the cells to be suspended, fixed, or lysed. This is advantageous because it provides insight on cellular transport in an environment similar to in vivo conditions—characterizing specifically how the cells regulate the transport of critical ions, nutrients, and waste products in a monolayer [4], [11].

The most common measurement of epithelial electrophysiology is trans-epithelial resistance (TER), which is used to assess the formation of tight junctions [4], [12]-[21]. TER, as shown in FIG. 5B, is readily measurable using set(s) of electrodes on either side of the tissue layer with commercially available devices (e.g., Ussing chamber (Physiologic Instruments; AD Instruments), EndOhm or STX2 (World Precision Instruments)) [22]. However, to model the epithelial monolayer as a simple resistor, or even a parallel resistor and capacitor circuit, is an oversimplification of ion transport, as there are three distinct points across the cells at which transport is regulated: the apical membrane, the basolateral membrane, and the tight junctions, as shown in FIG. 5A [23],[24]. Each of the membranes can have different permeabilities to ions. Therefore, to characterize cell transport and determine specific ion channel dysfunctions, the model would have to accurately characterize each transport pathway separately, as shown in FIG. 5C.

The intracellular circuit model of FIG. 5C, discussed in relation to FIG. 1A, can not be determined using commercially available devices that attempt to resolve the magnitude of membrane-specific parameters Rₛ, Rₐ, Cₐ, R_{b} and C_{b} only measuring extracellular measurement locations. Extracellular measurements (at nodes 1 and 2 per Error! Reference source not found.B and Error! Reference source not found.C) alone cannot differentiate the contributions of the transcellular versus paracellular pathways; thus, the system is underdefined. To resolve this issue, some have made assumptions about the circuit parameters to reduce the number of unknowns, such as assuming Rₛ to be constant, practically infinite, or a constant percentage of the total TER. The assumptions in experiments can lead to misleading results due to the over-simplification [21], [25]-[27].

It has been further observed that the assumptions about Rₛ do not hold true during the monolayer's response to a stimulus. The exemplary method was developed, building upon work that had shown the solution of the five parameters for Necturus Gallbladder epithelia [28]. A study was conducted using additional mathematical constraints, experimental electrical validation, and a biological model using retinal pigment epithelia (RPE). The study advanced state of the art, showing it is possible to solve for all intracellular circuit parameters in Error! Reference source not found.C by combining traditional intracellular electrophysiology with electrochemical impedance spectroscopy (EIS) to reveal membrane-specific properties. The exemplary method can reduce the need for additional follow-on experiments with channel blockers to further distinguish which pathways are responsible for tissue changes. Therefore, the exemplary method can provide a straightforward method to determine membrane-specific epithelial transport function, e.g., for the evaluation of epithelial diseases and validation of cellular therapies.

Measuring these individual properties has the potential to enable better research and understanding of transport changes. This ability to measure individual membrane properties can be used to study epithelial transport dysfunction and drug responses in disease models as a quality control metric for epithelial cell therapies such as retinal pigment epithelia implants for treating age-related macular degeneration.

Human iPSC-RPE cells. The study performed the protocol for induced pluripotent stem cell (iPSC) differentiation described in [29]. Briefly, for each individual iPSC line, somatic cells (blood or fibroblasts) were isolated from a single donor and were reprogrammed using the Sendai virus or episomal reprograming methods [44]. Reprogramed iPSC colonies were differentiated into RPE using the protocol described by Sharma et al. [30]. iPSC-derived RPE cells were seeded onto Corning 3406 Transwells, with a seeding density of 300,000 cells/cm² and 250,000 cells/cm² and were measured at approximately 300 days old. Fully-mature RPE cells were characterized with a transmission electron microscope (TEM) for morphology, gene expression, and immunostaining for RPE-specific markers. For iPSC genotype, TaqMan genotyping assays (ThermoFisher) were used. The following probes were tested: rs1061170 (Assay ID: AHI1TPW; 4331349); rs2230199 (Assay ID: C_26330755_10; 4351379). SNP rs10490924 (Assay ID: C_29934973_20; 4351379). Two human donors were used to derive iPSC-RPE for this experiment. Samples 1 and 2 were derived from donor A, and sample 3 was derived from donor B.

For differentiation, iPSCs were seeded onto vitronectin (A1700, ThermoFisher)-coated six-well plates. After 2 days in E8 media (A1517001, ThermoFisher) supplemented with Rock Inhibitor (1254, Tocris Bioscience), iPSC colonies formed a confluent monolayer, and were transitioned to the differentiation media ((DMEM/F12 (11330032, ThermoFisher), N2 supplement (A1370701, ThermoFisher), B27 (17504044, ThermoFisher), KSR (12618013, ThermoFisher), 20 ng/ml NOGGIN (6057 R&D Systems), 5 μM CK1-7 Dihydrochloride (C0742, Sigma), 5 μM SB 431542 hydrate (S4317, Sigma), and 5 ng/ml IGF-1 (AFL291, R&D Systems), 5 μM PD0325901 (PZ0612, Sigma), 10 mM nicotinamide (N0636, Sigma), 150 ng/ml ACTIVIN A (338-AC/CF, R&D Systems)). Cells that reached the RPE committed phase of differentiation were reseeded onto fresh vitronectin-coated surfaces and maintained in RPE maintenance media (RPEMM) (MEM+glutamax, 32561037, ThermoFisher; 5% FBS, SH30071.03, Hyclone; Taurine, T-0625, Sigma; Thyronine, T-5516, Sigma; Hydrocortisone, H-0396-10, Sigma) for 15 days. RPE cells enriched with anti-CD24 (1:500, 655154, BD Biosciences) and anti-CD56 (1:500, 340723, BD Biosciences) antibodies, then seeded onto vitronectin coated transwells (3460, Corning), and cultured for 6 weeks before assays and experiments. All iPSC work was performed under institutional review board-approved protocol. All iPSC-RPE cultures were quality-controlled before use, and no cultures below a TER cut-off 400 Ohms cm² were used.

Pipette fabrication. Sharp microelectrode pipettes were pulled from fire-polished borosilicate glass with a filament (Sutter Instrument, no. BF100-50-10, 1 mm outer, 0.5 mm inner diameter) on a P-97 puller with a 2.5×2.5 mm box filament (Sutter Instrument). The pipettes were pulled in a single cycle in about 10 seconds, and the resulting pipette had a tip size between 100 nm and 200 nm in diameter (validated with scanning electron microscopy) and resistance between 120 MΩ and 250 MΩ when filled with 150 mM KCl. Pipettes with offset potential magnitudes greater than 12 mV were discarded.

Tissue mounting and maintenance. The study performed the method for mounting the tissue into the modified Ussing chamber for electrophysiology described in [32]. Briefly, a 7 mm diameter, circular section of the iPSC-RPE Transwell was punched out, mounted, and sealed into the chamber, apical side up. Electrical current leak, due to poor sealing between the Ussing chamber and the Transwell punch-out, was minimized by placing a nylon mesh insert beneath the sample so that the iPSC-RPE made contact around the entire circumference of the chamber [45]. The total exposed tissue cross-section was approximately 0.11 cm². Experimental solutions were perfused across both sides of the tissue for the entire duration of the experiment.

Perfusion rates across the apical and basolateral sides of the tissue were approximately 4.5 mL/min, were gravity-driven, and were adjusted manually using a threaded tube clamp. The Ussing chamber was coated in a hydrophobic solution (Sigmacote, Sigma-Aldrich) to induce flow through small channels in the Ussing chamber, i.e., overcome hydrodynamic forces in small cross-sectional areas of the Ussing chamber that would otherwise halt perfusion in the setup. Perfusion solution tubing was maintained at approximately 36° C. with a fabricated water jacket to prevent CO₂ from precipitating out of the solution The regulation of the temperature inside the chamber was controlled between 35.5° C. and 36.5° C. The waste solutions of the apical and basolateral baths were kept electrically isolated in two different flasks to prevent electrical shorting.

Electrophysiological solution. The standard, control solution was the same modified Ringer's solution as has been described previously [32]. In brief, the solution contains (in mM): 5 KCl, 116.5 NaCl, 23 NaHCO₃, 0.5 MgCl₂, 1.8 CaCl₂, 2 taurine, 5 glucose, and 10 sucrose. All salts (except for CaCl₂) were mixed into de-ionized water at room temperature (20 C). The solution was bubbled for approximately 5 minutes with 95% CO₂ before the addition of CaCl₂. After all salts were dissolved into the Ringer's solution, the solution had an osmolality of 295±5 mOsm/kg. During an experiment, the solution was continuously bubbled with a custom gas mixture (5% CO₂, 10% 02, and 85% N₂) to maintain the Ringer's solution to a pH near 7.4±0.1.

Measurement Setup and Electrode Configuration. To measure the apical and basolateral bath voltages, Ringer-filled 3.5% agar bridges were connected to saturated KCl baths with submerged, double-junction AgCl reference electrodes (Fisher Scientific, Accumet, cat. No. 13-620-855). AgCl-coated Ag wires (0.01″ diameter) were submerged directly into the apical and basolateral baths to apply current across the tissues during galvanostatic measurements. The Ussing chamber was rotated approximately 30 degrees from horizontal during each experiment to simultaneously enable equal fluidic pressures across the epithelia while simultaneously allowing for apical bath access for intracellular pipettes.

The study performed the protocol for inserting a pipette into the cell's cytoplasm (FIG. 5C, node 3) for intracellular recordings described in [32]. Briefly, a LabVIEW-based controller was used to insert a pipette—backfilled with 150 mM NaCl—into the cytoplasm of a single cell using an N-565.260 piezoelectric motor connected to an E-861.1A1 single-axis controller box (Physik Instrumente). The pipette voltage was amplified using a Multiclamp 700B (Molecular Devices) amplifier, and the trans-epithelial potential (TEP) signal was amplified using a VCC 600 (Physiologic Instruments). The primary output of the Multiclamp 700B had the settings: 10 mV/mV in “Membrane Potential” mode with “gain” set to 1, and the Bessel filter was set to 30 kHz.

After mounting the tissue in the Ussing chamber and successfully inserting the pipette into a cell cytoplasm, the tissue was given 30 min to reach a steady-state (i.e., baseline). Then, for approximately 8 min, the solution was switched to a different Ringer's solution that additionally contained 100 μM adenosine triphosphate (ATP) to induce well-established electrophysiological changes in RPE [33],[34]. Finally, the apical Ringer's solution was switched back to the previously described Ringer's formulation for an additional 30 min. As a result, experiments averaged a total length of 68 min.

Recording Hardware. The TEP and pipette voltages (Vₚ) were continuously recorded during each experiment at a sampling rate of 20.1 kHz using a NI USB-6356 DAQ where: TEP(t)=V₁(t)−V₂(t) and Vₚ(t)=V₃(t)−V₁(t), as a function of time t, per the notation of nodes 1, 2, and 3 in FIG. 5C. In the hardware configuration, the apical solution and apical membrane potential were measured directly by the pipette; thus, Vₐ=Vₚ. Consequently, the basolateral solution and basolateral membrane potential are calculated using V_{b}(t)=V₂(t)−V₃(t)=−(TEP(t)−Vₐ(t)).

Electrochemical impedance spectroscopy: Galvanostatic electrochemical impedance spectroscopy (EIS) measurements were also simultaneously performed with an Autolab PGSTAT204 with a FRA32M integrator module (Metrohm AG, Utrecht, Netherlands) between nodes 1 and 2 (Error! Reference source not found.5C). NOVA 2.1.5 software was used to perform frequency sweeps from 0.5 Hz to 10 kHz at iₐₚₚ=4 A, sending up to five simultaneous frequencies, “5 sines” wave type setting, spaced at five measurements per decade. This resulted in a 2 min measurement with 106 unique frequencies, logarithmically spaced. The PGSTAT reference electrode was connected to the basal side of the tissue (FIG. 5C, node 2), and the sense electrode was connected to the apical side of the tissue (FIG. 5C, node 1) through the Ringer-based agar bridges, respectively. Similarly, the working electrode was connected to the Ag|AgCl wire at node 2 and the counter electrode was connected to the Ag|AgCl wire at node 1 (FIG. 5C). The NOVA software logged the resulting complex (real and imaginary) transepithelial impedance values using the onboard integrator module per Equation 16, where ω=2πf for the frequency. f, in Hz.

$${Z}_{a b s} ( ω ) = \frac{\vert {V}_{1} - {V}_{2} \vert ( ω )}{\vert {I}_{1} - {I}_{2} \vert ( ω )} \qquad ( Eq . 16 )$$

Electronic Model Cell. Prior to taking measurements on cells, a model electrical circuit on a breadboard with known values was first used to test the precision and accuracy of the fitting algorithm to the circuit in FIG. 5C. These model cells were created with all possible permutations of resistors Rₐ, R_{b}, Rₛ=100Ω, 1 kΩ, 10 kΩ, capacitors Cₐ, C_{b}=0.1 μF, 1 μF, 2.2 μF, and R_{solA}, R_{solB}=0Ω, 100Ω, connected as shown schematically in Error! Reference source not found.C. This resulted in 3×3×3×2×2×3×3=972 possible permutations of these circuit elements. The measuring probes for the PGSTAT and Thompson clamp were directly connected to the equivalent apical bath, basolateral bath, and cell cytoplasm (nodes 1, 2, and 3 in FIG. 5C, respectively).

The range of resistance and capacitance circuit element values was selected to represent the full range of previously reported membrane properties of epithelia. The resolution of the values was selected to enable some intermediate values, logarithmically, while keeping the number of experimental permutations reasonable. The validated resistances and capacitances for each parameter are shown in Table 1.

TABLE 1

| Parameter | Possible circuit value |
|---|---|
| R_{solA} | [0.2 Ω, 0.4 Ω, 99.2 Ω] |
| R_{solB} | [0.3 Ω, 98.7 Ω] |
| Rₐ | [99.2 Ω, 988 Ω, 9790 Ω, 9837 Ω, 9842 Ω, 9850 Ω] |
| R_{b} | [0.09 μF, 1.07 μF, 2.35 μF] |
| R_{c} | [98.9 Ω, 994 Ω, 9790 Ω, 9793 Ω, 9800 Ω] |
| C_{b} | [0.09 μF, 1.01 μF, 2.34 μF] |
| Rₛ | [100 Ω, 1001 Ω, 10029 Ω] |

Experimental protocol. In the study, the tissue was mounted in the Ussing chamber at an angle ˜30° from horizontal. After successfully inserting the pipette into a cell cytoplasm, the tissue was given 30 min to reach steady state (i.e., baseline). Then, for −10 min, 100 μM of fresh ATP was dissolved into Ringer's solution, perfusing the apical side of the RPE to induce well-established electrophysiological changes (38, 39). Finally, the apical Ringer's solution was switched back to the previously described Ringer's formulation for an additional 30 min.

The combined EIS and intracellular electrophysiology methods were performed on retinal RPE cells, studying the ATP response as a function of time. The unique resistance and capacitance values in these cells were solved at each time point. Specifically, from a typical 68-minute experiment, with each measurement taking approximately 2 mins, approximately 30-time point measurements were obtained for each of the apical, basolateral, and shunt properties.

Data Analysis. In this experimental setup, the pipette voltage (Vₚ) corresponds to the instantaneous difference between the voltages at nodes 3 and 1 in Error! Reference source not found.C and is generally referred to as the apical membrane and apical solution potential, Vₐ. Therefore, the instantaneous basolateral membrane and basal solution potential (V_{b}) can be calculated using Kirchhoffs Voltage Law where: V_{b}(t)=TEP(t)−Vₐ(t).

The magnitudes of both membrane potentials (|Vₐ(ω)| and |V_{b}(t)|) were computed using a short-time Fourier transform function in Matlab (stft, Matlab, Mathworks, New Jersey) to take numerous Fourier transforms using small, Hanning windowed segments of Vₐ(t) and V_{b}(t).

To fit the parameters Rₐ, R_{b}, Cₐ, C_{b}, Rₛ, R_{solA}, R_{solB} in Error! Reference source not found.C, collectively referred to as the term βₖ, to the impedance data, Matlab's nonlinear least squares solver, lscurfit, was used with the parameters of Table 2. Specifically, Table 2 provides the validated electronic circuit parameters, denoted as β, alongside their corresponding fitted parameters, βₖ, as obtained using the exemplary method. The parameters are related to the three representative examples illustrated in FIG. 7A. Each row in the table corresponds to a specific element of the equivalent electronic circuit, as depicted in FIG. 5C.

TABLE 2

| Parameter | Value | Description |
|---|---|---|
| Algorithm | ‘trust-region-reflective’ | Optimization algorithm used |
| MaxIterations | 1 × 10⁴ | Maximum number of |
| | | iterations allowed |
| MaxFunctionEvaluations | 1 × 10⁴ | Maximum number of function |
| | | evaluations allowed |
| FunctionTolerance | Kₑ * norm(f′ − f_{measured}) | Tolerance for convergence |
| | | based on the function value |
| OptimalityTolerance | Kₑ * norm(f′ − f_{measured}) | Tolerance for convergence |
| | | based on the gradient of the |
| | | function |
| FiniteDifferenceStepSize | $max ( {eps}^{\frac{1}{3}} , min ( 1 e - 4 , {K}_{e} * norm ( {f}^{′} - {f}_{measured} ) )$ | Step size for approximating the Jacobian matrix |
| | | Scaling factors for the |
| | | parameters during |
| TypicalX | | optimization |

The upper and lower bounds of the fit were constrained to Rₐ, R_{b}, Rₛ∈[1 Ω, 5×10⁵Ω], Cₐ, C_{b}∈[1×10⁻¹³ F, 1×10⁻³ F], R_{solA}, R_{solB}∈[0 Ω, 200Ω]; selected based on the full range of previously reported membrane properties of epithelia.

To mitigate the risk of converging to local minima or boundary values, each fitting procedure was initialized 500 times with randomized starting values. The bounds and starting points were chosen to span the ranges of previously reported membrane properties in epithelial tissues.

A representative STFT result is shown in FIG. 7G.

Electronic cell results. For each of the 972 possible configurations of Rₐ, R_{b}, Cₐ, C_{b}, Rₛ, R_{solA}, R_{solB}, the study measured and modeled the circuit of FIG. 5C.

FIG. 6A shows a representative example of the complex (real and imaginary) transepithelial impedance along with the impedance ratio, as measured (X(w) Y(w) Zᵣ(w)) and modeled P(w) Q(w) R(w), respectively. It can be observed the modeled fit matched the data well qualitatively in FIG. 6A. Specifically, FIG. 6A shows three examples of the measured (▪) and fit (−) impedance data. The examples were measured at 111 frequencies between 0.5 Hz and 10 kHz, logarithmically spaced. (Example 1) Rₐ=988Ω, R_{b}=99Ω, Rₛ=10,029Ω, Cₐ=1.07 μF, C_{b}=2.34e−6 μF. (Example 2) Rₐ=9,837 Ω·cm², R_{b}=994 Ω·cm², Rₛ=10,029 Ω·cm², Cₐ=0.092 μF/cm², C_{b}=2.34 μF/cm². (Example 3) Rₐ=9,837 Ω·cm², R_{b}=994 Ω·cm², Rₛ=10,029 Ω·cm², Cₐ=1.07 μF/cm², C_{b}=0.092 μF/cm².

The study next analyzed the data for all 972 permutations to understand the fitting error across the biological range, as shown in FIG. 6B, subpanels B through subpanels E. The initial data shows the fit matched the electrical circuit values well across the entire range, with R² between 0.97 and 0.99. Thus, the exemplary method employed in the study of combined EIS and intracellular electrophysiology was able to accurately determine the resistances and capacitances of the circuit model (FIG. 5C) within biological ranges of apical, basolateral, and shunt resistances and capacitances. Specifically, FIG. 6B shows the computed circuit values matching the electrical circuit element values. Model cells were created with all permutations of resistors Rₐ, R_{b}, Rₛ=100, 1 k, 10 kΩ, capacitors Cₐ, C_{b}=0.1, 1, 2.2 μF, and R_{solA}, R_{solB}=0, 100Ω. For these resulting 972 permutations, linear plots of the fit vs. circuit board value were plotted, where the linearity is an assessment of the error. For all plots, the R² fit ranges from 0.97 to 0.99. Updated results showing that the accuracy was sensitive and specific for a range of epithelial tissues and conditions are provided in Table 3 showing quantification of median fitting error in the parenthesis (25%-75%). We explained the large R_solA and R_solB median percentage error as an artifact of normalizing on small resistance values in the surrounding text. Besides R_solA and R_solB, the median percentage error for parameters 104 were between 11-28%.

TABLE 3

| | 3 P-EIS, % | | 2 P-EIS, % | | P Value | Effect Size |
|---|---|---|---|---|---|---|
| Cₐ | 11 | (3-54) | 52 | (14-100) | 1 × 10⁻³⁷ | 0.41 |
| C_{b} | 14 | (3-57) | 50 | (14-98) | 1 × 10⁻³⁹ | 0.42 |
| Rₐ | 28 | (11-83) | 94 | (47-793) | 1 × 10⁻³⁸ | 0.42 |
| R_{b} | 26 | (7-75) | 92 | (45-530) | 1 × 10⁻⁶³ | 0.54 |
| Rₛ | 19 | (6-78) | 51 | (13-200) | 2 × 10⁻⁹ | 0.19 |
| R_{solA} | 100 | (9-952) | 96 | (46-952) | 4 × 10⁻²¹ | 0.3 |
| R_{solB} | 100 | (10-1,971) | 99 | (47-1,053) | 9 × 10⁻¹⁴ | 0.25 |

Table 3 shows the median and interquartile range of fitting errors for the exemplary system (3-electrode system) determined per Equation 13. Statistical analysis includes a one-sided Wilcoxon's paired sample test and calculation of effect size for each parameter. For reference, by looking at the 2 P-EIS median error and comparing with the biological variability in literature, sth study showed that the median and interquartile range (IQR) of the error was significant compared to normal biological variability in Table 3. 2 P-EIS is likely not practical for biological measurements of cells/tissues like RPE. In contrast, by adding the 3rd probe to the measurement, the study using the exemplary system and method can reduce the median and the IQR of the error to meaningly small ranges.

In comparison to reported biological median errors for Rₐ, R_{b}, and Rₛ calculated using alternative techniques already published in the literature [44′], [43′], [35′], [57′] (which rely on assumptions about cell function and/or drug treatment assays), it can be observed that the biological variability of that study to the maximum median error of Table 3 is greater than 28% and across almost all parameters. It can be concluded that the exemplary device is sensitive and specific for most biological assays of epithelial tissues.

Furthermore, for Rₐ, across the entire biological range of 100 to 10,000 Ω, 99.7% of all the fit values (within 3 standard deviations) are accurate. Similarly R_{b}, Rₛ, Cₐ, and C_{b} are accurate. R_{solA}, and R_{solB} are not plotted because they were held constant over the course of the experiment (mean±SD, 20±55 Ω*cm², 40±100 Ω*cm² respectively).

Indeed, 3 P-Classical (prior intracellular technique) and 2 P-EIS (extracellular probes) were insufficient for fully determining each electrical parameter (Ca, Cb, Ra, Rb, Rs, R_{solA}, RsolB). The exemplary system and method can quantify these parameters with a sensitivity and specificity that is sufficient for biological measurements of cells/tissues like RPE.

Biological cells. In the eye, light can cause ATP to increase in the subretinal space, resulting in calcium signaling due to activated apical purinergic P2Y₂ receptors [34]. At the membrane level, ATP can cause the inhibition of apical membrane K⁺ channels as well as the activation of basolateral membrane Cl− channels [4], [33]. Inhibition of apical membrane K⁺ channels reduces membrane conductance, causing the apical membrane resistance, Rₐ to increase. Analogously, activation of the basolateral membrane Cl⁻ channels can cause a decrease in the basolateral membrane resistance, R_{b}. The responses of Cₐ, C_{b}, and Rₛ were not yet well understood. Therefore, the study replicated the well-known response, as an example, to test the ability of conventional electrophysiology and the exemplary method to measure the membrane changes of the circuit shown in FIG. 5C.

Using conventional electrophysiology to observe the changes to retinal pigment epithelium in response to ATP, it had been observed that TER immediately drops once ATP exposure occurs and only recovers after the ATP is no longer present[33]. This can occur due to R_{b} changes exceeding Rₐ changes, or Rₛ decreasing, or some combination. Similarly, VDR can increase if R_{b} changes exceed Rₐ changes. VDR is not affected by Rₛ.

The study conducted conventional extracellular TER measurement for three retinal pigment epithelium samples and the intracellular electrophysiology measurement, yielding Rₐ/R_{b}. From both measurements, during ATP administration, the values increased.

Using the exemplary method, FIG. 6C shows the resistances, Rₐ, R_{b}, Rₛ, separately, revealing the specific apical, basolateral, and shunt resistances. Specifically, FIG. 6C shows membrane-specific resistances for three samples from an iPSC-RPE cell line before, during, and after 100 μM apical ATP exposure. Grey shading indicates ATP exposure.

FIG. 6D shows membrane-specific capacitances for three samples for an iPSC-RPE cell line before, during, and after 100 μM apical ATP exposure. Grey shading indicates ATP exposure. In FIG. 6D, sample “3” shows an anomalous reading during ATP administration. TER appears as expected, but Rₐ/R_{b} does not show the expected increase. The exemplary method, which can elucidate the membrane-specific values, can provide insight. Surprisingly, Rₐ was observed to have a reversal in the expected direction of the resistance changes under ATP, while Rₛ and R_{b} appear typical. The apical membrane resistance in samples “2” and “3” increased by 89% and 139%, respectively, while the apical membrane in sample “1” decreased by 62%. From sample “3,” as compared to samples “1” and “2,” the membrane-specific values can reveal unexpected, and perhaps undesirable, changes to stimuli.

It is noted that Rₛ is not constant when ATP is applied, undermining assumptions used by Awayda [27] that Rₛ stays constant over the course of an experiment. Assumptions that rely on Rₛ being constant does not appear valid. Cottrill et al. have suggested that TER/Rₛ=0.71 for human bronchial epithelia [26]. The instant study found that TER/Rₛ ranged from 8 to 11 for RPE, and furthermore, the ratio did not remain constant during ATP exposure. Thus, the study found that the assumption can overlook up to certain errors, making the Bridges assumption off.

FIG. 6E shows the capacitances, Cₐ, C_{b} for retinal pigment epithelium in response to ATP. Both show changes during ATP administration (gray bar). It can be observed Cₐ and C_{b} changed by 12% and −6%, respectively.

Comparison to VDR and TER. The TER and VDR are computed as comparisons for electrophysiological assessment of epithelial tissues.

Classically, intracellular electrophysiology data are analyzed using a term called the voltage divider ratio (VDR). VDR represents the approximate ratio between the apical and basolateral membrane resistances. The VDR overcomes the limitation of traditional intracellular electrophysiology setups that cannot separately monitor transcellular and shunt current. Without a direct, simultaneous measurement of both voltage and current through the cytoplasm of the cell using the intracellular electrode, it is impossible to solve for the apical, basolateral, and shunt resistances using Ohm's law.

To calculate the VDR, the voltage change generated by an extracellular, alternating current is applied at very low frequencies (e.g., f≤0.5 Hz). Furthermore, the VDR assumes the apical and basolateral membrane currents (iₐ and iₐ, respectively) to be identical, and the surrounding solution and background resistances R_{solA} and R_{solB} to be negligible relative to Rₐ and R_{b}. Consequently, by measuring the magnitude of the voltage change across the apical and basolateral membranes, the VDR provides an approximation for the ratio of the apical to basolateral membrane resistances [43], [44] as

$$VDR ( ω = π \frac{rad}{s} ) = \frac{\vert {V}_{a} ( ω ) \vert / \vert {i}_{a} ( ω ) \vert}{\vert {V}_{b} ( ω ) \vert / \vert {i}_{b} ( ω ) \vert} ≅ \frac{{R}_{a}}{{R}_{b}} .$$

Extracellular electrophysiology parameters, as shown in FIG. 1B, are often reported as a single transepithelial resistance (TER), which is calculated using Ohm's law, V=IR, where V is the potential difference, I is the electric current, and R is the electrical resistance, at similarly low frequencies (f≤0.5 Hz). Consequently, traditional TER is measured as

$$TER ( ω = π \frac{r a d}{s} ) = \frac{\vert TEP ( ω ) \vert}{\vert {i}_{applied} ( ω ) \vert} = {R}_{s o l A} + {R}_{s o l B} + \frac{{R}_{s} ( {R}_{a} + {R}_{b} )}{{R}_{s} + {R}_{a} + {R}_{b}} ,$$

$$where \vert {i}_{a p p l i e d} ( ω ) \vert$$

is the magnitude of the applied current.

Representative examples of impedance data. FIG. 7A can be visually inspected to qualitatively evaluate the electrical properties of an epithelial tissue. For example, the magnitude of the real impedance (FIG. 7A, top row) at the lowest measured frequencies represents the sum of the TER, apical, and basolateral solution resistances (R_{solA} and R_{solB}, respectively). Additionally, the real impedance at the highest measured frequencies represents the resistance of only the apical and basolateral solution resistances (R_{solA} and R_{solB}, respectively). By evaluating the real impedance (X(ω) at both high and low frequencies, a more accurate representation of the integrity of the epithelial tissue can be extracted by decoupling the solution and growth substrate resistances (R_{solA} and R_{solB}) from the TER.

When the cell membranes are modeled with a resistor in parallel with a capacitor, as is shown in FIG. 5C, the imaginary impedance (FIG. 7A, middle row), can reveal information about the time constants, r, of the apical and basolateral membranes. In a general sense, the time constant of a cell membrane represents how quickly it can re-equilibrate after a sudden change in transmembrane voltage or current. This parameter can be calculated using the formula, τ=RC.

An interesting feature of most epithelial tissues is that they are polarized, meaning that they have different electrical transport and functional pathways in their apical and basolateral membranes [46], [47], [48], [49]. Consequently, epithelia may have distinctly different apical and basolateral time constants [50]. For example, if the cells are stimulated by ATP (see Results), the apical and basolateral membranes of RPE typically have unique and opposite electrical responses. These unique and opposite responses of r could be visualized as diverging or converging minimums in the imaginary impedance plot.

The membrane ratio (FIG. 7A, bottom row) is the only property that measures the ratio of apical to basolateral impedance. At the lowest measured frequencies (ω→0 rad/s), the system asymptotically approaches the limit,

$${lim}_{w \to 0} ( {R}^{′ ( {ω}_{i} )} ) = \frac{{R}_{a} {R}_{s} + {R}_{s o l A} ( {R}_{a} + {R}_{b} + {R}_{s} )}{{R}_{b} {R}_{s} + {R}_{s o l B} ( {R}_{a} + {R}_{b} + {R}_{s} )} ,$$

which simplifies to the VDR from equation (1) when R_{solA} and R_{solB} are much less than the apical and basolateral resistances, and can be set to approximately 0 [33′], [34′]. Conversely, at high frequencies (ω→∞ rad/s), the membrane ratio approaches the following limit:

$${lim}_{w \to ∞} ( {R}^{′ ( {ω}_{i} )} ) = \frac{{R}_{s o l A}}{{R}_{s o l B}} .$$

To demonstrate these qualitative metrics, they were applied to the representative data shown in FIG. 7A. The exact values measured by the digital multimeter, βk, for each circuit parameter k and corresponding parameter fits, β′ₖ are shown in FIG. 7D. Specifically, FIG. 7D enumerates the specific equations and parameter values configured for Matlab's ‘optimoptions’ function, which were subsequently applied to the ‘lsqcurvefit’ algorithm for curve fitting. The settings detailed in this table are the non-default options utilized in the study and were selected based on guidelines provided in Matlab's SimBiology documentation pertinent to biological data fitting.

In FIG. 7A, the exact TER (total epithelial resistance plus the solution resistances) for examples 1, 2, and 3 were 720 Ω, 1750Ω, and 5180Ω, respectively. These correlated well with the values that can be calculated by inspecting the low-frequency asymptote of the real impedance traces shown in FIG. 7A: 720 Ω, 1750Ω, and 5160Ω, respectively. Furthermore, the exact sum of solution resistances for examples 1, 2, and 3 in FIG. 7A were 198 Ω, 99Ω, and 198Ω, respectively. These correspond well with the high-frequency asymptotes of FIG. 7A: 210 Ω, 135Ω, and 278Ω, respectively.

The ratio of apical to basolateral time constants in FIG. 7A example 1 and example 2 are nearly identical (approximately 25:1). Visual inspection of the imaginary impedance traces, therefore, should have 2 minimums. The difference in FIG. 7A, example 2 is clear but in example 1 appears only as a “slowing” of the return to 0Ω for the imaginary impedance around 1 kHz. This “slowing” phenomenon shows the impact of the lower shunt resistance (Rₛ), attenuating the visible changes in the imaginary impedance data. Furthermore, example 3 in FIG. 7A has a ratio of time constants 1:2600. Notably the second minimum likely above the 10 kHz measurement range and is not visible in the trace. This assumption can be made by visual inspection of the imaginary impedance data because the measured data appears to be diverging away from 0Ω at 10 kHz, hinting that another minimum may exist at higher frequencies. Despite the lack of clear visual evidence of distinct minimums in examples 1 and 3 from FIG. 7A, the calculated membrane time constants were still accurate when using the exemplary method. Namely, 24:1, 26:1, and 1:620 for examples 1, 2, and 3, respectively.

Finally, visual inspection shows the asymptotes of the membrane ratio (FIG. 7A bottom row) of noted limits. Therefore, in FIG. 7A, the low frequency asymptotes should approach 3.9, 0.9, and 0, for examples 1, 2, and 3, respectively, which it appears to do. Similarly, the high-frequency asymptotes in the membrane ratio in FIG. 7A should be 1, 0, and 1 for examples 1, 2, and 3, respectively, which it appears to do.

Quantification of residual error. FIG. 7A offers compelling evidence for the model's of the exemplary method aligns with the measured data across diverse electronic circuit configurations. The residual error, r(ω)=|d′(ω)−d(ω)|, underscores good model correlation, marked by medians and interquartile ranges (IQRs) of 1.7Ω (1.0-4.1), 0.8Ω (0.3-2.0), and 0.0 (0.0-0.1) (n=103,032). The residual was low across the extensive dataset comprising 972 circuits measured at 106 frequencies, resulting in a total of 103,032 data points. Additional insights into the residual distribution are shown in FIG. 7H. The minimal residual errors substantiate the robustness and reliability of the model in the exemplary method in accurately capturing the real impedance, imaginary impedance, and membrane ratio. Furthermore, the near-zero median residual error highlights the model's potential efficacy in discerning subtle, clinically pertinent variations in membrane properties.

Parameter estimation error. The exemplary method used in the study enables the assumption-free measurement of electrical properties in epithelial tissues. To quantify this error, the relative difference between the known circuit value, βₖ and the calculated value, β′ₖ, was inserted into

$${ε}_{k} = \frac{\vert β {′}_{k} - {β}_{k} \vert}{{β}_{k}} \times 100$$

as the parameter estimation error.

The exemplary method significantly reduced parameter estimation error compared to a control model, referred to as 2P-EIS. In the 2P-EIS model, the extracellular EIS data—both real impedance and imaginary impedance—were fit to the circuit model identical to the model of the exemplary method. That is, the intracellular data corresponding to node 3 in FIG. 5C were deliberately excluded in the 2P-EIS analysis. In assessing biologically significant resistances, namely Rₐ, R_{b}, and Rₛ, the data presented in FIG. 7E indicate an average estimation error of 24% (SD 4, n=3) using the exemplary method. Specifically, FIG. 7E provides results of a comparison between the median and interquartile range (IQR) of fitting errors between the exemplary method and the 2P-EIS method, calculated as per equation

$${ε}_{k} = \frac{\vert β {′}_{k} - {β}_{k} \vert}{{β}_{k}} \times 100 \qquad ( n = 9 \; 72 ) .$$

Statistical analysis includes a one-sided Wilcoxon's paired sample test and calculation of effect size for each parameter. The aim of the statistical comparison is to evaluate the significance and the effect size of the differences between the two models. Further graphical representation of these results is shown in FIG. 7I.

This is a substantial improvement over the 79% (SD 20, n=3) error observed with the control model 2P-EIS. When compared with the average biological error of 51% (SD 22, n=27) compiled from values reported in previous studies (FIG. 7F), the practical utility of exemplary method seems clear. Specifically, FIG. 7F shows a comparative analysis of the average errors in measuring apical, basolateral, and shunt resistances in epithelial tissues, based on published literature. The errors are represented as percentages, calculated using the mean (μ) and standard error (SE) via the formula: error (%)=100√n(SE)/μ. The ‘Source’ column specifies the author(s) along with the citation number, the tissue type investigated, and the measurement technique employed. Except for the ‘multiple intracellular electrodes’ method, all techniques summarized below involve modulating the tissue electrophysiological state with a stimulus (e.g., glucose), while assuming the stimulus affects only a subset of the resistances. The resistances are then calculated using electrical properties measured in two or more states and a corresponding set of equations.

To disentangle the biological uncertainty from the hardware and measurement techniques an analysis of variance (ANOVA) on existing data recorded from one epithelial tissue type, using three independent techniques, can be performed. Specifically, the three different techniques deployed by Bello-Reuss to measure Rₐ, R_{b}, and Rₛ for Ambystoma tigrinum have an ANOVA P-value of 0.8, and a minimum Tukey honestly significant difference (HSD) post-hoc test P-value of P=0.8 between the glucose and Ba2+ groups. This indicates that variations in parameter magnitudes between experiments are likely caused by typical levels of biological variability rather than measurement technique(s) and hardware.

Beyond the biologically relevant resistances, the exemplary method can also, simultaneously, measure solution resistances R_{solA} and R_{solB}. In FIG. 7E, the median error of the exemplary method (shown as “3P-EIS”) is notably larger than the other circuit parameters. However, this is an artifact of calculating a normalized error on very small resistances (<0.5Ω in 5). On an absolute error scale (i.e., not normalized by the magnitude of the known parameter, βₖ), R_{solA} and R_{solB} had median and IQR error of 7Ω (1Ω-9Ω) and 8Ω (1Ω-21Ω), respectively, when fitting using 3P-EIS. Besides this error being very small relative to the magnitude of the other resistances tested in this paper, solution resistances are typically measured prior to a practical electrophysiology experiment and do not provide pertinent information when trying to understand the electrophysiological properties of an epithelial tissue. However, the ability to monitor these resistances throughout—as opposed to before—an experiment can be beneficial. For example, when the solution resistance changes or when the cell growth substrate degrades throughout an experiment.

Although capacitance is defined by C=εA/L, where E is a dielectric constant, A is surface area, and L is membrane thickness, it is generally assumed that E and L are constant for cell membranes. Further, the ratio of E/L is similar in magnitude across many cell types. As a result, the cell capacitance, C, is generally accepted to be around 1 μF/cm2 and alterations in measured capacitance most likely indicate changes in membrane surface area [32′], [52′], [60′], [61′]. To understand the typical variance in cell capacitance in physiological experiments, it is instructive to examine existing literature. For example, both Weber et al. [62′] and Takahashi et al. [63]′ reported 3′,5′cyclic monophosphate (cAMP)-induced increases in Xenopus laevis oocyte membrane capacitance, ranging from 12-60%. From FIG. 7E, the exemplary method (3P-EIS) has a median error of 11% for 521 Cₐ and 14% for C_{b}, with respective IQRs of 3-54% and 3-57%. Given this distribution, the system is likely sensitive enough to detect changes of the magnitudes reported by Weber and Takahashi, albeit with caveats. Specifically, the skewed error distribution implies that while the system may generally be reliable, there are instances where significant errors could occur. These findings, therefore, should be interpreted cautiously, particularly when approaching the boundaries of this reported range of physiological changes.

While the model of the exemplary method appears to have sufficient sensitivity for most biological experiments on epithelial tissues, it is not without some shortcomings. For instance, each measurement is two minutes, and, following Nyquist's sampling theorem, cannot be used to measure dynamic processes that change with a period less than four minutes; effectively steady state. An example of a response that is missed by 3P-EIS is the initial phase of the ATP response in the TEP data (FIG. 7B, panel A). Potential solutions to this limitation include reducing the number of frequencies required per decade, decreasing the number of cycles needed to capture each frequency, or using more complex signals such as chirps that quickly sweep through a large frequency range [64′]. The stability of the fitting algorithm also has its limits. In 265 out of the 927 fits, one of the membrane resistance parameters (Rₐ, R_{b}, Rₛ) was estimated to be within 1% of the maximum value constrained by the fitting algorithm (termed “railing”). In contrast, the capacitances and solution resistances (Cₐ, C_{b}, R_{solA}, and R_{solB}) did not rail. While there is not a singular cause for this railing condition, it only occurs in one of the membrane resistance parameters at a time. In other words, if Rₐ railed, then R_{b} and Rₛ did not, and so on. Furthermore, the impact on the estimation error for a particular railing condition was dependent on which parameter railed. For instance, when Rₐ railed, the parameter estimation errors were relatively unchanged. However, when R_{b} railed, Cₐ, Rₐ, and R_{solA} parameter estimation error increased, matching the median error for 2P-EIS. Finally, when Rₛ railed, the magnitude of all other parameter estimation errors resembled that of 2P-EIS. This increase in error suggests that the sensitivity of the remaining parameters is reduced in cases with “railing.” The effect of railing on the remaining circuit parameters is shown in Table 3.

TABLE 3

| Parameter | Rₐ (n = 79) | | R_{b} (n = 45) | | Rₛ (n = 141) | |
|---|---|---|---|---|---|---|
| Cₐ | 3 | (2-6) | 49 | (18-85) | 21 | (7-88) |
| C_{b} | 12 | (5-39) | 9 | (3-19) | 22 | (6-56) |
| Rₐ | — | | 20 | (7-58) | 50 | (16-89) |
| R_{b} | 12 | (6-26) | — | | 52 | (20-72) |
| Rₛ | 9 | (8-50) | 10 | (5-18) | — | |
| R_{solA} | 18 | (3-100) | 2154 | (15-3492) | 100 | (14-808) |
| R_{solB} | 4 | (1-962) | 9 | (1-100) | 141 | (36-5667) |

The mathematical model of the exemplary method has demonstrated the ability to accurately determine the independent apical, basolateral, and shunt resistances and capacitances of the circuit model (FIG. 5C) within biologically relevant ranges with a biologically-useful degree of accuracy. This highlights the value of an exemplary method for characterizing the electrical properties of epithelial tissues, offering new insights into biological systems and the potential for further advancements in drug discovery, channels/pathway experiments, and quality control metrics.

Demonstration on biological samples. To biologically validate the mathematical model presented in this paper, the response of three RPE tissues to the apical application of 100 M ATP was measured over a one-hour period using both 3P-Classical (measuring TER and VDR at one, low frequency) and the exemplary method. The ATP response of iPSC-derived RPE tissues (from two donors) was selected for this validation because the ATP pathway has been thoroughly tested on primary bovine, human RPE, and iPSC-derived RPE cells (7, 38, 39). In short, in the eye, light causes ATP to increase in the subretinal space, resulting in intracellular RPE Ca2+ signaling triggered by activated apical purinergic P2Y2 receptors [38′]. The increase in intracellular Ca2+ causes the inhibition of Ca2+-sensitive apical membrane K⁺ channels as well as the activation of Ca₂+-sensitive, basolateral membrane Cl-channels [7′], [39′]. Inhibition of apical membrane K⁺ channels reduces membrane conductance, causing the apical membrane resistance, Rₐ to increase. Analogously, activation of the basolateral membrane Cl− channels causes a decrease in the basolateral membrane resistance, R_{b}. Although Rₐ and R_{b} are well understood in this paradigm, the responses of Cₐ, C_{b}, and Rₛ are not yet well understood.

Epithelial voltages. To begin, the TEP and apical voltage (Vₐ) were continuously recorded and can be used to begin the interpretation of the ATP-evoked response for both the 3P-Classical method and the exemplary methods (FIG. 7B, panel A). The top row of FIG. 7B, panel A shows the TEP data and reveals the difference in the apical and basolateral membrane voltages [31′], [55′], [57′], [65′]. Sample 3 had an opposite TEP polarity at baseline and is missing the first phase TEP decrease (within the first minute) of the ATP-evoked response compared to Samples 1 and 2. All three samples showed an increase in TEP over the remainder of the ATP exposure and recovered to their original values after returning to normal Ringer's solution.

The bottom row of FIG. 7B, panel A shows the intracellular voltage data measured with the pipette, which corresponds to the apical membrane potential (Vₐ). The potential recorded by the pipette before and after apical ATP is normal (e.g., <−45 mV) and relatively stable in Sample 1, 2, and 3 in the standard Ringer's solutions [7′], [38′], [65′], [66′]. The depolarization of the apical membrane in the first phase of the ATP-induced response was present yet smaller for Sample 3 compared to Samples 1 and 2 (10 mV compared to 20 mV, respectively). However, all three samples depolarized by approximately 20 mV by the end of the ATP exposure.

Epithelial resistances. FIG. 7B, panel B shows the TER and VDR of the same samples in FIG. 7A, panel A. These parameters were calculated using two different methods: 3P-Classical and the exemplary method. The TER and VDR from the 3P-Classical analysis were calculated by inserting the estimated parameters (R_{solA}, R_{solB}, Rₐ, R_{b}, and Rₛ) into the equation,

$$TER ( ω = π \frac{r a d}{s} ) = \frac{\vert TEP ( ω ) \vert}{\vert {i}_{applied} ( ω ) \vert} = {R}_{s o l A} + {R}_{s o l B} + \frac{{R}_{s} ( {R}_{a} + {R}_{b} )}{{R}_{s} + {R}_{a} + {R}_{b}} .$$

The TER and VDR from the model of the exemplary method were calculated by inserting the estimated parameters (R_{solA}, R_{solB}, Rₐ, R_{b}, and Rₛ) into

$$V D R ( ω = π \frac{r a d}{s} ) = \frac{\vert {V}_{a} ( ω ) \vert / \vert {i}_{a} ( ω ) \vert}{\vert {V}_{b} ( ω ) \vert / \vert {i}_{b} ( ω ) \vert} ≅ \frac{{R}_{a}}{{R}_{b}} .$$

The results presented in FIG. 7B, panel B (top row) show nearly identical TER using either the 3P-Classical or the exemplary method. Both methods depict a decrease in TER during ATP exposure and some recovery after ATP removal, consistent with previous reports [7′], [38′], [39′]. However, TER measurements alone cannot distinguish between changes in Rₐ, R_{b}, and Rₛ. To gain more insight, VDR was also measured (FIG. 7B, panel B, bottom row), and additional information about the relative changes in the membrane-specific resistances (Rₐ and R_{b}) can be evaluated.

In FIG. 7B, panel B, there are discrepancies in magnitude between the VDR obtained through the exemplary method and the 3P-Classical method during ATP stimulation for Samples 1 and 2, as well as 599 across all tests for Sample 3. The key discrepancy arises from the limitations of sampling at a single, discrete frequency (0.5 Hz) in 3P-Classical, which sometimes fails to accurately approximate the membrane ratio's asymptote as it approaches 0 Hz. Unlike 3P-Classical, which requires near-zero frequencies to accurately account for all capacitive effects, the exemplary method offers significant flexibility by allowing for data extrapolation to theoretical VDR values at 0 Hz. This extrapolation advantage enables the exemplary method to perform robustly in dynamic conditions requiring faster sampling rates, delivering superior VDR accuracy and even capturing dynamic changes in VDR that could be missed with 3P-Classical (e.g., the change in VDR during apical-ATP for Sample 3). Moreover, the exemplary method can sidestep the inherent unpredictability of the 3P-Classical method, which cannot pre-determine the optimal frequency for precise VDR measurements. Further details on real and imaginary impedance and membrane ratios are shown in FIG. 7J.

Unique to the exemplary method (compared to 3P-Classical), the solution resistances, R_{solA} and R_{solB}, were calculated using the model of FIG. 5C. The solution resistances were relatively consistent magnitude before, during, and after ATP exposure. Consequently, the average magnitude of these values was 3 (SD 3)Ω×cm2 and 9 (SD 1)Ω×cm2 for R_{solA} and R_{solB}, respectively (n=85). The elevated R_{solB} is likely due to the extra resistance of the growth substrate. Furthermore, the magnitude of the sum of these resistances corresponds well with historical measurements of the resistances of empty growth substrates installed in the modified Ussing chamber, submerged in Ringer's control solutions, typically between 10-20Ω×cm². The ability to monitor the solution resistance during an experiment is an important improvement over 3P-Classical. For example, when cells are grown on biodegradable scaffolds or when cells secrete large amounts of extracellular matrix, R_{solB} may increase or decrease in a manner that is difficult to compensate for, masking subtle changes in electrical parameters between samples.

The exemplary method offers a continuous, assumption-free approach to quantifying all circuit parameters outlined in FIG. 5C, as demonstrated in FIG. 7C. Focusing initially on apical, basolateral, and shunt resistances (Rₐ, R_{b}, and Rₛ, respectively), Sample 3 from donor B exhibits a markedly elevated baseline apical resistance (Rₐ) of approximately 11 kΩ×cm². This contrasts with 1.6 kΩ×cm² and 3.3 kΩ×cm² for Samples 1 and 2, respectively. One possible explanation for this observation, based on recent literature, indicates that stem cell-derived RPE lines have heterogeneous densities of K⁺ channels between neighboring cells in a single epithelial tissue. This heterogeneity phenomenon can provide an explanation for the elevated Rₐ in Sample 3 [67′]. A lower number of K⁺ channels on the apical membrane would correspondingly result in higher resistance and a diminished ATP response, as is observed in Sample 3. Conversely, the basolateral resistance (R_{b}) remains comparable to that of Samples 1 and 2, and it shows the expected reduction during ATP exposure, likely due to the activation of basolateral Ca₂+-sensitive Cl− channels. Finally, across all samples, the shunt resistance (Rₛ) depicts a rapid and substantial decrease during ATP exposure and has a long recovery back to baseline.

The exemplary method proves to be especially useful when epithelial responses involve complex interactions between membranes, as is the case with typical ATP responses in RPE [7′], [38′], [39′]. Unlike 3P-Classical, which struggles to independently measure membrane resistances, the exemplary method can distinguish the atypical apical membrane response from the typical basolateral membrane response, as illustrated in Sample 3. This allows for a nuanced understanding of cellular behavior in different experimental conditions not possible with 3P-Classical.

Epithelial capacitances. Utilizing the exemplary method to calculate membrane-specific capacitances (Cₐ and C_{b}) from in FIG. 5C, the data consistently reveals an apical-to-basolateral capacitance ratio of approximately 2:1, across all samples, in control Ringer's solution, both pre- and post-ATP stimulation. This suggests that the apical surface area of RPE cells may be twice as large as the basolateral area, aligning with prior literature reports indicating a 4:1 ratio of surface areas for rat RPE [2′], [61′], [68′].

Investigation of the membrane-specific ATP-induced capacitance changes (FIG. 7C) show a difference between Sample 1 compared to Samples 2 and 3. Sample 1 did not display ATP650-induced changes. In contrast, Samples 2 and 3 experienced concurrent reductions in apical capacitance and elevations in basolateral capacitance during apical ATP stimulation. These changes could potentially signify a reduction in apical membrane microvilli and increased lateral intercellular spaces (corroborated by a reduction in the shunt resistance), respectively. However, it is also possible that, if conformational molecular changes occur during ATP exposure, an alternative explanation for changes in the apical membrane could be modifications in membrane composition, specifically membrane thickness or dielectric constant [38′], [52′], [60′], [69′].

Using 3P-EIS to test assumption-based methods during ATP administration on RPE cells. Data acquired using 3P-EIS can be used to test the validity of assumptions used in alternative methods to fully solve for all membrane parameters, βₖ, in FIG. 5C. For starters, FIG. 7C clearly shows that all membrane resistances change (Rₐ, R_{b}, and Rₛ) change when ATP is applied, undermining assumptions leveraged by previous groups where at least one membrane must remain constant to fully solve for these parameters [35′], [43′]-[45′], [57′]. Another assumption leveraged to fully determine βₖ used measurements from previous experiments of a particular wild type control cell line and asserted that the ratio of transcellular to shunt resistance was constant, even during specific drug exposures [41′]. However, the instant data clearly shows that this ratio ((Rₐ+R_{b})/Rₛ) is not constant and was dramatically different for Sample 3 compared to 1 and 2 (see FIG. 7K for details). Additional measurements are shown in FIGS. 7L-7N.

As such, traditional approaches risk overlooking or misinterpreting meaningful variations in epithelial tissues by relying on these assumptions. The exemplary method is devoid of such limitations, providing a more resilient and assumption-independent approach. This enables comprehensive evaluations of how epithelial transport pathways are affected across various scenarios, including drug treatments, disease conditions, and quality control measures.

Discussion The exemplary method can provide a more precise readout of epithelial physiology and enhance the details extracted when studying novel pathways diseases. It also, potentially, provides dynamic readouts of cell morphology without a microscope or other destructive techniques. The utility of the exemplary method extends beyond basic research. In the clinical realm, this method offers a new avenue for drug testing where membrane-specific changes or interactions are underdetermined when using 3P-Classical or in assumption based-methods that assume normal cell function. Further, the exemplary method could be used as a higher sensitivity quality control metric in transplantation therapies.

### Example #2—Epithelial Electrophysiology Using Intracellular Robotics and Extracellular Impedance Spectroscopy

A second study was conducted, preceding the first, to develop a mathematical model to measure the previously unobservable apical and basolateral membrane properties using extracellular electrochemical impedance spectroscopy.

Equations for analysis assuming very low measurement frequencies (i.e., in time domain): Despite utilizing an intracellular pipette in the modified Ussing chamber (B. A. Hughes, S. S. Miller, D. P. Joseph, and J. L. Edelman, “cAMP stimulates the Na+-K+ pump in frog retinal pigment epithelium,” Am J Physiol, vol. 254, no. 1 Pt 1, pp. C84-98, 1988.), the apical and basal membrane resistances (Rₐ and R_{b}, respectively) cannot be directly measured since it is that it is not possible to simultaneously monitor the magnitude of current and voltage flowing through the apical and basolateral membranes with a single pipette. However, if the pipette is used to monitor the intracellular voltage with respect to the apical bath (Vₐ), the external sense and reference electrodes monitor TEP (Vₐ and V_{b} Agar bridges), and the working and counter electrodes control the net transepithelial current Iₐₚₚ (Iₐ and I_{b} agar bridges), the instantaneous value of the basolateral voltage (V_{b}) can be calculated by rearranging the TEP equation (TEP=V_{b}−Vₐ). Therefore, the magnitude of the current flowing through Rₐ must be equal to the current in R_{b} from Kirchoff's Current Law, and the following resistance ratio can be calculated as a function of membrane voltages,

$$a = \frac{{R}_{a} + {R}_{s o l A}}{{R}_{b} + {R}_{s o l B}} = \frac{{V}_{a}}{{V}_{b}} ,$$

where a is frequently referred to as the voltage divider ratio.

Analysis shows the apical and basolateral membranes of the epithelia contain an equivalent EMF (Eₐ or E_{b}) in series with a resistor, Rₐ or R_{b}, respectively. The paracellular pathway (comprised of the parallel combination of tight junctions and mechanical seal between the epithelia and chamber walls) is represented as a shunt resistor, Rₛ. This shunt resistance pathway, in combination with active transport processes in epithelia, generates a short circuit current (Iₛ) that flows in a loop around the circuit. Thus, the application of Kirchhoff's voltage law can yield the following differential equations for Vₐ and V_{b} per Equation Set 16.

$${V}_{a} + \frac{{C}_{a} {R}_{a} {d V}_{a}}{d t} = {R}_{a} ( {I}_{a p p} + {I}_{s} ) - {E}_{a} + {I}_{a p p} {R}_{s o l A} \qquad ( Eq . Set 16 )$$

$${V}_{b} + \frac{{C}_{b} {R}_{b} {d V}_{b}}{d t} = - {E}_{b} - {R}_{b} ( {I}_{a p p} + {I}_{s} ) - {I}_{a p p} {R}_{s o l B}$$

$$where$$

$${I}_{s} = \frac{{V}_{b} - {V}_{a}}{{R}_{s}}$$

If the system is allowed to reach steady-state (dV/dt=0), the previous differential equations can be simplified to: Vₐ=Rₐ(Iₐₚₚ+Iₛ)−Eₐ+IₐₚₚR_{solA} and V_{b}=−E_{b}−R_{b} (Iₐₚₚ+Iₛ)−IₐₚₚR_{solB}. Furthermore, if no current is applied by the external hardware (Iₐₚₚ=0), then Vₐ=IₛRₐ−Eₐ and V_{b}=−E_{b}−IₛR_{b}. When the current flowing through the circuit is allowed to reach a steady state, then the current that flows through the capacitors is necessarily 0. Thus, the circuit for intracellular electrophysiology can be reduced to the form shown in FIG. 1.6C. For this circuit, the total tissue resistance (Rₜ) can be calculated using conventional rules for combining resistors in parallel as:

$${R}_{t} = {R}_{s o l A} + {R}_{s o l B} + \frac{{R}_{s} ( {R}_{a} + {R}_{b} )}{{R}_{s} + {R}_{a} + {R}_{b}} .$$

Electrophysiologists have used the voltage divider ratio (a) and the total tissue resistance (Rₜ) to make assumptions about which membranes are responding to a stimulus. For example, if a concomitant decrease in a and an increase in Rₜ is recorded, then it is most likely that R_{b} increased. However, it is also possible that R_{b} was constant and Rₐ decreased if Rₛ increased sufficiently to obfuscate the results in the measurement of Rₜ. Therefore, to conclude that the dominant contributor to the observed change in a and Rₜ was R_{b}, the experimenter must perform a follow-up test that targets the proposed mechanisms in the basolateral membrane to inhibit the changes. If the changes can be blocked with a drug known to target the proposed mechanisms on the basolateral membrane, only then is it safe to conclude that the observed changes in a and Rₜ are due to R_{b}.

To analyze a circuit with capacitors, it is useful to model all circuit elements using the formal impedances (Z) that are comprised of a real and imaginary component. These impedance terms consider the complex behavior of circuit elements in response to a general AC input signal; often represented as a sinusoid (A sin wt+ϕ). This more general formulation of an input signal is a function of amplitude A, frequency w (in rad/s), time t, and phase shift ϕ. For the simple case of a resistor, the complex impedance is directly proportional to the magnitude of the real circuit resistance (R) such that Z_{R}(w)=R.

Workflow. The values for Rₐ, R_{b}, Rₛ, Cₐ, and C_{b} were calculated in a post-processing algorithm, developed in Matlab 2021b.

EIS measurements were performed with an Autolab PGSTAT204 with a FRA32M integrator module (Utrecht, Netherlands). The device was controlled with the provided NOVA 2.1.5 software to perform frequency sweeps from 0.1 Hz to 10 kHz. The NOVA software was used to send up to 5 simultaneous frequencies spaced to approximately 5 unique measurements per decade. The PGSTAT working electrode was connected to the working electrode and counter electrode; the sense and reference electrodes were connected to the basal and apical baths through the Ringer-based agar bridges (connected to Ag|AgClreference electrodes in a saturated KCl bath), respectively.

Data processing. Because of specific hardware configurations, the raw Vₚᵢₚₑₜₜₑ and TEP data were captured during each frequency sweep. The data was collected with an NI USB-6356 DAQ at 20.1 kHz (just above the Nyquist frequency). Each frequency sweep was saved to a unique text file and stored with the measurements recorded by the NOVA software for the transepithelial impedance data. At the end of each experiment, there was a folder that contained the raw data for each recording, combined with the raw data collected by the PGSTAT204 device.

The Matlab script used to fit the raw data used the following pseudo-code: (i) load raw data into memory, (ii) append relevant metadata (e.g., date, cell line, passage number, etc.), (iii) generate an array of random initial guesses for raw data, (iv) normalize measured values to fit within the range of 0→1, (v) fit the data using the “lsqcurvefit” function available in Matlab, (vi) repeat until all recordings have been processed, and (vii) store resulting data in a new, summary text file.

Model cell circuit. The magnitude of the resistors and capacitors was selected to be similar in magnitude to the expected values of RPE, however, the exact values were unknown at this point. In total, 6 unique values for Rₐ were tested while the remaining parameters were constant.

For each experiment, the amplitudes of the voltage response for the apical and basolateral membranes were extracted from the raw data using a power spectral density (PSD) plot and windowing. Specifically, the measurement start and end times of each frequency were stored in a metadata file. Thus, with a PSD, each time window corresponding to the desired frequency could be extracted from the plot, and the power could be calculated by finding the peak value within the window.

Using the extracted amplitudes for the impedance ratio at each distinct frequency, the impedance ratio (z) and Nyquist diagram were fit using the Matlab code. The impedance ratio was checked by looking at the limits as w→0 and w→∞. The actual voltage divider ratio was approximately 0.8, and the solution resistance ratio was approximately 0.2. The left and right asymptotes of the plot appear to be trending to the limits.

To generate the data visualized in a Nyquist diagram, the tissue is measured at many distinct frequencies by clamping an alternating current across the epithelia. At each frequency, the amplitude and phase of the resultant voltage were measured, and the relationship between input and output signal was represented by the complex impedance (Z= [symbol] +i [symbol] ). The magnitude of the real and imaginary components at each distinct frequency were plotted as a discrete point in a graph where the x-axis is the real component ( [symbol] ), and the y-axis is the imaginary component ( [symbol] ). A collection of these points—each a distinct frequency—was collected as the Nyquist diagram.

Immunostaining. The study performed the protocol for immunostaining iPSC-derived RPE monolayers as described in [22′]. In short, after an experiment was completed, the Transwell punch-out (7 mm in diameter) was fixed in 4% paraformaldehyde (Electron Microscopy Science, 157-4-100) for 12 min. The sample was then rinsed three times, over the course of 15 min, with PBS at room temperature and stored at 4° C. Immunohistochemistry blocking solutions (IBS) consisted of 500 mL of 1× Dulbecco's phosphate-buffered saline (DPBS, Line Technologies, 14190250), 5% (mass/volume) bovine serum albumin (Sigma Aldrich, A3311), 0.5% (mass/volume) bovine serum albumin (Sigma Aldrich, A3311), 0.5% (mass/volume) TWEEN21 (Sigma Aldrich, P2287-100ML). Fixed cells were washed with IBS three times and permeabilized for 2 hours with IBS at room temperature. Cells were then stained with anti-ZO-1 Alexa Fluor 488 (to study the tight junctions) and Phalloidin Alexa Fluor 555 (to visualize cell boundaries by targeting actin filaments). These stains were incubated at room temperature for 1 hour. Additionally, nuclei were stained with Hoechst 33342 dye for 15 minutes at room temperature. After staining, cells were washed with DPBS and mounted onto slides. All images were captured using a Zeiss Axio Scan Z1 slide scanner. Z-stacks were acquired over 50 μm along the z-direction with 1.5 μm steps, and maximum intensity projections were used for all analyses.

Scanning electron microscopy: The study performed protocol for SEM imaging of RPE as published in [84]. In short, RPE was fixed in 4% paraformaldehyde (Electron Microscopy Science, 157-4-100) for 12 min, immediately following an experiment. The sample was then rinsed three times, over the course of 15 min, with PBS at room temperature and stored at 4° C. EM fixative (2.5% glutaraldehyde Grade 1 (Sigma Aldrich) and 10 mM CaCl₂)) was added to HEPES buffer, and the cells were incubated overnight. Samples were mounted on conductive carbon adhesive stubs and imaged using SEM (S-4800 Hitachi electron microscope).

Mathematical modeling. Using the equations for the impedance of resistors and capacitors and combining them using the same rules for resistors, the impedance of the equivalent circuit in FIG. 5C can be expressed as Equation 17.

$${Z}_{12} ( ω ) = {R}_{s o l A} + {R}_{s o l B} + \frac{{R}_{1}}{1 + {τ}_{1} ω i} + \frac{{R}_{2}}{1 + {τ}_{2} ω i} \qquad ( Eq . 17 )$$

For all measurement frequencies (a), the magnitude and phase shift for both circuits shown in FIG. 5C and one not having a shunt resister were maintained to be identical; thus, Z_{abs}(ω)=Z₁₂(ω). In setting these two equations equal to each other, and cross-multiplying the denominators, two equivalent, 4^{th}-order polynomial equations (ω⁴) in the following form arises: α₁ω⁴+β₁ω³+γ₁ω²+δ₁ω₁+∈₁ω⁰α₂ω⁴+β₂ω³+γ₂ω²+δ₂ω¹+∈₂ω⁰. Given the rules for equivalent polynomials, the coefficients corresponding to each order term can be set equal to each other. The 4th order terms (α₁=α₂) yielded the result,

$${β}_{1} = {β}_{2} \to \frac{{C}_{1} {C}_{2}}{{C}_{1} + {C}_{2}} = \frac{{C}_{a} {C}_{b}}{{C}_{a} + {C}_{b}} ,$$

which states, in effect, that the TEC for both circuits should be identical. The 2^{nd}- and 1^{st}-order terms produced equations: γ₁=γ₂→0=C₁C₂R₁R₂RₐRₛ+C₁C₂R₁R₂R_{b}Rₛ−C₁CₐR₁R₂RₐR_{b}−C₂CₐR₁R₂RₐR_{b}−C₁C₂R₁R₂RₐR_{b}−C₂C_{b}R₁R₂RₐR_{b}−C₁CₐR₁R₂RₐRₛ−C₂CₐR₁R₂RₐRₛ−C₁C_{b}R₁R₂RₐRₛ−C₂C_{b}R₁R₂RₐRₛ+C₁CₐR₁RₐR_{b}Rₛ+C₂CₐR₂RₐR_{b}Rₛ+C₁C_{b}R₁RₐR_{b}Rₛ+C₂C_{b}R₂RₐR_{b}Rₛ−CₐC_{b}R₁RₐR_{b}Rₛ−CₐC_{b}R₂RₐR_{b}Rₛ and δ₁=δ₂→0=C₁R₁R₂Rₐ+C₂R₁R₂Rₐ+C₁R₁R₂R_{b}+C₂R₁R₂R_{b}+C₁R₁R₂Rₛ+C_{2c}R₁R₂Rₛ−C₁R₁RₐRₛ−C₂R₂RₐRₛ−C₁R₁R_{b}Rₛ−C₂R₂R_{b}Rₛ+CₐR₁RₐR_{b}+CₐR₂RₐR_{b}+C_{b}R₁RₐR_{b}+C_{b}R₂RₐR_{b}+CₐR₁RₐRₛ+CₐR₂RₐRₛ+C_{b}R₁R_{b}Rₛ+C_{b}R₂R_{b}Rₛ−CₐRₐR_{b}Rₛ−C_{b}RₐR_{b}Rₛ The 0th-order coefficients produce the relationship:

$${ϵ}_{1} = {ϵ}_{2} \to {R}_{1} + {R}_{2} = \frac{{R}_{s} ( {R}_{a} + {R}_{b} )}{{R}_{s} + {R}_{a} + {R}_{b}} ,$$

which states that the total tissue resistances of both circuits should be equal. The total tissue resistance was then calculated by measuring the distance between the intersection points on the x-axis of the Nyquist diagram. The equation above states that the total tissue capacitances must be directly equal; however, it is not obvious from the diagram how they are related. Considering that the Nyquist plot is a 3-dimensional plot with coordinates < [symbol] (Z), [symbol] (Z),ω>, the study integrated the real ( [symbol] ) part of the impedance equation with respect to the measurement frequency, ω, to provide the relationship:

$${\int }_{0}^{∞} ℜ ( {Z}_{12} ( ω ) - {R}_{s o l A} - {R}_{s o l B} ) d ω = \frac{π}{2} ( \frac{1}{{C}_{1}} + \frac{1}{{C}_{2}} )$$

Following the rules of equivalent capacitors, the TEC (Cₜ) was determined as:

$$\frac{1}{{C}_{t}} = \frac{2}{π} {\int }_{0}^{∞} ℜ ( {Z}_{a b s} ( ω ) - {R}_{s o l A} - {R}_{s o l B} ) d ω .$$

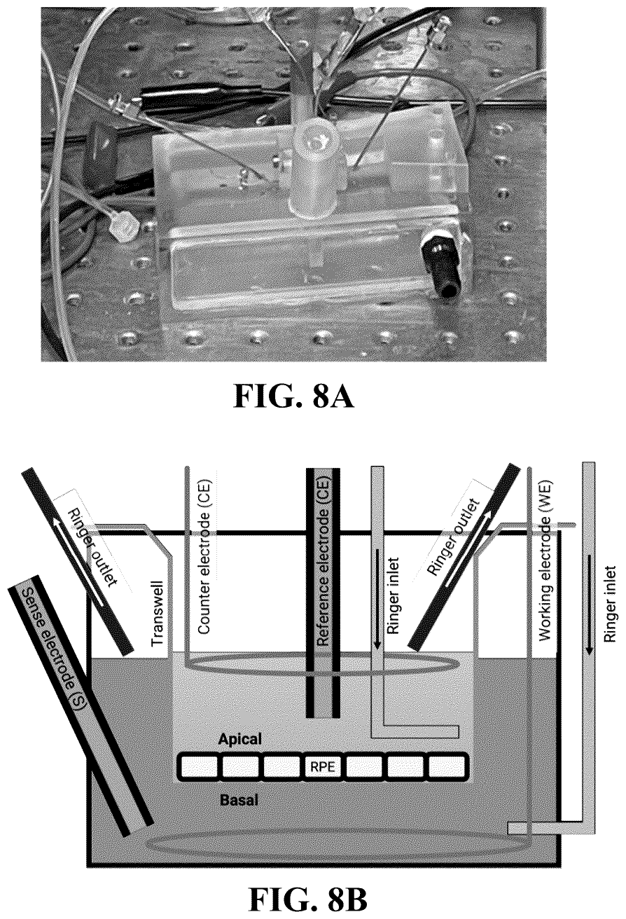

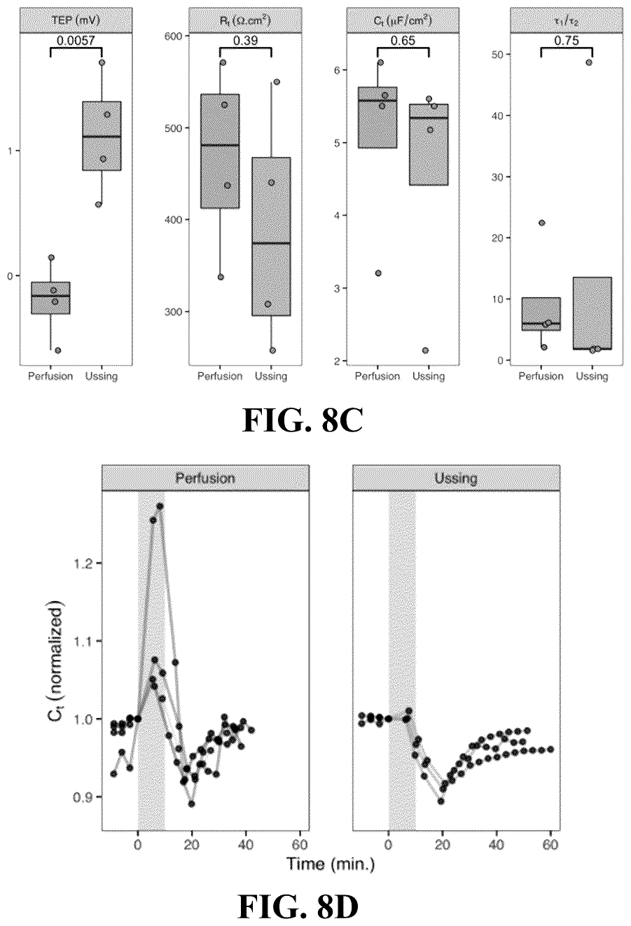

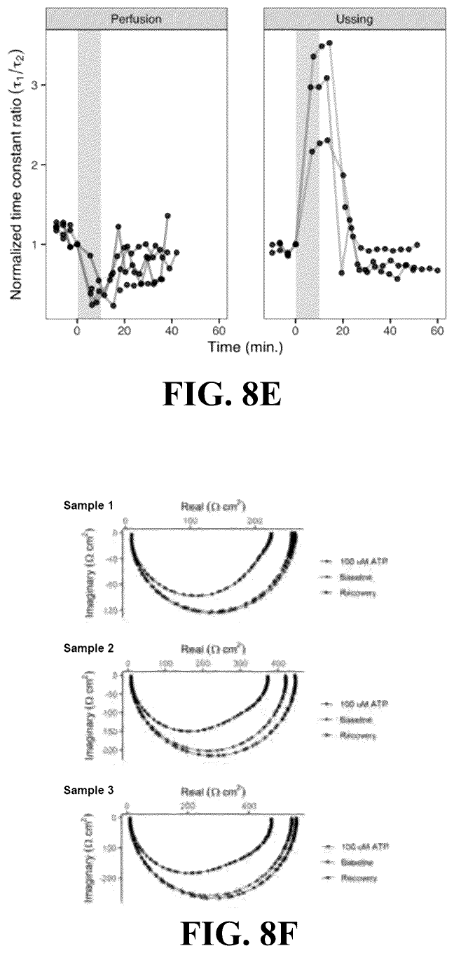

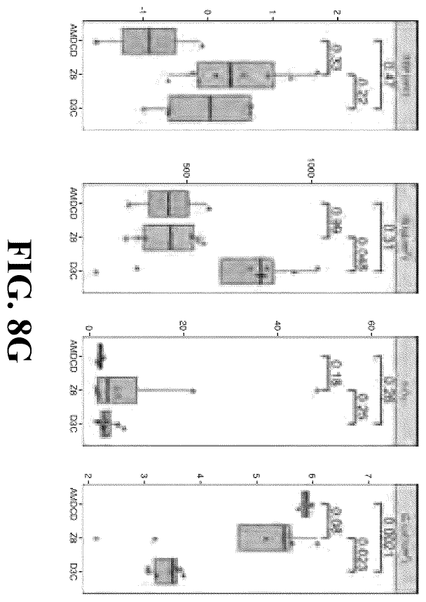

Chamber design and extracellular epithelial properties: A custom device was assembled to rapidly and noninvasively evaluate the evoked responses and recovery of RPE to extracellular ATP (FIG. 4.3). FIG. 8A shows a setup of the chamber employed in the study. FIG. 8B shows a schematic of the same.

The “perfusion” chamber in FIG. 8A was created using the same Acrylic material as the Ussing chamber but was designed to interface with an intact, 12-well Transwell insert (Corning costar 3460). Ag|AgCl wires were used as the working and counter electrodes. These electrodes were shaped into a symmetric ring of maximum diameter that fits inside the chamber and placed as far from the epithelia as possible to maximize uniformity of the current flux across the tissue. The sense and reference electrodes (used to measure TEP) were single-junction KCl reference electrodes. The KCl reference electrode was constructed with an Ag|AgCl wire that was inserted in a glass body (1.5 mm outer diameter) and backfilled with 3 M KCl. The terminus of these electrodes was made of a Vycor frit that enabled electrical contact between the 3 M KCl solution and the perfusate. Ag|AgCl electrodes were selected due to their rapid and reversible redox reactions, and the bridges were designed to minimize the effect of drifting Cl− ion concentrations on the electrode junction potential. The sense and reference electrodes were inserted through the cylinders extruding through the top of the device such that they were positioned as close as possible to the tissue. The design of the Acrylic cylinders included a kinematic constraint for the electrodes to ensure that the distance between the tissue and voltage-sensing probes was consistent across all experiments.

Custom chambers are commonly designed to execute unique or complex experiments when commercially available devices do not exist. Control experiments must be performed to verify the physiological mechanisms of epithelia in the new chamber are not significantly different than previous setups. For example, the magnitudes of extracellular tissue properties in normal Ringer's solution should be compared. Therefore, to demonstrate how tissue function can be validated using EIS, a single plate of iPSC-RPE (to minimize genetic variation) was split in half, and one half was tested in the Ussing chamber while the other half was tested in the perfusion chamber.

FIG. 8C shows the results of the perfusion versus Ussing chamber extracellular electrophysiological differences (n=4). The iPSC-RPE from a single cell line (Z8) was measured in both the traditional Ussing chamber and the custom perfusion chamber using EIS. Numbers above the bracket correspond to the p-value from a t-test between the two chambers. No statistical differences were observed between resistance, capacitance, and time constant ratio (Rₜ, Cₜ, and τ₁/τ₂, respectively) studied in the two chambers after normalizing for the cross-sectional area. However, there was a statistically significant difference be-tween the TEP for cells studied in the two chambers. TEP is sensitive to temperature, perfusion rate per chamber volume, and hydrostatic pressure during an experiment. For example, during Ussing chamber setup, adjusting the apical solution height dramatically affected epithelial TEP. Furthermore, solution temperatures in each chamber were measured to be different by up to 1.5° C., depending on the ambient temperature of the room. These effects have been observed in literature before, but the exact mechanisms are still poorly understood [70′], [71], [72′], [73′].

Furthermore, to ensure that the perfusion device can maintain the health and function of epithelia during long experiments, a simple experiment was devised to last at least 45 min; sufficient for rigorous RPE experiments performed in literature ([39′], [7′]). During this experiment the tissues were briefly exposed (approximately 10 min) to 100 μM ATP at the apical membrane. The time series data for all experiments were normalized: (1) if the cells were in an “unhealthy” physiological state, tissue properties will drift towards 0 (unstable), regardless of staring magnitude. (2) The relative magnitude and di-rection (positive or negative) of the response evoked by ATP, should be similar for both chambers. Specifically, if cell function is altered by chamber design, the magnitude and direction of the evoked response in the new chamber will likely appear different because the mechanisms involved in RPE ATP response require numerous channels to be expressed and functioning normally ([39′], [7′]).

It was observed that for both the perfusion and Ussing chambers, the epithelia responded with a similar magnitude and direction after ATP exposure. Furthermore, after ATP, the epithelia in both chambers returned to within 95% of baseline within 40 min. Most importantly, in neither chamber did the cell line start to lose resistance after up to an hour; a critical indicator of cell death. Furthermore, the TEP of the cells in both chambers was similar.

The data indicate that cell function does not appear to be impacted by chamber choice for long-duration experiments. Conventionally, if the chamber is (1) maintained from 32° C. to 37° C., (2) the perfusate is bubbled with sufficient oxygen (approximately 10% O₂ for RPE), and (3) the flow rate is fast enough to minimize the un-stirred layer and osmotic effects due to evaporation yet slow enough to avoid large shearing forces on the epithelia (from 2 mL/min to 5 mL/min), it appears that cell function remains stable over long-duration experiments. Finally, the magnitudes of the extracellular tissue properties need to be compared to ensure cell function did not appear to be impacted, in some statistically relevant manner, due to chamber configuration or design.

The previous data encapsulate the breadth of control experiments traditionally per-formed to validate the health of epithelia in a new chamber. However, using EIS, extra tissue parameters could be evaluated. For example, the tissue capacitance (Ct) can be determined.

FIG. 8D shows all normalized tissue capacitance (Ct) responses of iPSC-RPE (Z8) to 100 μM apical ATP exposure. Time 0 min corresponds to the moment of ATP exposure. The average duration of ATP exposure is shown by the grey box (approximately 10 min). FIG. 8D shows an interesting divergence between the direction and magnitude that the epithelia respond to apical ATP. First, the perfusion chamber capacitance appears to be noisier than the Ussing chamber; however, this can be explained by the much larger cross-sectional area of the perfusion chamber. The perfusion chamber has a cross-section of 1.12 cm²′ while the Ussing chamber has a cross-sectional area of 0.114 cm². With a smaller cross-sectional area, the tissue has a larger voltage response to the clamped current flux (35 μA/cm² for the Ussing chamber and 9 μA/cm² for the perfusion chamber). The maximum current for the perfusion chamber could not be clamped any higher without the addition of new stability and clamping errors due to specific hardware limitations. Thus, the lower current flux in the perfusion chamber likely contributed to the larger variation between measurements.

The second phenomenon is the notably different capacitance response magnitude and direction to apical ATP. In particular, the tissue capacitance increased during apical ATP exposure by an average of 11%, while it decreased in the Ussing chamber by an average 3%. The perfusion chamber's larger total volume—while utilizing an identical perfusion rate of 4.5 mL/min—resulted in a slower replacement of chamber volume per unit time. It was observed that stabilization after perturbations in extracellular solution was much slower for the perfusion chamber. Therefore, it was likely that the concentration of ATP took longer to reach 100 μM. It is possible that the RPE has additional mechanisms that respond to lower concentrations or that are attenuated when ATP is too high. Further evidence for this hypothesis stems from the magnitude and time course of the epithelial recovery after removing apical ATP. Specifically, in both the perfusion and Ussing chambers, the capacitance dropped for about 20 min before returning to baseline at nearly identical rates. In other words, the positive deflection in tissue capacitance—observed exclusively in the perfusion chamber during apical ATP—did not affect the magnitude and direction of the recovery after a 10-minute exposure.

Similarly, the extracellular time constant ratio (τ₁/τ₂)—only possible to obtain using EIS—yields another surprising result. FIG. 8E shows all normalized extracellular time constant ratios (τ1/τ2) for iPSC-RPE (Z8) response to 100 μM apical ATP exposure. Time 0 min corresponds to the moment of ATP exposure. The average duration of ATP exposure is shown by the grey box (approximately 10 min). The time constant ratio approximates the membrane-specific ratio of time constants for the apical and basal membranes (τₐ/τ_{b}). FIG. 8E indicates distinctly different membrane responses during ATP exposure between the two chambers despite a lack of statistical difference in baseline value. However, during recovery after ATP exposure, both chambers returned to their starting values, suggesting the inverse time constant ratio responses are reversible.

Little is known about how to interpret the tissue capacitance and the ratio of time constants response of RPE to ATP exposure. However, without the addition of these additional parameters, no significant difference in epithelial response to apical ATP can be detected. Thus, it appears that EIS can be used to reveal unique and previously unobservable phenomena in epithelia, such as the possible dosage-dependent response of iPSC-RPE to apical ATP.

Extracellular EIS as an analog for intracellular properties: There is a potential correlation between membrane time constants (tau ratio) and the—previously unobservable—membrane-specific voltage divider ratio a. To evaluate, epithelia that were recorded using traditional intracellular methods were also recorded with extracellular EIS. An example of Nyquist diagram changes observed during extracellular application of apical ATP is shown in FIG. 8F, which shows example Nyquist diagrams of iPSC-RPE (Z8) before, during, and after ATP exposure. Baseline condition corresponds to the impedance measurement immediately before apical ATP, 100 uM ATP corresponds to the impedance measurement during apical application of ATP (t≈6 min), and recovery corresponds to the impedance measurement after ATP washout (t≈30 min). Points on the plot correspond to measured data and the lines correspond to the best fit from the EIS circuit model.

For all 3 samples tested in FIG. 8F, a notable change in the shape of the Nyquist diagram during ATP compared to the baseline was detected. Furthermore, the baseline and recovery Nyquist diagrams appear to be nearly identical for all samples, indicating that the application of apical ATP was reversible. Sample 1 had an inverse apical membrane response compared to samples 2 and 3. Instead of increasing during apical ATP—as expected for healthy RPE—apical resistance decreased. When looking at FIG. 8F, a concomitant variation appears in the overall shape of the Nyquist plot for sample 1 compared to samples 2 and 3. Therefore, the voltage divider ratio (a=Rₐ/R_{b}) was compared quantitatively to the following extracellular EIS properties: time constant ratio (τ₁/τ₂), total tissue capacitance (Rₜ), and total tissue resistance (Rₜ).

The proportional decrease in tissue resistance and tissue capacitance relative to baseline during ATP exposure was approximately equal amongst all plots despite sample 1 starting at lower baseline values (samples 1, 2, and 3 are a subset of data included in FIG. 8D normalized Ussing chamber traces). Interestingly, the voltage divider ratio and time constant ratio have nearly identical magnitudes during baseline readings. In fact, the exact magnitude of all time constant and voltage divider ratios were nearly identical—per sample—during baseline measurements before apical ATP. However, during ATP exposure, sample 1 had a time-constant ratio deflection that was nearly double samples 2 and 3. Furthermore, all-time ratio constants increased in magnitude despite the direction in the voltage divider ratio changed. This is a limitation of extracellular EIS measurements because, without an intracellular electrode to identify the polarity of the time constant rations (namely, should the time constant be τ₁/τ₂ or τ₂/τ₁), the experimenter must predetermine—in the fitting algorithm—if the resultant time constant ratio is greater than or less than 1. Despite this limitation, the time constant ratio seems to quantitatively measure a difference between sample 1 and samples 2 and 3. In future work, the magnitude of this evoked response can be explored in greater detail. In particular, intracellular techniques can be leveraged to examine which membrane properties most significantly contribute to the magnitude of the time-constant ratio changes. Perhaps, providing scientists with a measurement capable of distinguishing membrane-specific variation between epithelia non-invasively.

Exploring the potential links between cell morphology and capacitance During cell culture, sensitive yet rapid and non-invasive techniques can help to assess cell line variability, in-line or at-line. In particular, iPSCs still suffer from notable variations during culture, which seem to be unrelated to cell line, seeding date, passage, or a number of other starting points. Thus, for iPSCs to become a standardized therapy and drug discovery platform, it would be tremendously beneficial for scientists to have quantitative metrics that are proven to be sensitive to cell line-specific properties.

Since electrophysiological parameters seem to lend useful insights into cell line-specific deviations (e.g., sample 3 vs. sample 1 and sample 2), EIS was performed across 3 donor iPSC-RPE cell lines (named AMDCD, Z8, and D3C) to investigate how consistent these extracellular electrophysiology terms are, per culture, and if there was any distinct clustering between cell lines.

From FIG. 8G, it seems that the strongest clustering per cell line—if any—is tissue capacitance. Capacitance is a function of the membrane dielectric constant F, the membrane surface area A, and the membrane thickness d. These terms describe the composition and the structure of the cell membrane, and are traditionally difficult to quantify.

### DISCUSSION

Epithelial cells form a barrier that plays a crucial role in the selective transport of ions, nutrients, and waste products throughout the body [1]-[3]. Damage or degeneration of these barrier-type tissues is often associated with common pathological conditions, such as celiac disease [4], cystic fibrosis [5], diabetes 6[6], and age-related macular degeneration (7), which collectively impact millions of people worldwide [8]-[12]]. Various techniques have been used to study the pathologies of these diseases in epithelial tissues, including morphology [13], protein biomarkers [14], gene expression [15], and electrophysiology [7]. Among these techniques, electrophysiology stands out as one of the few techniques that can provide insight into epithelial barrier function by measuring the electrical properties associated with the transport of critical ions, nutrients, and waste products in an environment similar to in vivo conditions [7],[16]. The most common measurement of epithelial electrophysiology is transepithelial resistance (TER), which is generally used to assess the formation of a barrier between epithelial cells by tight junctions [7],[17]-[27]. Additionally, some groups use electrochemical impedance spectroscopy (EIS) to quantify the transepithelial capacitance (TEC), which is parallel to the TER and may be proportional to morphological characteristics such as microvilli formation and basolateral infoldings [23],[27]. The TER and TEC are typically measured in commercially available devices such as an Ussing chamber [28], EndOhm [29], or STX2 [29] by placing electrodes on either side of the tissue layer in the extracellular space (e.g., at nodes 1 and 2 of FIG. 1A) and measuring the electrical response of the tissue to a stimulus [30]-[32].

The exemplary transepithelial measurements combine the apical, basolateral, and shunt (paracellular) transport pathways of epithelial tissues (FIG. 1A) into a single resistor or a simple parallel resistor and capacitor circuit [25], [33]-[35]. However, each pathway can have unique permeabilities to ions, making it necessary to independently characterize the electrical properties of each pathway using the intracellular circuit model, especially in disease, drug, and transport studies [30], [30]-[32],[38]-[42].

Traditional electrophysiological methods, which often use a single sharp pipette inserted into the cell (e.g., node 3) as described in relation to FIG. 1A can enable continuous measurements of apical or basolateral membrane voltages. These voltages can then be leveraged to estimate relative changes between the apical and basolateral transport pathways [38], [39]. However, these traditional approaches still cannot measure the resistances or capacitances of apical, basolateral, and shunt pathways without relying on additional assumptions, e.g., that the shunt resistance (Rₛ) remains constant, is practically infinite, or maintains a fixed ratio with the sum of the cell membrane resistances (Rₐ+R_{b}) [29], [41]-[45]. Such assumptions can obscure the underlying mechanisms, be they isolated increases in apical channel permeability, a combined effect involving both apical and basolateral channels, or changes restricted to basolateral channels, making it challenging to decipher the exact alterations in epithelial transport pathways.

Additional Discussion. Today, many scientists continue to rely on extracellular measurements to track the evolution of electrophysiological properties despite the additional, useful data that intracellular measurements provide. In particular, extracellular measurements are selected due to their simplicity, reduced cost, and significant throughput improvements. Furthermore, the non-invasive nature of extracellular experiments enables the return of Transwells back to culture after an experiment; assuming sufficient sterility.

The ability to measure the evoked responses and recovery of epithelia due to extracellular perturbations (e.g., 100 μM ATP) provides extra insight into cell function and health [39′]. Unfortunately, commercially available devices do not provide the hardware connections for simultaneous perfusion and electrophysiological assays (e.g., WPI's Endohm and Ussing chamber devices). In the instant study, a custom device was assembled to facilitate rapid and non-invasive extracellular analysis of RPE with continuous perfusion.

Scientists have turned to EIS to maintain the throughput advantages of extracellular experiments while attempting to maximize the amount of information collected during an experiment [66, 62, 75, 76, 77]. For example, Schifferdecker and Fromter [62] considered that the apical and basal membranes of confluent epithelia are electrically separated via the strong connections they make with neighboring cells (e.g., with the tight junctions). Consequently, both membranes have the ability to uniquely transfer and store charge and, thus, should behave as distinct low-pass filters, in series. Based on this theory, Schifferdecker and Fromter developed a mathematical model for epithelia that included two, parallel resistance and capacitance circuits in series. In their model, the distinction between the transcellular (through the cell) and shunt pathways (around and between the cells) employed in the exemplary method is lost; however, the equivalent filtering performed by the two membranes is preserved.

With this model, epithelia can be evaluated using an equivalent circuit that is more detailed than the extracellular equivalent circuit measured with the Ussing chamber, but not quite as detailed as the equivalent circuit measured with the intracellular technique. In Schifferdecker and Fromter's paper, they explored the implications of this simplification using the Nyquist diagram (also referred to as a Cole-Cole plot).

To generate the data visualized in a Nyquist diagram, the tissue can be measured at many distinct frequencies by clamping an alternating current across the epithelia. At each frequency, the amplitude and phase of the resultant voltage is measured and the relationship between input and output signal is represented by the complex impedance (Z= [symbol] +i [symbol] ). The magnitude of the real and imaginary components at each distinct frequency can be plot-ted as a discrete point in a graph where the x-axis is the real component ( [symbol] ), and the y-axis is the imaginary component ( [symbol] ). A collection of these points—each a distinct frequency—is the Nyquist diagram.

This relationship can be visualized in a variety of other, standard diagrams (e.g., Bode diagrams or Lissajous figures), but due to the specific properties of the epithelia, the Nyquist diagram is particularly powerful. For example, the asymptotes of the circuits in FIG. 4.1, as the measurement frequency approaches either 0 or ∞, will always approach the x-axis at two distinct locations. The distance between these two asymptotes is directly equal to the tissue resistance. Furthermore, epithelia are submerged in aqueous solutions that—themselves—have a small amount of resistance. Normally, this solution resistance is calculated before an experiment and subtracted from subsequent data, but because the solution's resistance is in series with the two membranes and has negligible capacitance, it appears as a small shift away from (0, 0) on the x-axis in a Nyquist diagram. In other words, the distance between the origin and the first intersection of the real axis is exactly equal to the solution resistance and eliminates the need to calculate this property before an experiment.

Schifferdecker and Fromter explored other graphical relationships between circuit properties and the Nyquist diagram, e.g., how a change in one of the two low pass filters' time constants (τ) would influence the shape of the corresponding Nyquist diagram. Despite the investigations that Schifferdecker and Fromter began, scientists have primarily focused on finding simple, independent measurements or assumptions about epithelia that enable the direct solution of when measured using extracellular EIS [5′]. This is likely because the exact magnitudes of the resistances and capacitances in the extracellular EIS circuit are not correlated—in any way—to the membrane-specific properties of elements.

Kreindler et al., for example, measured the shunt resistance pathway in a separate experiment (prior to EIS) by extrapolating the y-intercept of a tissue conductance versus short circuit current plot and assuming that the paracellular resistance remains constant throughout the experiment [74′]. Additionally, the instant study assumed that the paracellular resistance always contributes 71% of the net tissue conductance (Gtₜ=1/Rₜ) in a CF study of lung epithelia. These assumptions and measurements are critical for completely solving, but are some combination of (1) destructive to the cells, (2) need to be reverified for each, unique cell line and experiment protocol, and (3) potentially miss interesting cell behavior because it is necessarily assumed deviation from the normal function will not occur.

The assumption made by Kriendler et al. and Cottrill et al, for instance, would have obfuscated (potentially even misled) the interpretation of what happened with the epithelia. Generally, the shunt resistance was neither a constant nor was it a fixed proportion of total tissue resistance. In fact, in all three RPE recordings, the shunt resistance both (1) changed during the application of ATP and (2) the magnitude of the change was not proportional to the total tissue resistance.

This error is a major issue with conventional techniques. Instead of trying to solve for the complete circuit using extracellular EIS and potentially erroneous or ill-posed assumptions, the instant study tried to instead observe if changes in time constants can be used as a crude analog of voltage divider ratio (a) to provide a high throughput and simple-to-perform technique to bridge the gap between extracellular and intracellular measurements, e.g., for drug screening. An experimenter could use the crude bridge to quickly identify compounds or interactions that generate membrane-specific responses that should be followed up with the more comprehensive (and accurate) intracellular and EIS combination.

At best, extracellular EIS has provided scientists with an additional transepithelial capacitance (TEC) term that, to date, lacks a rigorous link to real cell properties. It is likely that cell capacitance is linked to cell morphology; however, which morphological parameters are most strongly correlated to capacitance needs further investigation (e.g., cell cross-sectional area, membrane surface area, apical process density, length, and type, as well as basal membrane folding). Equations for capacitance indicate that capacitance can be used as an analog for cell surface area (given membrane thickness and dielectric constant do not change from experiment to experiment); however, experimental data are lacking to validate this claim [78, 74, 79, 72, 64, 80, 67, 81, 66, 62, 71, 69, 82].

In contrast, the exemplary system and method, e.g., as described in relation to FIGS. 1A and 1B, can quantify the membrane-specific properties that make up an equivalent electrical circuit of intact epithelial tissues, using a combination of an intracellular pipette and extracellular EIS to resolve the transcellular and paracellular electrical properties without constraint-based assumptions previously required to evaluate these metrics [35″, 41″], [43″][45″], [57″]. Specifically, the exemplary system and method (e.g., FIGS. 1A, 1B) can measure the apical resistance Rₐ, apical capacitance Cₐ, basolateral resistance R_{b}, basolateral capacitance C_{b}, and shunt resistance Rₛ of intact epithelial tissues. Furthermore, the exemplary system and method (e.g., FIGS. 1A, 1B) can simultaneously measure the resistance of the surrounding media and culture substrate to compensate for changes throughout an experiment, further improving the accuracy of the epithelial parameters. Rigorous validation with electronic cell components quantifies the accuracy of the exemplary system and method (e.g., FIGS. 1A, 1B) and an examination of typical biological variability suggests that the model is suitable for use in studying epithelia.

The addition of exemplary system and method (e.g., FIGS. 1A, 1B) in existing 3 P-Classical setups can enable a nuanced understanding of epithelial physiology based on additional electrical parameters to enhance epithelial transport dynamics quantification. These parameters previously required additional measurements or assumptions to evaluate.

For research, the exemplary system and method (e.g., FIGS. 1A, 1B) can provide a more precise readout of epithelial physiology and enhance the details extracted when studying novel pathways diseases. It also, potentially, provides dynamic readouts of cell morphology without a microscope or other destructive techniques. The utility of the exemplary system and method (e.g., FIGS. 1A, 1B) extend beyond basic research. In the clinical realm, this method offers a new avenue for drug testing where membrane-specific changes or interactions are underdetermined when using 3 P-Classical or in assumption-based methods that assume normal cell function. Furthermore, this method could be used as a higher sensitivity quality control metric in transplantation therapies.

### CONCLUSION

In this specification and in the claims that follow, reference will be made to a number of terms, which shall be defined to have the following meanings:

Throughout the description and claims of this specification, the word “comprise” and other forms of the word, such as “comprising” and “comprises,” means including but not limited to, and are not intended to exclude, for example, other additives, segments, integers, or steps. Furthermore, it is to be understood that the terms comprise, comprising, and comprises as they relate to various aspects, elements, and features of the disclosed invention also include the more limited aspects of “consisting essentially of” and “consisting of”

Ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It should be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

For the terms “for example” and “such as,” and grammatical equivalences thereof, the phrase “and without limitation” is understood to follow unless explicitly stated otherwise.

The following patents, applications and publications as listed below and throughout this document are hereby incorporated by reference in their entirety herein.

### REFERENCE LIST #1

[1] Schumann, M., Siegmund, B., Schulzke, J. D. & Fromm, M. Celiac Disease: Role of the Epithelial Barrier. Cell. Mol. Gastroenterol. Hepatol. 3, 150-162 (2017).
[2] Skach, W. R. Cystic Fibrosis Methods and Protocols. Cystic Fibrosis Methods and Protocols (2002). doi:10.1385/1592591876.
[3] Tang, S. C. W., Leung, J. C. K., Chan, L. Y. Y., Tsang, A. W. L. & Lai, K. N. Activation of tubular epithelial cells in diabetic nephropathy and the role of the peroxisome proliferator-activated receptor-γ agonist. J. Am. Soc. Nephrol. 17, 1633-1643 (2006).
[4] Miyagishima, K. J. et al. In Pursuit of Authenticity: Induced Pluripotent Stem Cell-Derived Retinal Pigment Epithelium for Clinical Applications. Stem Cells Transl. Med. 5, 1562-1574 (2016).
[5] Anderson, J. M. & Itallie, C. M. V. Physiology and function of the tight junction. Cold Spring Harb. Perspect. Biol. 1, a002584 (2009).
[6] Rizzolo, L. J., Peng, S., Luo, Y. & Xiao, W. Integration of tight junctions and claudins with the barrier functions of the retinal pigment epithelium. Prog. Retin. Eye Res. 30, 296-323 (2011).
[7] Cereijido, M., Robbins, E. S., Dolan, W. J., Rotunno, C. A. & Sabatini, D. D. Polarized monolayers formed by epithelial cells on a permeable and translucent support. J. Cell Biol. 77, 853-880 (1978).
[8] Farhadifar, R., Röper, J.-C., Aigouy, B., Eaton, S. & Jülicher, F. The Influence of Cell Mechanics, Cell-Cell Interactions, and Proliferation on Epithelial Packing. Curr. Biol. 17, 2095-2104 (2007).
[9] Jin Lee, H., W. Wark, A. & M. Corn, R. Microarray methods for protein biomarker detection. Analyst 133, 975-983 (2008).
[10] Lovén, J. et al. Revisiting Global Gene Expression Analysis. Cell 151, 476-482 (2012).
[11] Hassan, Q., Ahmadi, S. & Kerman, K. Recent advances in monitoring cell behavior using cell-based impedance spectroscopy. Micromachines 11, (2020).
[12] Dorjsuren, D. et al. A platform of assays for the discovery of anti-Zika small-molecules with activity in a 3D-bioprinted outer-blood-retina model. PLOS ONE 17, e0261821 (2022).
[13] Zoio, P., Lopes-Ventura, S. & Oliva, A. Barrier-on-a-Chip with a Modular Architecture and Integrated Sensors for Real-Time Measurement of Biological Barrier Function.

Micromachines 12, 816 (2021).
[14] Vigh, J. P. et al. Transendothelial Electrical Resistance Measurement across the Blood-Brain Barrier: A Critical Review of Methods. Micromachines 12, 685 (2021).
[15] Raut, B., Chen, L.-J., Hori, T. & Kaji, H. An Open-Source Add-On EVOM® Device for Real-Time Transepithelial/Endothelial Electrical Resistance Measurements in Multiple Transwell Samples. Micromachines 12, 282 (2021).
[16] Rajabzadeh, M. et al. A PCB-Based 24-Ch. MEA-EIS Allowing Fast Measurement of TEER. IEEE Sens. J. 21, 13048-13059 (2021).
[17] Schaub, N. J. et al. Deep learning predicts function of live retinal pigment epithelium from quantitative microscopy. J. Clin. Invest. 130, 1010-1023 (2020).
[18] Linz, G. et al. Cell barrier characterization in transwell inserts by electrical impedance spectroscopy. Biosens. Bioelectron. 165, 112345 (2020).
[19] Zhao, P. Y. et al. TRP channels localize to subdomains of the apical plasma membrane in human fetal retinal pigment epithelium. Invest. Ophthalmol. Vis. Sci. 56, 1916-1923 (2015).
[20] Srinivasan, B. et al. TEER Measurement Techniques for In Vitro Barrier Model Systems. J. Lab. Autom. 20, 107-126 (2015).
[21] Schmid, T., Bogdan, M. & Günzel, D. Discerning Apical and Basolateral Properties of HT-29/B6 and IPEC-J2 Cell Layers by Impedance Spectroscopy, Mathematical Modeling and Machine Learning. PLoS ONE 8, 1-12 (2013).
[22] Onnela, N. et al. Electric impedance of human embryonic stem cell-derived retinal pigment epithelium. Med. Biol. Eng. Comput. 50, 107-116 (2012).
[23] Yeste, J., Illa, X., Alvarez, M. & Villa, R. Engineering and monitoring cellular barrier models. J. Biol. Eng. 12, 18 (2018).
[24] Fromm, M., Schulzke, J. D. & Hegel, U. Epithelial and subepithelial contributions to transmural electrical resistance of intact rat jejunum, in vitro. Pflüg. Arch. Eur. J. Physiol. 405, 400-402 (1985).
[25] Schifferdecker, E. & Frömter, E. The AC impedance of necturus gallbladder epithelium. Pflüg. Arch. Eur. J. Physiol. 377, 125-133 (1978).
[26] Cottrill, K. A. et al. Sphingomyelinase decreases transepithelial anion secretion in airway epithelial cells in part by inhibiting CFTR-mediated apical conductance. Physiol. Rep. 9, 1-17 (2021).
[27] Awayda, M. S., Driessche, W. V. & Helman, S. I. Frequency-dependent capacitance of the apical membrane of frog skin: Dielectric relaxation processes. Biophys. J. 76, 219-232 (1999).
[28] Lim, J. J., Kottra, G., Kampmann, L. & Fromter, E. Chapter 2 Impedance Analysis of Necturus Gallbladder Epithelium Using Extra- and Intracellular Microelectrodes. in Current Topics in Membranes and Transport (eds. Kleinzeller, A. & Bronner, F.) vol. 20 27-46 (Academic Press, 1984).
[29] Sharma, R. et al. Epithelial phenotype restoring drugs suppress macular degeneration phenotypes in an iPSC model. Nat. Commun. 12, 7293 (2021).
[30] Sharma, R., Bose, D., Montford, J., Ortolan, D. & Bharti, K. Triphasic developmentally guided protocol to generate retinal pigment epithelium from induced pluripotent stem cells. STAR Protoc. 3, 101582 (2022).
[31] Sharma, R. et al. Clinical-grade stem cell-derived retinal pigment epithelium patch rescues retinal degeneration in rodents and pigs. Sci. Transl. Med. 11, eaat5580 (2019).
[32] Lewallen, C. F. et al. High-yield, automated intracellular electrophysiology in retinal pigment epithelia. J. Neurosci. Methods 328, 108442 (2019).
[33] Peterson, W. M., Meggyesy, C., Yu, K. & Miller, S. S. Extracellular ATP activates calcium signaling, ion, and fluid transport in retinal pigment epithelium. J. Neurosci. 17, 2324-2337 (1997).
[34] Maminishkis, A. et al. The P2Y2 Receptor Agonist INS37217 Stimulates RPE Fluid Transport In Vitro and Retinal Reattachment in Rat. Invest. Ophthalmol. Vis. Sci. 43, 3555-3566 (2002).
[35] Frömter, E. Electrophysiological analysis of rat renal sugar and amino acid transport. I. Basic phenomena. Pflugers Arch. 393, 179-189 (1982).
[36] Frömter, E. & Gebler, B. Electrical properties of amphibian urinary bladder epithelia. Pflüg. Arch. 371, 99-108 (1977).
[37] Blaug, S. et al. ENaC- and CFTR-dependent ion and fluid transport in mammary epithelia. Am. J. Physiol.-Cell Physiol. 281, C633-C648 (2001).
[38] Wills, N. K., Lewis, S. A. & Eaton, D. C. Active and passive properties of rabbit descending colon: A microelectrode and nystatin study. J. Membr. Biol. 45, 81-108 (1979).
[39] Kottra, G., Haase, W. & Fromter, E. Tight-junction tightness of Necturus gall bladder epithelium is not regulated by cAMP or intracellular Cₐ2+. II Impedance measurements. Pflugers Arch. 425, 535-545 (1993).
[40] Bello-Reuss, E. Cell membranes and paracellular resistances in isolated renal proximal tubules from rabbit and Ambystoma. J. Physiol. 370, 25-38 (1986).
[41] Kottra G, Fromter E. Rapid determination of intraepithelial resistance barriers by alternating current spectroscopy. II. Test of model circuits and quantification of results. Pflugers Arch 402: 421-432, 1984. doi:10.1007/bf00583943.
[42] Clausen C, Lewis S A, Diamond J M. Impedance analysis of a tight epithelium using a distributed resistance model. Biophys J 26: 291-317, 1979. doi:10.1016/S0006-3495(79)85250-9.
[43] Srinivasan B, Kolli A R, Esch M B, Abaci H E, Shuler M L, Hickman J J. TEER measurement techniques for in vitro barrier model systems. J Lab Autom 20: 107-126, 2015. doi:10.1177/2211068214561025.
[44] Sharma R, Bose D, Montford J, Ortolan D, Bharti K. Triphasic developmentally guided protocol to generate retinal pigment epithelium from induced pluripotent stem cells. STAR Protoc 3: 101582, 2022. doi:10.1016/j.xpro.2022.101582.
[45] Lewallen C F, Wan Q, Maminishkis A, Stoy W, Kolb I, Hotaling N, Bharti K, Forest C R. High-yield, automated intracellular electrophysiology in retinal pigment epithelia. J Neurosci Methods 328: 108442, 2019. doi:10.1016/j.jneumeth.2019.108442.
[46] Miyagishima K J, Wan Q, Corneo B, Sharma R, Lotfi M R, Boles N C, Hua F, Maminishkis A, Zhang C, Blenkinsop T, Khristov V, Jha B S, Memon O S, D'Souza S, Temple S, Miller S S, Bharti K. In pursuit of authenticity: induced pluripotent stem cell-derived retinal pigment epithelium for clinical applications. Stem cells Transl Med 5: 1562-1574, 2016. doi:10.5966/sctm.2016-0037.
[47] Schaub N J, Hotaling N A, Manescu P, Padi S, Wan Q, Sharma R, George A, Chalfoun J, Simon M, Ouladi M, Simon C G, Bajcsy P, Bharti K. Deep learning predicts function of live retinal pigment epithelium from quantitative microscopy. J Clin Invest 130: 1010-1023, 2020. doi:10.1172/JCI131187.
[48] Tran V, Zhang X, Cao L, Li H, Lee B, So M, Sun Y, Chen W, Zhao M. Synchronization modulation increases transepithelial potentials in MDCK monolayers through Na/K pumps. PLoS One 8: e61509, 2013. doi:10.1371/journal.pone.0061509.
[49] Blaug S, Rymer J, Jalickee S, Miller S S. P2 purinoceptors regulate calcium-activated chloride and fluid transport in 31EG4 mammary epithelia. Am J Physiol Cell Physiol 284: C897-C909, 2003. doi:10.1152/ajpcell.00238.2002.
[50] Schifferdecker E, Fromter E. The A C impedance of necturus gallbladder epithelium. Pflugers Arch 377: 125-133, 1978. doi:10.1007/BF00582842.

### REFERENCE LIST #2

[1′] Anderson J M, Itallie C M. Physiology and function of the tight junction. Cold Spring Harb Perspect Biol 1: a002584, 2009. doi:10.1101/cshperspect.a002584.
[2′] Rizzolo L J, Peng S, Luo Y, Xiao W. Integration of tight junctions and claudins with the barrier functions of the retinal pigment epithelium. Prog Retin Eye Res 30: 296-323, 2011. doi:10.1016/j.preteyeres.2011.06.002.
[3′] Cereijido M, Robbins E S, Dolan W J, Rotunno C A, Sabatini D D. Polarized monolayers formed by epithelial cells on a permeable and translucent support. J Cell Biol 77: 853-880, 1978. doi:10.1083/jcb.77.3.853.
[4′] Schumann M, Siegmund B, Schulzke J D, Fromm M. Celiac disease: role of the epithelial barrier. Cell Mol Gastroenterol Hepatol 3: 150-162, 2017. doi:10.1016/j.jcmgh.2016.12.006.
[5′] Skach W R (Editor). Cystic Fibrosis Methods and Protocols. Totowa, N J: Humana, 2002.
[6′] Tang S C, Leung J C, Chan L Y, Tsang A W, Lai K N. Activation of tubular epithelial cells in diabetic nephropathy and the role of the peroxisome proliferator-activated receptor-7 agonist. J Am Soc Nephrol 17: 1633-1643, 2006. doi:10.1681/ASN.2005101113.
[7′] Miyagishima K J, Wan Q, Corneo B, Sharma R, Lotfi M R, Boles N C, Hua F, Maminishkis A, Zhang C, Blenkinsop T, Khristov V, Jha B S, Memon O S, D'Souza S, Temple S, Miller S S, Bharti K. In pursuit of authenticity: induced pluripotent stem cell-derived retinal pigment epithelium for clinical applications. Stem cells Transl Med 5: 1562-1574, 2016. doi:10.5966/sctm.2016-0037.
[8′] Choung R S, Unalp-Arida A, Ruhl C E, Brantner T L, Everhart J E, Murray J A. Less hidden celiac disease but increased gluten avoidance without a diagnosis in the USA: findings from the National Health and Nutrition Examination Surveys from 2009 to 2014. Mayo Clin Proc 92: P30-P38, 2016. doi:10.1016/j.mayocp.2016.10.012..
[9′] Caio G, Volta U, Sapone A, Leffler D A, De Giorgio R, Catassi C, Fasano A. Celiac disease: a comprehensive current review. BMC Med 17: 142, 2019. doi:10.1186/s12916-019-1380-z.
[10′] Allan K M, Farrow N, Donnelley M, Jaffe A, Waters S A. Treatment of cystic fibrosis: from gene- to cell-based therapies. Front Pharmacol 12: 639475, 2021. doi:10.3389/fphar.2021.639475.
[11′] Reed J, Bain S, Kanamarlapudi V. A review of current trends with type 2 diabetes epidemiology, aetiology, pathogenesis, treatments and future perspectives. Diabetes Metab Syndr Obes 14: 3567-3602, 2021. doi:10.2147/DMSO.S319895.
[12′] Ammar M J, Hsu J, Chiang A, Ho A C, Regillo C D. Age-related macular degeneration therapy: a review. Curr Opin Ophthalmol 31: 215-221, 2020. doi:10.1097/ICU.0000000000000657.
[13′] Farhadifar R, Roper J C, Aigouy B, Eaton S, JUlicher F. The influence of cell mechanics, cell-cell interactions, and proliferation on epithelial packing. Curr Biol 17: 2095-2104, 2007. doi:10.1016/j.cub.2007.11.049.
[14′] Jin Lee H W, Wark A M, Corn R. Microarray methods for protein biomarker detection. Analyst 133: 975-983, 2008. doi:10.1039/B717527B.
[15′] Lovén J, Orlando D A, Sigova A A, Lin C Y, Rahl P B, Burge C B, Levens D L, Lee T I, Young R A. Revisiting global gene expression analysis. Cell 151: 476-482, 2012. doi:10.1016/j.cell.2012.10.012.
[16′] Hassan Q, Ahmadi S, Kerman K. Recent advances in monitoring cell behavior using cell-based impedance spectroscopy. Micromachines (Basel) 11: 590, 2020. doi:10.3390/MI11060590.
[17′] Dorjsuren D, Eastman R T, Song M J, Yasgar A, Chen Y, Bharti K, Zakharov A V, Jadhav A, Ferrer M, Shi P Y, Simeonov A. A platform of assays for the discovery of anti-Zika small-molecules with activity in a 3D-bioprinted outer-blood-retina model. PLoS One 17: e0261821, 2022. doi:10.1371/journal.pone.0261821.
[18′] Zoio P, Lopes-Ventura S, Oliva A. Barrier-on-a-chip with a modular architecture and integrated sensors for real-time measurement of biological barrier function. Micromachines 12: 816, 2021. doi:10.3390/mi12070816.
[19′] Vigh J P, Kincses A, OzgUr B, Walter F R, Santa-Maria A R, Valkai S, Vastag M, Neuhaus W, Brodin B, Dér A, Deli M A. Transendothelial electrical resistance measurement across the blood-brain barrier: a critical review of methods. Micromachines (Basel) 12: 685, 2021. doi:10.3390/mil2060685.
[20′] Raut B, Chen L J, Hori T, Kaji H. An open-source add-on EVOM® device for real-time transepithelial/endothelial electrical resistance measurements in multiple transwell samples. Micromachines (Basel) 12: 282, 2021. doi:10.3390/mil2030282.
[21′] Rajabzadeh M, Ungethum J, Herkle A, Schilpp C, Becker J, Fauler M, Wittekindt O, Frick M, Ortmanns M. A PCB-based 24-Ch. MEA-EIS allowing fast measurement of TEER. IEEE Sensors J 21: 13048-13059, 2021. doi:10.1109/JSEN.2021.3067823.
[22′] Schaub N J, Hotaling N A, Manescu P, Padi S, Wan Q, Sharma R, George A, Chalfoun J, Simon M, Ouladi M, Simon C G, Bajcsy P, Bharti K. Deep learning predicts function of live retinal pigment epithelium from quantitative microscopy. J Clin Invest 130: 1010-1023, 2020. doi:10.1172/JCI131187.
[23′] Linz G, Djeljadini S, Steinbeck L, Kose G, Kiessling F, Wessling M. Cell barrier characterization in transwell inserts by electrical impedance spectroscopy. Biosens Bioelectron 165: 112345, 2020. doi:10.1016/j.bios.2020.112345.
[24′] Zhao P Y, Gan G, Peng S, Wang S B, Chen B, Adelman R A, Rizzolo U. TRP channels localize to subdomains of the apical plasma membrane in human fetal retinal pigment epithelium. Invest Ophthalmol Vis Sci 56: 1916-1923, 2015. doi:10.1167/iovs.14-15738.
[25′] Srinivasan B, Kolli A R, Esch M B, Abaci H E, Shuler M L, Hickman J J. TEER measurement techniques for in vitro barrier model systems. J Lab Autom 20: 107-126, 2015. doi:10.1177/2211068214561025.
[26′] Schmid T, Bogdan M, GUnzel D. Discerning apical and basolateral properties of HT-29/B6 and IPEC-J2 cell layers by impedance spectroscopy, mathematical modeling and machine Learning. PLoS ONE 8: e62913-12, 2013. doi:10.1371/journal.pone.0062913.
[27′] Onnela N, Savolainen V, Juuti-Uusitalo K, Vaajasaari H, Skottman H, Hyttinen J. Electric impedance of human embryonic stem cell-derived retinal pigment epithelium. Med Biol Eng Comput 50: 107-116, 2012. doi:10.1007/s11517-011-0850-z.
[28′] Gondzik V, Awayda M S. Methods for stable recording of short-circuit current in a Na+-transporting epithelium. Am J Physiol Cell Physiol 301: C162-C170, 2011. doi:10.1152/ajpcell.00459.2010.
[29′] Sheller R A, Cuevas M E, Todd M C. Comparison of transepithelial resistance measurement techniques: chopsticks vs. endohm. Biol Proced Online 19: 4, 2017. doi:10.1186/s12575-017-0053-6.
[30′] Fromter E. The route of passive ion movement through the epithelium of Necturus gallbladder. J Membr Biol 8: 259-301, 1972. doi:10.1007/BF01868106.
[31′] Miller S S, Steinberg R H. Passive ionic properties of frog retinal pigment epithelium. J Membr Biol 36: 337-372, 1977. doi:10.1007/BF01868158.
[32′] Schifferdecker E, Fromter E. The A C impedance of necturus gallbladder epithelium. Pflugers Arch 377: 125-133, 1978. doi:10.1007/BF00582842.
[33′] Yeste J, Illa X, Alvarez M, Villa R. Engineering and monitoring cellular barrier models. J Biol Eng 12: 18, 2018. doi:10.1186/s13036-018-0108-5.
[34′] Fromm M, Schulzke J D, Hegel U. Epithelial and subepithelial contributions to transmural electrical resistance of intact rat jejunum, in vitro. Pflugers Arch 405: 400-402, 1985. doi:10.1007/BF00595695.
[35′] Reuss L, Finn A L. Passive electrical properties of toad urinary bladder epithelium: Intercellular electrical coupling and transepithelial cellular and shunt conductances. J Gen Physiol 64: 1-25, 1974. doi:10.1085/jgp.64.1.1.
[36′] Kottra G, Fromter E. Rapid determination of intraepithelial resistance barriers by alternating current spectroscopy. I I. Test of model circuits and quantification of results. Pflugers Arch 402: 421-432, 1984. doi:10.1007/bf00583943.
[37′] Clausen C, Lewis S A, Diamond J M. Impedance analysis of a tight epithelium using a distributed resistance model. Biophys J 26: 291-317, 1979. doi:10.1016/50006-3495(79)85250-9.
[38′] Maminishkis A, Jalickee S, Blaug S A, Rymer J, Yerxa B R, Peterson W M, Miller S S. The P2Y2 receptor agonist INS37217 stimulates RPE fluid transport in vitro and retinal reattachment in rat. Invest Ophthalmol Vis Sci 43: 3555-3566, 2002.
[39′] Peterson W M, Meggyesy C, Yu K, Miller S S. Extracellular ATP activates calcium signaling, ion, and fluid transport in retinal pigment epithelium. J Neurosci 17: 2324-2337, 1997. doi:10.1523/JNEUROSCI.17-07-02324.1997.
[40′] Bialek S, Miller S S. K+ and Cl− transport mechanisms in bovine pigment epithelium that could modulate subretinal space volume and composition. J Physiol 475: 401-417, 1994. doi:10.1113/jphysiol.1994.sp020081.
[41′] Cottrill K A, Peterson R J, Lewallen C F, Koval M, Bridges R J, McCarty N A. Sphingomyelinase decreases transepithelial anion secretion in airway epithelial cells in part by inhibiting CFTR-mediated apical conductance. Physiol Rep 9: e14928-17, 2021. doi:10.14814/phy2.14928.
[42′] Awayda M S, Driessche W V, Helman S I. Frequency-dependent capacitance of the apical membrane of frog skin: Dielectric relaxation processes. Biophys J 76: 219-232, 1999. doi:10.1016/50006-3495(99)77191-2.
[43′] Joseph D P, Miller S S. Apical and basal membrane ion transport mechanisms in bovine retinal pigment epithelium. J Physiol 435: 439-463, 1991. doi:10.1113/jphysiol.1991.sp018518.
[44′] Bello-Reuss E. Cell membranes and paracellular resistances in isolated renal proximal tubules from rabbit and Ambystoma. J Physiol 370: 25-38, 1986. doi:10.1113/jphysiol.1986.sp015920.
[45′] Krug S M, Fromm M, GUnzel D. Two-path impedance spectroscopy for measuring paracellular and transcellular epithelial resistance. Biophys J 97: 2202-2211, 2009. doi:10.1016/j.bpj.2009.08.003.
[46′] Lewallen C F, Wan Q, Maminishkis A, Stoy W, Kolb I, Hotaling N, Bharti K, Forest C R. High-yield, automated intracellular electrophysiology in retinal pigment epithelia. J Neurosci Methods 328: 108442, 2019. doi:10.1016/j.jneumeth.2019.108442.
[47′] Kodandaramaiah S B, Franzesi G T, Chow B Y, Boyden E S, Forest C R. Automated whole-cell patch-clamp electrophysiology of neurons in vivo. Nat Methods 9: 585-587, 2012. doi:10.1038/nmeth.1993.
[48′] Kolb I, Landry C R, Yip M C, Lewallen C F, Stoy W A, Lee J, Felouzis A, Yang B, Boyden E S, Rozell C J, Forest C R. PatcherBot: a single-cell electrophysiology robot for adherent cells and brain slices. J Neural Eng 16: 046003, 2019. doi:10.1088/1741-2552/abl834.
[49′] Stoy W A, Kolb I, Holst G L, Liew Y, Pala A, Yang B, Boyden E S, Stanley G B, Forest C R. Robotic navigation to subcortical neural tissue for intracellular electrophysiology in vivo. J Neurophysiol 118: 1141-1150, 2017. doi:10.1152/jn.00117.2017.
[50′] Holst G, Stoy W A, Yang B, Kolb I, Kodandaramaiah S B, Li L, Knoblich U, Zeng H, Haider B, Boyden E S, Forest C R. Autonomous patch clamp robot for functional characterization of neurons in vivo: development and application to mouse visual cortex. J Neurophysiol 121: 2341-2357, 2019. doi:10.1152/jn.00738.2018.
[51′] Kottra G, Haase W, Fromter E. Tight-junction tightness of Necturus gall bladder epithelium is not regulated by cAMP or intracellular Ca2+. II. Impedance measurements. Pflugers Arch 425: 535-545, 1993. doi:10.1007/BF00374881.
[52′] Ohki S. The electrical capacitance of phospholipid membranes. Biophys J 9: 1195-1205, 1969. doi:10.1016/S0006-3495(69)86445-3.
[53′] Gallemore R P, Hernandez E, Tayyanipour R, Fujii S, Steinberg R H. Basolateral membrane Cl− and K+ conductances of the dark-adapted chick retinal pigment epithelium. J Neurophysiol 70: 1656-1668, 1993. doi:10.1152/jn.1993.70.4.1656.
[54′] Sharma R, Bose D, Montford J, Ortolan D, Bharti K. Triphasic developmentally guided protocol to generate retinal pigment epithelium from induced pluripotent stem cells. STAR Protoc 3: 101582, 2022. doi:10.1016/j.xpro.2022.101582.
[55′] Miller S S, Edelman J L. Active ion transport pathways in the bovine retinal pigment epithelium. J Physiol 424: 283-300, 1990. doi:10.1113/jphysiol.1990.sp018067.
[56′] Lin H, Kenyon E, Miller S S. Na-dependent pHi regulatory mechanisms in native human retinal pigment epithelium. Invest Ophthalmol Vis Sci 33: 3528-3538, 1992.
[57′] Wills N K, Lewis S A, Eaton D C. Active and passive properties of rabbit descending colon: a microelectrode and nystatin study. J Membr Biol 45: 81-108, 1979. doi:10.1007/BF01869296.
[58′] Tran V, Zhang X, Cao L, Li H, Lee B, So M, Sun Y, Chen W, Zhao M. Synchronization modulation increases transepithelial potentials in MDCK monolayers through Na/K pumps. PLoS One 8: e61509, 2013. doi:10.1371/journal.pone.0061509.
[59′] Blaug S, Rymer J, Jalickee S, Miller S S. P2 purinoceptors regulate calcium-activated chloride and fluid transport in 31EG4 mammary epithelia. Am J Physiol Cell Physiol 284: C897-C909, 2003. doi:10.1152/ajpcell.00238.2002.
[60′] Bakhtiari S, Manshadi M K, Candas M, Beskok A. Changes in electrical capacitance of cell membrane reflect drug partitioning-induced alterations in lipid bilayer. Micromachines (Basel) 14: 316, 2023. doi:10.3390/mi14020316.
[61′] GUnzel D, Zakrzewski S S, Schmid T, Pangalos M, Wiedenhoeft J, Blasse C, Ozboda C, Krug S M. From TER to trans- and paracellular resistance: lessons from impedance spectroscopy. Ann N Y Acad Sci 1257: 142-151, 2012. doi:10.1111/j.1749-6632.2012.06540.x.
[62′] Weber W M, Cuppens H, Cassiman J J, Clauss W, Van Driessche W. Capacitance measurements reveal different pathways for the activation of CFTR. Pflugers Arch 438: 561-569, 1999. doi:10.1007/s004249900086.
[63′] Takahashi A, Watkins S C, Howard M, Frizzell R A. CFTR-dependent membrane insertion is linked to stimulation of the CFTR chloride conductance. Am J Physiol Cell Physiol 271: C1887-C1894, 1996. doi:10.1152/ajpcell.1996.271.6.C1887.
[64′] Suresh R, Swaminathan S, Rengaswamy R. Rapid impedance spectroscopy using dual phase shifted chirp signals for electrochemical applications. Int J Hydrogen Energy 45: 10536-10548, 2020. doi:10.1016/j.ijhydene.2019.10.031.
[65′] Quinn R H, Miller S S. Ion transport mechanisms in native human retinal pigment epithelium. Invest Ophthalmol Vis Sci 33: 3513-3527, 1992.
[66′] Maminishkis A, Chen S, Jalickee S, Banzon T, Shi G, Wang F E, Ehalt T, Hammer J A, Miller S S. Confluent monolayers of cultured human fetal retinal pigment epithelium exhibit morphology and physiology of native tissue. Invest Ophthalmol Vis Sci 47: 3612-3624, 2006. doi:10.1167/iovs.05-1622.
[67′] Korkka I, Skottman H, Nymark S. Heterogeneity of potassium channels in human embryonic stem cell-derived retinal pigment epithelium. Stem Cells Transl Med 11: 753-766, 2022. doi:10.1093/stcltm/szac029.
[68′] Okami T, Yamamoto A, Omori K, Takada T, Uyama M, Tashiro Y. Immunocytochemical localization of Na+,K(+)-ATPase in rat retinal pigment epithelial cells. J Histochem Cytochem 38: 1267-1275, 1990. doi:10.1177/38.9.2167328.
[69′] Erb L, Weisman G A. Coupling of P2Y receptors to G proteins and other signaling pathways. Wiley Interdiscip Rev Membr Transp Signal 1: 789-803, 2012. doi:10.1002/wmts.62.
[70′] S. Tokuda and A. S. L. Yu, “Regulation of Epithelial Cell Functions by the Osmolality and Hydrostatic Pressure Gradients: A Possible Role of the Tight Junction as a Sensor,” International Journal of Molecular Sciences, vol. 20, no. 14, p. 3513, 2019.
[71′] K. S. Spiegler, “Transport processes in ionic membranes,” Transactions of the Faraday Society, vol. 54, pp. 1408-1428, 1958.
[72′] S. A. Lewis and J. L. C. De Moura, “Apical Membrane Area of Rabbit Urinary Bladder Increases by Fusion of Intraceilular Vesicles: An Electrophysiological Study,” Tech. Rep., 1984, pp. 123-136.
[73′] S. G. Schultz, “Transport across epithelia: Some basic principles,” Kidney International, vol. 9, no. 2, pp. 65-75, 1976.
[74′] J. L. Kreindler, A. D. Jackson, P. A. Kemp, R. J. Bridges, and H. Danahay, “Inhibition of chloride secretion in human bronchial epithelial cells by cigarette smoke extract,” American Journal of Physiology—Lung Cellular and Molecular Physiology, vol. 288, no. 5 32-5, pp. 894-902, 2005.
[75′] Lewallen, Colby F., et al. “A biologically validated mathematical model for decoding epithelial apical, basolateral, and paracellular electrical properties.” American Journal of Physiology-Cell Physiology 325.6 (2023): C1470-C148

1-12. (canceled)

12. A system comprising:
an analysis system configured, via computer readable instructions to:
receive a response signal, or values thereof, of a measurement of a cell sample acquired from an impedance spectroscopy device or an associated data storage, wherein the cell sample had a first cell side associated with an apical membrane and a second cell side associated with a basolateral membrane, wherein the response signal or values thereof include (i) a first measurement across the first cell side or the second cell side between a first electrode and a second electrode; and
determine, via an impedance model of extracellular electrophysiology of a cell, based on the received response signal or values thereof, a first electrical characteristic value for the cell sample, a second electrical characteristic value, and a time-constant impedance-associated ratio,
wherein the first electrical characteristic value, the second electrical characteristic value, and the time-constant impedance-associated ratio are made accessible for use in a non-invasive characterization of the cell sample in response to a stimulus applied to the cell sample.

13. The system of claim 12, wherein the electrical characteristic values for the cell sample are determined by:
solving, via a mathematical solver, using the response signal or values thereof at a plurality of frequencies acquired via the impedance spectroscopy device, a resistance value and a capacitance value associated with the first cell side, a resistance value and a capacitance value associated with the second cell side, and a first resistance value and a second resistance value respective solutions in contact with the first and second cell side, wherein the response signal or values thereof include a voltage measure across the first electrode and the second electrode.

14. The system of claim 12, wherein the time-constant impedance-associated ratio can be used to provide an indication of membrane responses to the stimulus applied to the cell sample between the first cell side and the second cell side.

15. The system of claim 12, wherein the cell sample comprises a cell tissue.

16. The system of claim 12, wherein the first cell side is in contact with a first nutrient solution bath, and wherein the second cell side is in contact with a second nutrient solution bath, and wherein the first nutrient solution bath and the second nutrient solution bath are the same.

17. The system of claim 12, wherein the first cell side is in contact with a first nutrient solution bath, and wherein the second cell side is in contact with a second nutrient solution bath, wherein the first nutrient solution bath and the second nutrient solution bath are different.

18. The system of claim 12, wherein the impedance spectroscopy device comprises an impedance spectroscopy instrument.

19. The system of claim 12, wherein the impedance spectroscopy device is implemented as an external instrument driver device configured to couple to an impedance spectroscopy instrument.

20. The system of claim 12 further comprising:
a kit comprising:
a measurement chamber configured to house or culture the cell sample, wherein the cell sample, once placed in the measurement chamber; and
the two or more electrodes configured to be placed in the measurement chamber to measure the cell sample to be cultured or placed in the measurement chamber, the two or more electrodes having connectivity to an impedance spectroscopy device configured to interrogate the two or more electrodes once positioned in extracellular spaces, and optionally, intracellular spaces, to provide at least one response signals of the cell sample to current stimulation at different frequencies, wherein the at least one response signals of the cell sample can be employed in a model fitting operation to solve for electrical characteristic values for the cell sample, wherein one or more of the electrical characteristic values are made accessible for use in a non-invasive characterization of the cell sample in response to a stimulus applied to the cell sample.

21. The system of claim 20, wherein the two or more electrodes consist only of electrodes positioned in extracellular spaces of the measurement chamber, and wherein the at least one response signal provides for a first electrical characteristic value for the cell sample, a second electrical characteristic value, and a time-constant impedance-associated ratio to be used in the non-invasive characterization of the cell sample.

22. The system of claim 20, wherein the two or more electrodes consist of two electrodes positioned in extracellular spaces of the measurement chamber and a third electrode positioned in the intracellular space of the cell sample, and wherein the at least one response signals include (i) a first voltage measurement across the first cell side or the second cell side between a first electrode and a second electrode and (ii) a second voltage measurement across both the first and the second cell side between the first electrode and a third electrode, and wherein the at least one response signals provides for a resistance and capacitance characteristics of the first cell side, a resistance and capacitance characteristics of the second cell side, resistance characteristics of solutions in contact with the first and second cell side, and a resistance characteristic across the first and second cell side.

23-34. (canceled)

35. A method comprising:
culturing or placing a sample in a measurement chamber;
introducing a therapeutic agent, chemical agent, biological agent, compound, and/or environmental stimuli, to the cell sample; and
interrogating two or more electrodes positioned in extracellular spaces of the sample to provide a response signal of the cell sample,
wherein the response signal is made available to be assessed by an impedance model of extracellular electrophysiology of a cell for electrical characteristic values and a time-constant impedance-associated ratio for the cell sample, wherein the impedance model includes at least a resistance and capacitance characteristics of the first cell side, a resistance and capacitance characteristics of the second cell side, and
wherein one or more of the electrical characteristic values and the time-constant impedance-associated ratio are made accessible for use in a non-invasive characterization of the cell sample in response to a stimulus applied to the cell sample or non-invasive characterization of the stimulus, wherein the stimulus comprises at least one of a therapeutic agent, disease agent, or compound.

36. The method of claim 35, wherein the cell sample comprises a cell tissue.

37. The method of claim 35, wherein trends for the one or more of the electrical characteristic values are made accessible for use in the characterization of the cell sample or a stimulus applied to the cell sample.

38. The method of claim 35, wherein the therapeutic agent, chemical agent, biological agent, compound, and/or environmental stimuli are applied once, and a plurality of electrical characteristic values are measured for a pre-defined period of time or until equilibrium is observed in the measurement of the cell sample.

39. The method of claim 30, wherein the therapeutic agent, biological agent, compound, and/or environmental stimuli are applied in an on-going basis, and a plurality of electrical characteristic values are measured for a pre-defined period of time or until equilibrium is observed in the measurement of the cell sample.

40. The method of claim 35 further comprising:
monitoring, via a control unit, the one or more of the electrical characteristic values; and
triggering an introduction of a therapeutic agent, a biological agent, a compound, and/or environmental stimuli based on the monitored one or more of the electrical characteristic values.

41. The method of claim 35 further comprising:
evaluating electrical characteristics of a cell sample subjected to the therapeutic agent, chemical agent, biological agent, compound, and/or environmental stimuli by:
receiving at least two response signals, or values thereof, of a measurement of a cell sample acquired from an impedance spectroscopy device or an associated data storage, wherein the cell sample had a first cell side associated with an apical membrane and a second cell side associated with a basolateral membrane, wherein the at least two response signals or values thereof include (i) a first measurement across the first cell side or the second cell side between a first electrode and a second electrode and (ii) a second measurement across both the first and the second cell side between the first electrode and a third electrode; and
determining, via an impedance model of intracellular electrophysiology of a cell, based on the received two response signals or values thereof, electrical characteristic values for the cell sample, wherein the impedance model includes at least a resistance and capacitance characteristics of the first cell side, a resistance and capacitance characteristics of the second cell side, resistance characteristics of solutions in contact with the first and second cell side, and a resistance characteristic across the first and second cell side,
wherein one or more of the electrical characteristic values are made accessible for use in a non-invasive characterization of the cell sample in response to a stimulus applied to the cell sample or non-invasive characterization of the stimulus.

42. A non-transitory computer-readable medium having instructions stored thereon, wherein execution of the instructions by a processor causes the processor to:
receive a response signal, or values thereof, of a measurement of a cell sample acquired from an impedance spectroscopy device or an associated data storage, wherein the cell sample had a first cell side associated with an apical membrane and a second cell side associated with a basolateral membrane, wherein the response signal or values thereof include (i) a first measurement across the first cell side or the second cell side between a first electrode and a second electrode; and
determine, via an impedance model of extracellular electrophysiology of a cell, based on the received response signal or values thereof, a first electrical characteristic value for the cell sample, a second electrical characteristic value, and a time-constant impedance-associated ratio, wherein the first electrical characteristic value, the second electrical characteristic value, and the time-constant impedance-associated ratio are made accessible for use in a non-invasive characterization of the cell sample in response to a stimulus applied to the cell sample.

43. The non-transitory computer-readable medium of claim 42, wherein the therapeutic agent, biological agent, compound, and/or environmental stimuli are applied in an on-going basis, and a plurality of electrical characteristic values are measured for a pre-defined period of time or until equilibrium is observed in the measurement of the cell sample.

44. The non-transitory computer-readable medium of claim 42 wherein execution of the instructions by a processor causes the processor to:
monitor, via a control unit, the one or more of the electrical characteristic values; and
trigger an introduction of a therapeutic agent, a biological agent, a compound, and/or environmental stimuli based on the monitored one or more of the electrical characteristic values.
